(19) United States
(12) Patent Application Publication
(10) Pub. No.: US 2006/0241296 A1
(43) Pub. Date: Oct. 26, 2006

(54) Vanilloid receptor ligands and their use in treatments

(76) Inventors: Elizabeth M. Doherty, Thousand Oaks, CA (US); Jodie Katon, Woodland Hills, CA (US); Mark H. Norman, Thousand Oaks, CA (US); Daniel M. Retz, Thousand Oaks, CA (US); Xianghong Wang, Moorpark, CA (US); Yunxin Y. Bo, Thousand Oaks, CA (US); Nuria Tamayo, Newbury Park, CA (US); Nobuko Nishimura, West Hills, CA (US); Hongyu Liao, Thousand Oaks, CA (US)

Correspondence Address: AMGEN INC., MAIL STOP 28-2-C, ONE AMGEN CENTER DRIVE, THOUSAND OAKS, CA, 91320-1799, US

(21) Appl. No.: 11/411,366

(22) Filed: Apr. 25, 2006

Related U.S. Application Data
(60) Provisional application No. 60/674,732, filed on Apr. 25, 2005.

(51) Int. Cl. A61K 31/506; C07D 403/02

(52) U.S. Cl. 544/295; 544/324; 544/331

(57) ABSTRACT

Compounds having the general structure 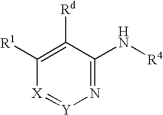 and compositions containing them, for the treatment of acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders.

This application claims the benefit of U.S. Provisional Application No. 60/674,732, filed Apr. 25, 2005, which is hereby incorporated by reference.

### BACKGROUND

The vanilloid receptor 1 (VR1) is the molecular target of capsaicin, the active ingredient in hot peppers. Julius et al. reported the molecular cloning of VR1 (Caterina et al., 1997). VR1 is a non-selective cation channel which is activated or sensitized by a series of different stimuli including capsaicin and resiniferatoxin (exogenous activators), heat & acid stimulation and products of lipid bilayer metabolism, anandamide (Premkumar et al., 2000, Szabo et al., 2000, Gauldie et al., 2001, Olah et al., 2001) and lipoxygenase metabolites (Hwang et al., 2000). VR1 is highly expressed in primary sensory neurons (Caterina et al., 1997) in rats, mice and humans (Onozawa et al., 2000, Mezey et al., 2000, Helliwell et al., 1998, Cortright et al., 2001). These sensory neurons innervate many visceral organs including the dermis, bones, bladder, gastrointestinal tract sand lungs; VR1 is also expressed in other neuronal and non-neuronal tissues including but not limited to, CNS nuclei, kidney, stomach and T-cells (Nozawa et al., 2001, Yiangou et al., 2001, Birder et al., 2001). Presumably expression in these various cells and organs may contribute to their basic properties such as cellular signaling and cell division.

Prior to the molecular cloning of VR1, experimentation with capsaicin indicated the presence of a capsaicin sensitive receptor, which could increase the activity of sensory neurons in humans, rats and mice (Holzer, 1991; Dray, 1992, Szallasi and Blumberg 1996, 1999). The results of acute activation by capsaicin in humans was pain at injection site and in other species increased behavioral sensitivity to sensory stimuli. (Szallasi and Blumberg, 1999). Capsaicin application to the skin in humans causes a painful reaction characterized not only by the perception of heat and pain at the site of administration but also by a wider are a of hyperalgesia and allodynia, two characteristic symptoms of the human condition of neuropathic pain (Holzer, 1991). Taken together, it seems likely that increased activity of VR1 plays a significant role in the establishment and maintenance of pain conditions. Topical or intradermal injection of capsaicin has also been shown to produce localized vasodilation and edema production (Szallasi and Blumberg 1999, Singh et al., 2001). This evidence indicates that capsaicin through it's activation of VR1 can regulate afferent and efferent function of sensory nerves. Sensory nerve involvement in diseases could therefore be modified by molecules which effect the function of the vanilloid receptor to increase or decrease the activity of sensory nerves.

VR1 gene knockout mice have been shown to have reduced sensory sensitivity to thermal and acid stimuli (Caterina et al., 2000)). This supports the concept that VR1 contributes not only to generation of pain responses (i.e. via thermal, acid or capsaicin stimuli) but also to the maintenance of basal activity of sensory nerves. This evidence agrees with studies demonstrating capsaicin sensitive nerve involvement in disease. Primary sensory nerves in humans and other species can be made inactive by continued capsaicin stimulation. This paradigm causes receptor activation induced desensitization of the primary sensory nerve—such reduction in sensory nerve activity in vivo makes subjects less sensitive to subsequent painful stimuli. In this regard both capsaicin and resinferatoxin (exogenous activators of VR1), produce desensitization and they have been used for many proof of concept studies in in vivo models of disease (Holzer, 1991, Dray 1992, Szallasi and Blumberg 1999).

### BIBLIOGRAPHY

Birder—L A. Kanai—A J. de-Groat—W C. Kiss—S. Nealen—M L. Burke—N E. Dineley—K E. Watkins—S. Reynolds—I J. Caterina—M J. (2001) Vanilloid receptor expression suggests a sensory role for urinary bladder epithelial cells. PNAS 98: 23: 13396-13401.
Caterina, M. J, Schumacher, M. A., Tominaga, M., Rosen, T. A., Levine, J. D., and Julius, D, (1997). The capsaicin receptor: a heat-activated ion channel in the pain pathway. Nature 389: 816-824.
Caterina—M J. Leffler—A. Malmberg—A B. Martin—W J. Trafton—J. Petersen-Zeitz K R. Koltzenburg—M. Basbaum—A I. Julius—D (2000) Impaired nociception and pain sensation in mice lacking the capsaicin receptor. Science—(Washington, D.C.). 288: 5464: 306-313.
Cortright—D N. Crandall—M. Sanchez—J F. Zou—T. Krause—J E. White—G (2001) The tissue distribution and functional characterization of human VR1. Biochemical and Biophysical Research Communications 281: 5: 1183-1189
Dray, A., (1992). Therapeutic potential of capsaicin-like molecules. Life Sciences 51: 1759-1765.
Gauldie—S D. McQueen—D S. Pertwee—R. Chessell—I P. (2001) Anandamide activates peripheral nociceptors in normal and arthritic rat knee joints. British Journal of Pharmacology 132: 3: 617-621.
Helliwell—R J A. McLatchie—L M. Clarke—M. Winter—J. Bevan—S. McIntyre—P (1998) Capsaicin sensitivity is associated with expression of the vanilloid (capsaicin) receptor (VR1) mRNA in adult rat sensory ganglia. Neuroscience Lett. 250: 3: 177-180.
Holzer, P. (1991) Capsaicin: Cellular targets, Mechanisms of Action and selectivity for thin sensory neurons. Pharmacological Reviews 43: 2: 143-201
Hwang—S W. Cho—H. Kwak—J. Lee—S Y. Kang—C J. Jung—J. Cho—S. Min—K H. Suh—Y G. Kim—D. Oh—U. (2000) Direct activation of capsaicin receptors by products of lipoxygenases: Endogenous capsaicin-like substances. PNAS 97: 11: 6155-6160.
Mezey—E. Toth—Z E. Cortright—D N. Arzubi—M K. Krause—J E. Elde—R. Guo—A. Blumberg—P M. Szallasi—A (2000) Distribution of mRNA for vanilloid receptor subtype 1 (VR1), and VR1-like immunoreactivity, in the central nervous system of the rat and human. PNAS 97: 7: 3655-3660.
Nozawa—Y. Nishihara—K. Yamamoto—A. Nakano—M. Ajioka—H. Matsuura—N. (2001) Distribution and characterization of vanilloid receptors in the rat stomach. Neuroscience Letters 309: 1: 33-36.
Olah—Z. Karai—L. Iadarola—M J. (2001) Anandamide activates vanilloid receptor 1 (VR1) at acidic pH in dorsal root ganglia neurons and cells ectopically expressing VR1. Journal of Biological Chemistry 276: 33, 31163-31170.
Onozawa—K. Nakamura—A. Tsutsumi—S. Yao—J. Ishikawa—R. Kohama—K. (2000) Tissue distribution of capsaicin receptor in the various organs of rats. Proc. Jpn. Acad. Ser. B, Phys.—Biol. Sci. 76: 5: 68-72.
Premkumar—L S. Ahem—G P. (2000) Induction of vanilloid receptor channel activity by protein kinase C. Nature (London) 408: 6815: 985-990.
Singh—L K. Pang—X. Alexacos—N. Letoumeau—R. Theoharides—T C. (1999) Acute immobilization stress triggers skin mast cell degranulation via corticotropin releasing hormone, neurotensin, and substance P: A link to neurogenic skin disorders. Brain Behav. Immun. 13: 3: 225-239.
Szallasi, A. Blumberg—P M (1996) Vanilloid receptors: New insights enhance potential as a therapeutic target. Pain 68: 195-208
Szallasi—A. Blumberg—P M. (1999) Vanilloid (capsaicin) receptors and mechanisms. Pharmacol. Rev. 51: 2: 159-211.
Szabo—T. Wang—J. Gonzalez—A. Kedei—N. Lile—J. Treanor—J. Blumberg—P M. (2000) Pharmacological characterization of the human vanilloid Receptor type-1 (hVR1). Society for Neuroscience Abstracts. 26:1-2: 634.18.
Tominaga, M., Caterina, M. J., Malmberg, A. B., Rosen, T. A., Gilbert, H., Skinner, K., Raumann, B. E., Basbaum, A. I., and Julius, D., (1998). The cloned capsaicin receptor integrates multiple pain-producing stimuli. Neuron 21: 531-543.
Yiangou—Y. Facer—P. Dyer—N H C. Chan—C L H. Knowles—C. Williams—N S. Anand—P. (2001) Vanilloid receptor 1 immunoreactivity in inflamed human bowel. Lancet (North American Edition) 357: 9265: 1338-1339.
Yiangou—Y. Facer—P. Ford—A. Brady—C. Wiseman—O. Fowler—C J. Anand—P. (2001) Capsaicin receptor VR1 and ATP-gated ion channel P2X3 in human urinary bladder. BJU International 87: 9: 774-779.
Wang—H. Bian—D. Zhu—D. Zajic—G. Loeloff—R. Lile—J. Wild—K. Treanor—J. Curran—E. (2000) Inflammation-induced upregulation of VR1 in rat spinal cord and DRG correlates with enhanced nociceptive processing. Society for Neuroscience Abstracts 26:1-2: 632.15.

### SUMMARY

The present invention comprises a new class of compounds useful in the treatment of diseases, such as vanilloid-receptor-mediated diseases and other maladies, such as inflammatory or neuropathic pain and diseases involving sensory nerve function such as asthma, rheumatoid arthritis, osteoarthritis, inflammatory bowel disorders, depression, anxiety, urinary incontinence, migraine and psoriasis. In particular, the compounds of the invention are useful for the treatment of acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders. Accordingly, the invention also comprises pharmaceutical compositions comprising the compounds, methods for the treatment of vanilloid-receptor-mediated diseases, such as inflammatory or neuropathic pain, asthma, rheumatoid arthritis, osteoarthritis, inflammatory bowel disorders, depression, anxiety, urinary incontinence, migraine and psoriasis diseases, using the compounds and compositions of the invention, and intermediates and processes useful for the preparation of the compounds of the invention.

The compounds of the invention are represented by the following general structure: 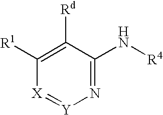
or a pharmaceutically acceptable salt thereof, wherein R¹, R⁴, R^{d}, X and Y are defined below.

The foregoing merely summarizes certain aspects of the invention and is not intended, nor should it be construed, as limiting the invention in any way. All patents, patent applications and other publications recited herein are hereby incorporated by reference in their entirety.

### DETAILED DESCRIPTION

One aspect of the current invention relates to compounds having the general structure: 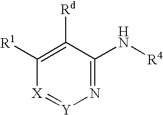
or any pharmaceutically-acceptable salt thereof, wherein:

X is N and Y is C(R³); or X is C(R²) and Y is N, n is independently, at each instance, 0, 1 or 2;

R¹ is 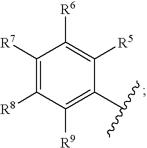
or R¹ is R^{b} substituted by 0, 1, 2 or 3 substituents selected from R^{e}, R^{h}, —OR^{f}, —OR^{h}, —OC₂₋₆alkylNR^{a}R^{f}, —OC₂₋₆alkylOR^{f}, —NR^{a}R^{f}, —NR^{a}R^{h}, —NR^{a}C₂₋₆alkylNR^{a}R^{f}, —NR^{a}C₂₋₆alkylOR^{f}, —CO₂R^{e}, —OC(═O)R^{e}, —C(═O)R^{e}, —C(═O)NR^{a}R^{f}, —C(═O)NR^{a}R^{h}, —NR^{a}C(═O)R^{e}, —NR^{a}C(═O)R^{h}, —NR^{a}C(═O)NR^{a}R^{f}, —NR^{a}CO₂R^{f}, —C₁₋₈alkylOR^{f}, —C₁₋₆alkylNR^{a}R^{f}, —S(═O)ₙR^{e}, —S(═O)₂NR^{a}R^{f}, —NR^{a}S(═O)₂R^{e}, —OS(═O)₂R^{e}, —OC(═O)NR^{a}R^{f}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} and —OC(═O)NR^{a}R^{h};

R² is selected from H, halo, cyano, nitro, Rⁱ, R^{k}, —OH, —ORⁱ, —OR^{k}, —S(O)ₙRⁱ, —S(O)ₙR^{k}, —N(R^{a})S(O)ₙRⁱ, —N(R^{a})S(O)ₙR^{k}, —S(O)ₙN(R^{a})Rⁱ, —S(O)ₙN(R^{a})R^{k}, —NH₂, —NR^{a}Rⁱ and —NR^{a}R^{k};

R³ is selected from H, halo, —NH₂, —NHC₁₋₃alkyl, —N(C₁₋₃alkyl)C₁₋₃alkyl, or C₁₋₃alkyl;

R⁴ is independently at each instance 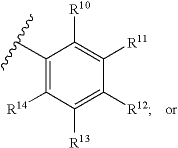
R⁴ is independently at each instance naphthyl substituted by OH, NH₂ or NHC₁₋₆alkyl, and additionally substituted by 0, 1, 2 or 3 substituents independently selected from R^{k}, C₁₋₄haloalkyl, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a}; and wherein the naphthyl is additionally substituted by 0 or 1 groups independently selected from Rⁱ and additionally substituted by 0, 1, 2, 3, 4 or 5 substituents independently selected from Br, Cl, F and I;

R⁵ is independently, at each instance, H, R^{k}, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a};

R⁶ is independently, at each instance, H, R^{k}, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a};

R⁷ is independently, at each instance, H, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}, —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h};

R⁸ is independently, at each instance, H, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R⁸, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h};

R⁹ is independently, at each instance, H, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h}; and

A)

R¹⁰ and R¹¹ together area saturated or partially unsaturated 3-, 4- or 5-carbon bridge, wherein the bridge is substituted by 0, 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; and

R¹² is independently, at each instance, selected from H, C₁₋₈alkyl, C₁₋₄haloalkyl, halo, cyano, nitro, —OH, —NH₂, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆OR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹² is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹² is C₁₋₄alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —OR, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(—O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ, and additionally substituted by 0, 1 or 2 halo groups; or

R¹⁰ is independently, at each instance, selected from H, halo, C₁₋₈alkyl, C₁₋₄haloalkyl, cyano, nitro, —C(═O)R^{k}, —C(═O)OR^{k}, —C(═O)NR^{a}R^{k}, —C(═NR^{a})NR^{a}R^{k}, —OH, —NH₂, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁰ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein there are no more than 2 N atoms, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁰ is C₁₋₄alkyl substituted by 0, 1, 2 or 3 groups selected from C₁₋₄haloalkyl, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; and

R¹¹ and R¹² together are a saturated or partially unsaturated 3-, 4- or 5-carbon bridge, wherein the bridge is substituted by 0, 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ;

R¹³ is independently, at each instance, selected from H, halo, cyano, nitro, C₁₋₄haloalkyl, —OH, —NH₂, C₁₋₈alkyl, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹³ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}. —NR^{a}C₂₋₆alkylNR^{a}Rⁱ, —NRⁱC₂₋₆alkylOR^{k} and —NR^{a}C₂₋₆alkylORⁱ; or R¹³ is C₁₋₄alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}R^{k}, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(R)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ;

R¹⁴ is independently, at each instance, selected from H, C₁₋₈alkyl, C₁₋₄haloalkyl, halo, cyano, nitro, —OH, —NH₂, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁴ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein there are no more than 2 N atoms, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁴ is C₁₋₄alkyl substituted by 0, 1, 2 or 3 groups selected from C₁₋₄haloalkyl, halo, cyano, nitro, —C(═O)R^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ;

R^{a} is independently, at each instance, H, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, —N(C₁₋₄alkyl)C₁₋₄alkyl;

R^{b} is a saturated or partially saturated 5-, 6- or 7-membered monocyclic or 6-, 7-, 8-, 9-, 10- or 11-membered bicyclic carbocyclic ring, or a saturated, partially saturated or unsaturated 5-, 6- or 7-membered monocyclic or 6-, 7-, 8-, 9-, 10- or 11-membered bicyclic ring containing 1, 2, 3 or 4 atoms selected from N, O and S, wherein the carbon atoms of any ring are substituted by 0, 1 or 2 oxo or thioxo groups, sulfur atoms of the ring are substituted by 0, 1 or 2 oxo groups, nitrogen atoms of the ring are substituted by 0 or 1 oxo groups;

R^{d} is independently in each instance hydrogen or —CH₃;

R^{e} is, independently, in each instance, C₁₋₉alkyl substituted by 0 or 1 groups independently selected from R^{h}; and wherein the C₁₋₉alkyl is additionally substituted by 0, 1, 2 or 3 substituents selected from halo, C₁₋₄haloalkyl, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a};

R^{f} is, independently, in each instance, H or R^{e};

R^{h} is, independently, in each instance, phenyl or a saturated, partially-saturated or unsaturated 5- or 6-membered monocyclic ring containing 1, 2 or 3 atoms selected from N, O and S, so long as the combination of O and S atoms is not greater than 2, wherein the ring is substituted by 0 or 1 oxo or thioxo groups, wherein the phenyl and monocycle are substituted by 0, 1 or 2 substituents selected from halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a};

Rⁱ is a saturated, partially saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, wherein the available carbon atoms of the ring are substituted by 0, 1 or 2 oxo or thioxo groups, wherein the ring is substituted by 0, 1, 2 or 3 substituents independently selected from C₁₋₉alkyl, halo, cyano, nitro, C₁₋₄haloalkyl, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, and C₁₋₉alkyl substituted by 1, 2 or 3 substituents selected from halo, cyano, nitro, C₁₋₄haloalkyl, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a}; and

R^{k} is, independently, in each instance, C₁₋₉alkyl or C₁₋₄alkyl(phenyl) wherein either is substituted by 0, 1, 2, 3 or 4 substituents selected from halo, C₁₋₄alkyl, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a}; and wherein the C₁₋₉alkyl is additionally substituted by 0 or 1 groups independently selected from Rⁱ and additionally substituted by 0, 1, 2, 3, 4 or 5 substituents independently selected from Br, Cl, F and I.

In one embodiment, in conjunction with any one of the above and below embodiments, R^{b} is a heterocycle selected from the group of thiophene, pyrrole, 1,3-oxazole, 1,3-thiazol-4-yl, 1,3,4-oxadiazole, 1,3,4-thiadiazole, 1,2,3-oxadiazole, 1,2,3-thiadiazole, 1H-1,2,3-triazole, isothiazole, 1,2,4-oxadiazole, 1,2,4-thiadiazole, 1,2,3,4-oxatriazole, 1,2,3,4-thiatriazole, 1H-1,2,3,4-tetraazole, 1,2,3,5-oxatriazole, 1,2,3,5-thiatriazole, furan, imidazol-2-yl, benzimidazole, 1,2,4-triazole, isoxazole, pyrazol-3-yl, pyrazol-4-yl, pyrazol-5-yl, thiolane, pyrrolidine, tetrahydrofuran, 4,5-dihydrothiophene, 2-pyrroline, 4,5-dihydrofuran, pyridazine, pyrimidine, pyrazine, 1,2,3-triazine, 1,2,4-triazine, 1,3,5-triazine, pyridine, 2H-3,4,5,6-tetrahydropyran, thiane, 1,2-diazaperhydroine, 1,3-diazaperhydroine, piperazine, 1,3-oxazaperhydroine, morpholine, 1,3-thiazaperhydroine, 1,4-thiazaperhydroine, piperidine, 2H-3,4-dihydropyran, 2,3-dihydro-4H-thiin, 1,4,5,6-tetrahydropyridine, 2H-5,6-dihydropyran, 2,3-dihydro-6H-thiin, 1,2,5,6-tetrahydropyridine, 3,4,5,6-tetrahydropyridine, 4H-pyran, 4H-thiin, 1,4-dihydropyridine, 1,4-dithiane, 1,4-dioxane, 1,4-oxathiane, 1,2-oxazolidine, 1,2-thiazolidine, pyrazolidine, 1,3-oxazolidine, 1,3-thiazolidine, imidazolidine, 1,2,4-oxadiazolidine, 1,3,4-oxadiazolidine, 1,2,4-thiadiazolidine, 1,3,4-thiadiazolidine, 1,2,4-triazolidine, 2-imidazolin-1-yl, 2-imidazolin-2-yl, 3-imidazoline, 2-pyrazoline, 4-imidazoline, 2,3-dihydroisothiazole, 4,5-dihydroisoxazole, 4,5-dihydroisothiazole, 2,5-dihydroisoxazole, 2,5-dihydroisothiazole, 2,3-dihydroisoxazole, 4,5-dihydrooxazole, 2,3-dihydrooxazole, 2,5-dihydrooxazole, 4,5-dihydrothiazole, 2,3-dihydrothiazole, 2,5-dihydrothiazole, 1,3,4-oxathiazolidine, 1,4,2-oxathiazolidine, 2,3-dihydro-1H-[1,2,3]triazole, 2,5-dihydro-1H-[1,2,3]triazole, 4,5-dihydro-1H-[1,2,3]triazol-1-yl, 4,5-dihydro-1H-[1,2,3]triazol-3-yl, 4,5-dihydro-1H-[1,2,3]triazol-5-yl, 2,3-dihydro-1H-[1,2,4]triazole, 4,5-dihydro-1H-[1,2,4]triazole, 2,3-dihydro-[1,2,4]oxadiazole, 2,5-dihydro-[1,2,4]oxadiazole, 4,5-dihydro-[1,2,4]thiadiazole, 2,3-dihydro-[1,2,4]thiadiazole, 2,5-dihydro-[1,2,4]thiadiazole, 4,5-dihydro-[1,2,4]thiadiazole, 2,5-dihydro-[1,2,4]oxadiazole, 2,3-dihydro-[1,2,4]oxadiazole, 4,5-dihydro-[1,2,4]oxadiazole, 2,5-dihydro-[1,2,4]thiadiazole, 2,3-dihydro-[1,2,4]thiadiazole, 4,5-dihydro-[1,2,4]thiadiazole, 2,3-dihydro-[1,3,4]oxadiazole, 2,3-dihydro-[1,3,4]thiadiazole, [1,4,2]oxathiazole, [1,3,4]oxathiazole, 1,3,5-triazaperhydroine, 1,2,4-triazaperhydroine, 1,4,2-dithiazaperhydroine, 1,4,2-dioxazaperhydroine, 1,3,5-oxadiazaperhydroine, 1,2,5-oxadiazaperhydroine, 1,3,4-thiadiazaperhydroine, 1,3,5-thiadiazaperhydroine, 1,2,5-thiadiazaperhydroine, 1,3,4-oxadiazaperhydroine, 1,4,3-oxathiazaperhydroine, 1,4,2-oxathiazaperhydroine, 1,4,5,6-tetrahydropyridazine, 1,2,3,4-tetrahydropyridazine, 1,2,3,6-tetrahydropyridazine, 1,2,5,6-tetrahydropyrimidine, 1,2,3,4-tetrahydropyrimidine, 1,4,5,6-tetrahydropyrimidine, 1,2,3,6-tetrahydropyrazine, 1,2,3,4-tetrahydropyrazine, 5,6-dihydro-4H-[1,2]oxazine, 5,6-dihydro-2H-[1,2]oxazine, 3,6-dihydro-2H-[1,2]oxazine, 3,4-dihydro-2H-[1,2]oxazine, 5,6-dihydro-4H-[1,2]thiazine, 5,6-dihydro-2H-[1,2] thiazine, 3,6-dihydro-2H-[1,2]thiazine, 3,4-dihydro-2H-[1,2]thiazine, 5,6-dihydro-2H-[1,3]oxazine, 5,6-dihydro-4H-[1,3]oxazine, 3,6-dihydro-2H-[1,3]oxazine, 3,4-dihydro-2H-[1,3]oxazine, 3,6-dihydro-2H-[1,4]oxazine, 3,4-dihydro-2H-[1,4]oxazine, 5,6-dihydro-2H-[1,3]thiazine, 5,6-dihydro-4H-[1,3]thiazine, 3,6-dihydro-2H-[1,3]thiazine, 3,4-dihydro-2H-[1,3]thiazine, 3,6-dihydro-2H-[1,4]thiazine, 3,4-dihydro-2H-[1,4]thiazine, 1,2,3,6-tetrahydro-[1,2,4]triazine, 1,2,3,4-tetrahydro-[1,2,4]triazine, 1,2,3,4-tetrahydro-[1,3,5]triazine, 2,3,4,5-tetrahydro-[1,2,4]triazine, 1,4,5,6-tetrahydro-[1,2,4]triazine, 5,6-dihydro-[1,4,2]dioxazine, 5,6-dihydro-[1,4,2]dithiazine, 2,3-dihydro-[1,4,2]dioxazine, 3,4-dihydro-2H-[1,3,4]oxadiazine, 3,6-dihydro-2H-[1,3,4]oxadiazine, 3,4-dihydro-2H-[1,3,5]oxadiazine, 3,6-dihydro-2H-[1,3,5]oxadiazine, 5,6-dihydro-2H-[1,2,5]oxadiazine, 5,6-dihydro-4H-[1,2,5]oxadiazine, 3,4-dihydro-2H-[1,3,4]thiadiazine, 3,6-dihydro-2H-[1,3,4]thiadiazine, 3,4-dihydro-2H-[1,3,5]thiadiazine, 3,6-dihydro-2H-[1,3,5]thiadiazine, 5,6-dihydro-2H-[1,2,5]thiadiazine, 5,6-dihydro-4H-[1,2,5]thiadiazine, 5,6-dihydro-2H-[1,2,3]oxadiazine, 3,6-dihydro-2H-[1,2,5]oxadiazine, 5,6-dihydro-4H-[1,3,4]oxadiazine, 3,4-dihydro-2H-[1,2,5]oxadiazine, 5,6-dihydro-2H-[1,2,3]thiadiazine, 3,6-dihydro-2H-[1,2,5]thiadiazine, 5,6-dihydro-4H-[1,3,4]thiadiazine, 3,4-dihydro-2H-[1,2,5]thiadiazine, 5,6-dihydro-[1,4,3]oxathiazine, 5,6-dihydro-[1,4,2]oxathiazine, 2,3-dihydro-[1,4,3]oxathiazine, 2,3-dihydro-[1,4,2]oxathiazine, 3,4-dihydropyridine, 1,2-dihydropyridine, 5,6-dihydropyridine, 2H-pyran, 2H-thiin, 3,6-dihydropyridine, 2,3-dihydropyridazine, 2,5-dihydropyridazine, 4,5-dihydropyridazine, 1,2-dihydropyridazine, 1,4-dihydropyrimidin-1-yl, 1,4-dihydropyrimidin-4-yl, 1,4-dihydropyrimidin-5-yl, 1,4-dihydropyrimidin-6-yl, 2,3-dihydropyrimidine, 2,5-dihydropyrimidine, 5,6-dihydropyrimidine, 3,6-dihydropyrimidine, 5,6-dihydropyrazine, 3,6-dihydropyrazine, 4,5-dihydropyrazine, 1,4-dihydropyrazine, 1,4-dithiin, 1,4-dioxin, 2H-1,2-oxazine, 6H-1,2-oxazine, 4H-1,2-oxazine, 2H-1,3-oxazine, 4H-1,3-oxazine, 6H-1,3-oxazine, 2H-1,4-oxazine, 4H-1,4-oxazine, 2H-1,3-thiazine, 2H-1,4-thiazine, 4H-1,2-thiazine, 6H-1,3-thiazine, 4H-1,4-thiazine, 2H-1,2-thiazine, 6H-1,2-thiazine, 1,4-oxathiin, 2H,5H-1,2,3-triazine, 1H,4H-1,2,3-triazine, 4,5-dihydro-1,2,3-triazine, 1H,6H-1,2,3-triazine, 1,2-dihydro-1,2,3-triazine, 2,3-dihydro-1,2,4-triazine, 3H,6H-1,2,4-triazine, 1H,6H-1,2,4-triazine, 3,4-dihydro-1,2,4-triazine, 1H,4H-1,2,4-triazine, 5,6-dihydro-1,2,4-triazine, 4,5-dihydro-1,2,4-triazine, 2H,5H-1,2,4-triazine, 1,2-dihydro-1,2,4-triazine, 1H,4H-1,3,5-triazine, 1,2-dihydro-1,3,5-triazine, 1,4,2-dithiazine, 1,4,2-dioxazine, 2H-1,3,4-oxadiazine, 2H-1,3,5-oxadiazine, 6H-1,2,5-oxadiazine, 4H-1,3,4-oxadiazine, 4H-1,3,5-oxadiazine, 4H-1,2,5-oxadiazine, 2H-1,3,5-thiadiazine, 6H-1,2,5-thiadiazine, 4H-1,3,4-thiadiazine, 4H-1,3,5-thiadiazine, 4H-1,2,5-thiadiazine, 2H-1,3,4-thiadiazine, 6H-1,3,4-thiadiazine, 6H-1,3,4-oxadiazine, and 1,4,2-oxathiazine, wherein the heterocycle is optionally vicinally fused with a saturated, partially-saturated or unsaturated 5-, 6- or 7-membered ring containing 0, 1 or 2 atoms independently selected from N, O and S.

In one embodiment, in conjunction with any one of the above and below embodiments, X is N and Y is C(R³).

In one embodiment, in conjunction with any one of the above and below embodiments, X is C(R²) and Y is N.

### Embodiment A

In another embodiment, in conjunction with any one of the above and below embodiments, R¹ is

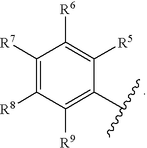

### Embodiment B

In another embodiment, in conjunction with any one of the above and below embodiments, R¹ is R^{b} is substituted by 1, 2 or 3 substituents selected from R^{e}, R^{h}, —OR^{f}, —OR^{h}, —OC₂₋₆alkylNR^{a}R^{f}, —OC₂₋₆alkylOR^{f}, —NR^{a}R^{f}, —NR^{a}R^{h}, —NR^{a}C₂₋₆alkylNR^{a}R^{f}, —NR^{a}C₂₋₆alkylOR^{f}, —CO₂R^{e}, —OC(═O)R^{e}, —C(═O)R^{e}, —C(═O)NR^{a}R^{f}, —C(═O)NR^{a}R^{h}, —NR^{a}C(═O)R^{e}, —NR^{a}C(═O)R^{h}, —NR^{a}C(═O)NR^{a}R^{f}, —NR^{a}CO₂R^{f}, —C₁₋₈alkylOR^{f}, —C₁₋₆alkylNR^{a}R^{f}, —S(═O)ₙR^{e}, —S(═O)₂NR^{a}R^{f}, —NR^{a}S(═O)₂R^{e}, —OS(═O)₂R^{e}, —OC(═O)NR^{a}R^{f}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} and —OC(═O)NR^{a}R^{h}.

### Embodiment C

In another embodiment, in conjunction with any one of the above and below embodiments, R¹ is

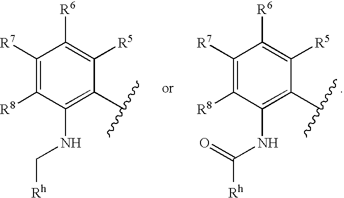

In another embodiment, in conjunction with any one of the above and below embodiments, R² is selected from H, halo, cyano, nitro, Rⁱ, R^{k}, —OH, —ORⁱ, —OR^{k}, —S(O)ₙRⁱ, —S(O)ₙR^{k}, —N(R^{a})S(O)ₙRⁱ, —N(R^{a})S(O)ₙR^{k}, —S(O)ₙN(R^{a})Rⁱ, —S(O)ₙN(R^{a})R^{k}, —NH₂, —NR^{a}Rⁱ and —NR^{a}R^{k}.

In another embodiment, in conjunction with any one of the above and below embodiments, R² is H.

In another embodiment, in conjunction with any one of the above and below embodiments, R² is selected from H and halo.

In another embodiment, in conjunction with any one of the above and below embodiments, R² is selected from Rⁱ, R^{k}, —OH, —ORⁱ, —OR^{k}, —S(O)ₙRⁱ, —S(O)ₙR^{k}, —N(R^{a})S(O)ₙRⁱ, —N(R^{a})S(O)ₙR^{k}, —S(O)ₙN(R^{a})Rⁱ, —S(O)ₙN(R^{a})R^{k}, —NH₂, —NR^{a}Rⁱ and —NR^{a}R^{k}.

In another embodiment, in conjunction with any one of the above and below embodiments, R² is a saturated, partially saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, wherein the available carbon atoms of the ring are substituted by 0, 1 or 2 oxo or thioxo groups, wherein the ring is substituted by 0, 1, 2 or 3 substituents independently selected from C₁₋₉alkyl, halo, cyano, nitro, C₁₋₄haloalkyl, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —OC₂₋₆alkylNR^{a}R^{a}, —OC₂₋₆alkylOR^{a}, —SR^{a}, —S(═O)R^{a}, —S(═O)₂NR^{a})R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, and C₁₋₉alkyl substituted by 1, 2 or 3 substituents selected from halo, cyano, nitro, C₁₋₄haloalkyl, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a}.

In another embodiment, in conjunction with any one of the above and below embodiments, R² is C₁₋₉alkyl or C₁₋₄alkyl(phenyl) wherein either is substituted by 0, 1, 2, 3 or 4 substituents selected from halo, C₁₋₄haloalkyl, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a}; and wherein the C₁₋₉alkyl is additionally substituted by 0 or 1 groups independently selected from Rⁱ and additionally substituted by 0, 1, 2, 3, 4 or 5 substituents independently selected from Br, Cl, F and I.

In another embodiment, in conjunction with any one of the above and below embodiments, R³ is selected from H, halo, —NH₂, —NHC₁₋₃alkyl, —N(C₁₋₃alkyl)C₁₋₃alkyl, or C₁₋₃alkyl.

In another embodiment, in conjunction with any one of the above and below embodiments, R³ is H.

In another embodiment, in conjunction with any one of the above and below embodiments, R³ is selected from H and halo.

In another embodiment, in conjunction with any one of the above and below embodiments, R³ is selected from halo, —NH₂, —NHC₁₋₃alkyl, —N(C₁₋₃alkyl)C₁₋₃alkyl, or C₁₋₃alkyl.

### Embodiment D

In another embodiment, in conjunction with any one of the above and below embodiments, R⁴ is independently at each instance

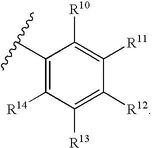

### Embodiment E

In another embodiment, in conjunction with any one of the above and below embodiments, R⁴ is independently at each instance

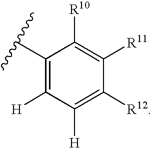

### Embodiment F

In another embodiment, in conjunction with any one of the above and below embodiments, R⁴ is independently at each instance

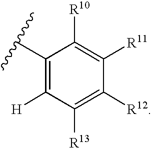

### Embodiment G

In another embodiment, in conjunction with any one of the above and below embodiments, R⁴ is independently at each instance

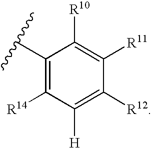

In another embodiment, in conjunction with any one of the above and below embodiments, R⁴ is naphthyl substituted by OH, NH₂ or NHC₁₋₆alkyl, and additionally substituted by 0, 1, 2 or 3 substituents independently selected from R^{k}, C₁₋₄haloalkyl, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a}; and wherein the naphthyl is additionally substituted by 0 or 1 groups independently selected from Rⁱ and additionally substituted by 0, 1, 2, 3, 4 or 5 substituents independently selected from Br, Cl, F and I.

In another embodiment, in conjunction with any one of the above and below embodiments, R⁵ is independently, at each instance, R^{k}, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a}.

In another embodiment, in conjunction with any one of the above and below embodiments, R⁵ is H.

In another embodiment, in conjunction with any one of the above and below embodiments, R⁵ is R^{k}.

In another embodiment, in conjunction with any one of the above and below embodiments, R⁵ is independently, at each instance, H, C₁₋₄haloalkyl, halo, —OR^{a}, —OC(═O)R^{a}, —NR^{a}R^{a}or —N(R^{a})C(═O)R^{a}.

In another embodiment, in conjunction with any one of the above and below embodiments, R⁶ is independently, at each instance, R^{k}, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a}.

In another embodiment, in conjunction with any one of the above and below embodiments, R⁶ is H.

In another embodiment, in conjunction with any one of the above and below embodiments, R⁶ is R^{k}.

In another embodiment, in conjunction with any one of the above and below embodiments, R⁶ is independently, at each instance, H, C₁₋₄haloalkyl, halo, —OR^{a}, —OC(═O)R^{a}, —NR^{a}R^{a}or —N(R^{a})C(═O)R^{a}.

In another embodiment, in conjunction with any one of the above and below embodiments, R⁷ is independently, at each instance, H, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C_{2 -6}alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h}.

### Embodiment H

In another embodiment, in conjunction with any one of the above and below embodiments, R⁷ is independently, at each instance, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h}.

In another embodiment, in conjunction with any one of the above and below embodiments, R⁷ is H.

In another embodiment, in conjunction with any one of the above and below embodiments, R⁷ is C₁₋₅alkyl, C₁₋₄haloalkyl, halo.

### Embodiment I

In another embodiment, in conjunction with any one of the above and below embodiments, R⁷ is independently, at each instance, C₃₋₅alkyl or C₁₋₂haloalkyl.

In another embodiment, in conjunction with any one of the above and below embodiments, R⁷ is C₃₋₅alkyl.

In another embodiment, in conjunction with any one of the above and below embodiments, R⁷ is —C(CH₃)₃.

In another embodiment, in conjunction with any one of the above and below embodiments, R⁷ is —CF₃.

In another embodiment, in conjunction with any one of the above and below embodiments, R⁸ is independently, at each instance, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h}.

In another embodiment, in conjunction with any one of the above and below embodiments, R⁸ is H.

In another embodiment, in conjunction with any one of the above and below embodiments, R⁸ is independently, at each instance, H, C₁₋₄haloalkyl, halo, —OR^{a}, —OC(═O)R^{a}, —NR^{a}R^{a}or —N(R^{a})C(═O)R^{a}.

### Embodiment J

In another embodiment, in conjunction with any one of the above and below embodiments, R⁹ is independently, at each instance, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}and —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h}.

### Embodiment K

In another embodiment, in conjunction with any one of the above and below embodiments, R⁹ is H.

### Embodiment L

In another embodiment, in conjunction with any one of the above and below embodiments, R⁹ is independently, at each instance, H, C₁₋₄haloalkyl, halo, —OR^{a}, —OC(═O)R^{a}, —NR^{a}R^{a}, —N(R^{a})C(═O)R^{a}.

In another embodiment, in conjunction with any one of the above and below embodiments, at least one of R⁵, R⁶, R⁷, R⁸ and R⁹ is tert-butyl or CF₃.

### Embodiment M

In another embodiment, in conjunction with any one of the above and below embodiments, R¹⁰ is independently, at each instance, selected from H, halo, C₁₋₈alkyl, C₁₋₄haloalkyl, cyano, nitro, —C(═O)R^{k}, —C(═O)OR^{k}, —C(═O)NR^{a}R^{k}, —C(═NR^{a})NR^{a}R^{k}, —OH, —NH₂, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁰ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein there are no more than 2 N atoms, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁰ is C₁₋₄alkyl substituted by 0, 1, 2 or 3 groups selected from C₁₋₄haloalkyl, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

In another embodiment, in conjunction with any one of the above and below embodiments, R¹⁰ is H.

In another embodiment, in conjunction with any one of the above and below embodiments, R¹⁰ is independently, at each instance, selected from halo, C₁₋₈alkyl, C₁₋₄haloalkyl, cyano, nitro, —C(═O)R^{k}, —C(═O)OR^{k}, —C(═O)NR^{a}R^{k}, —C(═NR^{a})NR^{a}R^{k}, —OH, —NH₂, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}Rⁱ, —NR^{a}C₂₋₆alkylNR^{a}Rⁱ, —NRⁱC₂₋₆alkylOR^{k} and —NR^{a}C₂₋₆alkylORⁱ; or R¹⁰ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein there are no more than 2 N atoms, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁰ is C₁₋₄alkyl substituted by 0, 1, 2 or 3 groups selected from C₁₋₄haloalkyl, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

### Embodiment N

In another embodiment, in conjunction with any one of the above and below embodiments, R¹⁰ and R¹¹ together are a saturated or partially unsaturated 3-, 4- or 5-carbon bridge, wherein the bridge is substituted by 0, 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

### Embodiment O

In another embodiment, in conjunction with any one of the above and below embodiments, R¹⁰ and R¹¹ together are a saturated or partially unsaturated 3-, 4-carbon bridge, wherein the bridge is substituted by 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

### Embodiment P

In another embodiment, in conjunction with any one of the above and below embodiments, R¹⁰ and R¹¹ together are a saturated or partially unsaturated 4-carbon bridge, wherein the bridge is substituted by 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

### Embodiment Q

In another embodiment, in conjunction with any one of the above and below embodiments, R¹² is independently, at each instance, selected from H, C₁₋₈alkyl, C₁₋₄haloalkyl, halo, cyano, nitro, —OH, —NH₂, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹² is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹² is C₁₋₄alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ, and additionally substituted by 0, 1 or 2 halo groups.

In another embodiment, in conjunction with any one of the above and below embodiments, R¹² is H.

In another embodiment, in conjunction with any one of the above and below embodiments, R¹² is independently, at each instance, selected from C₁₋₈alkyl, C₁₋₄haloalkyl, halo, cyano, nitro, —OH, —NH₂, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹² is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹² is C₁₋₄ alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ, and additionally substituted by 0, 1 or 2 halo groups.

In another embodiment, in conjunction with any one of the above and below embodiments, R¹² is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

### Embodiment R

In another embodiment, in conjunction with any one of the above and below embodiments, R¹¹ and R¹² together are a saturated or partially unsaturated 3-, 4- or 5-carbon bridge, wherein the bridge is substituted by 0, 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}R^{k}, —NR^{a}C₂₋₆alkylNR^{a}Rⁱ, —NRⁱC₂₋₆alkylOR^{k} and —NR^{a}C₂₋₆alkylORⁱ.

### Embodiment S

In another embodiment, in conjunction with any one of the above and below embodiments, R¹¹ and R¹² together are a saturated or partially unsaturated 4-carbon bridge, wherein the bridge is substituted by 0, 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

### Embodiment T

In another embodiment, in conjunction with any one of the above and below embodiments, R¹¹ and R¹² together are a saturated or partially unsaturated 4-carbon bridge, wherein the bridge is substituted by 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

In another embodiment, in conjunction with any one of the above and below embodiments, R¹³ is independently, at each instance, selected from H, halo, cyano, nitro, C₁₋₄haloalkyl, —OH, —NH₂, C₁₋₈alkyl, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹³ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹³ is C₁₋₄alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NRⁱRⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

In another embodiment, in conjunction with any one of the above and below embodiments, R¹³ is H.

In another embodiment, in conjunction with any one of the above and below embodiments, R¹³ is independently, at each instance, selected from halo, cyano, nitro, C₁₋₄haloalkyl, —OH, —NH₂, C₁₋₈alkyl, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹³ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR )NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹³ is C₁₋₄alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

In another embodiment, in conjunction with any one of the above and below embodiments, R¹³ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(—O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

In another embodiment, in conjunction with any one of the above and below embodiments, R¹³ is C₁₋₄alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

In another embodiment, in conjunction with any one of the above and below embodiments, R¹⁴ is independently, at each instance, selected from H, C₁₋₈alkyl, C₁₋₄haloalkyl, halo, cyano, nitro, —OH, —NH₂, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁴ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein there are no more than 2 N atoms, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁴ is C₁₋₄alkyl substituted by 0, 1, 2 or 3 groups selected from C₁₋₄haloalkyl, halo, cyano, nitro, —C(═O)R^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

In another embodiment, in conjunction with any one of the above and below embodiments, R¹⁴ is H.

In another embodiment, in conjunction with any one of the above and below embodiments, R¹⁴ is independently, at each instance, selected from C₁₋₈alkyl, C₁₋₄haloalkyl, halo, cyano, nitro, —OH, —NH₂, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, , —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁴ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein there are no more than 2 N atoms, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(—O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k}and —NR^{a}C₂₋₆alkylORⁱ; or R¹⁴ is C₁₋₄alkyl substituted by 0, 1, 2 or 3 groups selected from C₁₋₄haloalkyl, halo, cyano, nitro, —C(═O)R^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylOR^{k}SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ.

In another embodiment, in conjunction with any one of the above and below embodiments, at least one of R¹⁰, R¹¹, R¹², R¹³ and R¹⁴ is other than H.

In another embodiment, in conjunction with any one of the above and embodiments, at least two of R¹⁰, R¹¹, R¹², R¹³ and R¹⁴ is other than H.

As stated above, the above embodiments may be used in conjuction with embodiments listed. The following table is a non-exclusive, non-limiting list of some of the combinations of embodiments. Although the following embodiment sets are meant to be used with any of the above embodiments, they considered wherein R⁵, R⁶, R⁸, R¹³ and R¹⁴ are all H.

Where X is N and Y is CH:

| Emb. # | R¹ | R⁴ | R⁷ | R⁹ | R¹⁰ | R¹¹ | R¹² |
|---|---|---|---|---|---|---|---|
| 1001 | C | E | — | — | N | N | Q |
| 1002 | C | E | — | — | O | O | Q |
| 1003 | C | E | — | — | P | P | Q |
| 1004 | C | E | — | — | M | R | R |
| 1005 | C | E | — | — | M | S | S |
| 1006 | C | E | — | — | M | T | T |
| 1007 | C | D | — | — | — | — | — |
| 1008 | C | F | — | — | — | — | — |
| 1009 | C | G | — | — | — | — | — |
| 1010 | A | E | H | J | N | N | Q |
| 1011 | A | E | H | J | O | O | Q |
| 1012 | A | E | H | J | P | P | Q |
| 1013 | A | E | H | J | M | R | R |
| 1014 | A | E | H | J | M | S | S |
| 1015 | A | E | H | J | M | T | T |
| 1016 | A | D | H | J | — | — | — |
| 1017 | A | F | H | J | — | — | — |
| 1018 | A | G | H | J | — | — | — |
| 1019 | A | E | H | K | N | N | Q |
| 1020 | A | E | H | K | O | O | Q |
| 1021 | A | E | H | K | P | P | Q |
| 1022 | A | E | H | K | M | R | R |
| 1023 | A | E | H | K | M | S | S |
| 1024 | A | E | H | K | M | T | T |
| 1025 | A | D | H | K | — | — | — |
| 1026 | A | F | H | K | — | — | — |
| 1027 | A | G | H | K | — | — | — |
| 1028 | A | E | H | L | N | N | Q |
| 1029 | A | E | H | L | O | O | Q |
| 1030 | A | E | H | L | P | P | Q |
| 1031 | A | E | H | L | M | R | R |
| 1032 | A | E | H | L | M | S | S |
| 1033 | A | E | H | L | M | T | T |
| 1034 | A | D | H | L | — | — | — |
| 1035 | A | F | H | L | — | — | — |
| 1036 | A | G | H | L | — | — | — |
| 1037 | A | E | I | J | N | N | Q |
| 1038 | A | E | I | J | O | O | Q |
| 1039 | A | E | I | J | P | P | Q |
| 1040 | A | E | I | J | M | R | R |
| 1041 | A | E | I | J | M | S | S |
| 1042 | A | E | I | J | M | T | T |
| 1043 | A | D | I | J | — | — | — |
| 1044 | A | F | I | J | — | — | — |
| 1045 | A | G | I | J | — | — | — |
| 1046 | A | E | I | K | N | N | Q |
| 1047 | A | E | I | K | O | O | Q |
| 1048 | A | E | I | K | P | P | Q |
| 1049 | A | E | I | K | M | R | R |
| 1050 | A | E | I | K | M | S | S |
| 1051 | A | E | I | K | M | T | T |
| 1052 | A | D | I | K | — | — | — |
| 1053 | A | F | I | K | — | — | — |
| 1054 | A | G | I | K | — | — | — |
| 1055 | A | E | I | L | N | N | Q |
| 1056 | A | E | I | L | O | O | Q |
| 1057 | A | E | I | L | P | P | Q |
| 1058 | A | E | I | L | M | R | R |
| 1059 | A | E | I | L | M | S | S |
| 1060 | A | E | I | L | M | T | T |
| 1061 | A | D | I | L | — | — | — |
| 1062 | A | F | I | L | — | — | — |
| 1063 | A | G | I | L | — | — | — |
| 1064 | B | E | H | J | N | N | Q |
| 1065 | B | E | H | J | O | O | Q |
| 1066 | B | E | H | J | P | P | Q |
| 1067 | B | E | H | J | M | R | R |
| 1068 | B | E | H | J | M | S | S |
| 1069 | B | E | H | J | M | T | T |
| 1070 | B | D | H | J | — | — | — |
| 1071 | B | F | H | J | — | — | — |
| 1072 | B | G | H | J | — | — | — |
| 1073 | B | E | H | K | N | N | Q |
| 1074 | B | E | H | K | O | O | Q |
| 1075 | B | E | H | K | P | P | Q |
| 1076 | B | E | H | K | M | R | R |
| 1077 | B | E | H | K | M | S | S |
| 1078 | B | E | H | K | M | T | T |
| 1079 | B | D | H | K | — | — | — |
| 1080 | B | F | H | K | — | — | — |
| 1081 | B | G | H | K | — | — | — |
| 1082 | B | E | H | L | N | N | Q |
| 1083 | B | E | H | L | O | O | Q |
| 1084 | B | E | H | L | P | P | Q |
| 1085 | B | E | H | L | M | R | R |
| 1086 | B | E | H | L | M | S | S |
| 1087 | B | E | H | L | M | T | T |
| 1088 | B | D | H | L | — | — | — |
| 1089 | B | F | H | L | — | — | — |
| 1090 | B | G | H | L | — | — | — |
| 1091 | B | E | I | J | N | N | Q |
| 1092 | B | E | I | J | O | O | Q |
| 1093 | B | E | I | J | P | P | Q |
| 1094 | B | E | I | J | M | R | R |
| 1095 | B | E | I | J | M | S | S |
| 1096 | B | E | I | J | M | T | T |
| 1097 | B | D | I | J | — | — | — |
| 1098 | B | F | I | J | — | — | — |
| 1099 | B | G | I | J | — | — | — |
| 1100 | B | E | I | K | N | N | Q |
| 1101 | B | E | I | K | O | O | Q |
| 1102 | B | E | I | K | P | P | Q |
| 1103 | B | E | I | K | M | R | R |
| 1104 | B | E | I | K | M | S | S |
| 1105 | B | E | I | K | M | T | T |
| 1106 | B | D | I | K | — | — | — |
| 1107 | B | F | I | K | — | — | — |
| 1108 | B | G | I | K | — | — | — |
| 1109 | B | E | I | L | N | N | Q |
| 1110 | B | E | I | L | O | O | Q |
| 1111 | B | E | I | L | P | P | Q |
| 1112 | B | E | I | L | M | R | R |
| 1113 | B | E | I | L | M | S | S |
| 1114 | B | E | I | L | M | T | T |
| 1115 | B | D | I | L | — | — | — |
| 1116 | B | F | I | L | — | — | — |
| 1117 | B | G | I | L | — | — | — |

| Emb. # | R¹ | R⁴ | R⁷ | R⁹ | R¹⁰ | R¹¹ | R¹² |
|---|---|---|---|---|---|---|---|
| 2001 | C | E | — | — | N | N | Q |
| 2002 | C | E | — | — | O | O | Q |
| 2003 | C | E | — | — | P | P | Q |
| 2004 | C | E | — | — | M | R | R |
| 2005 | C | E | — | — | M | S | S |
| 2006 | C | E | — | — | M | T | T |
| 2007 | C | D | — | — | — | — | — |
| 2008 | C | F | — | — | — | — | — |
| 2009 | C | G | — | — | — | — | — |
| 2010 | A | E | H | J | N | N | Q |
| 2011 | A | E | H | J | O | O | Q |
| 2012 | A | E | H | J | P | P | Q |
| 2013 | A | E | H | J | M | R | R |
| 2014 | A | E | H | J | M | S | S |
| 2015 | A | E | H | J | M | T | T |
| 2016 | A | D | H | J | — | — | — |
| 2017 | A | F | H | J | — | — | — |
| 2018 | A | G | H | J | — | — | — |
| 2019 | A | E | H | K | N | N | Q |
| 2020 | A | E | H | K | O | O | Q |
| 2021 | A | E | H | K | P | P | Q |
| 2022 | A | E | H | K | M | R | R |
| 2023 | A | E | H | K | M | S | S |
| 2024 | A | E | H | K | M | T | T |
| 2025 | A | D | H | K | — | — | — |
| 2026 | A | F | H | K | — | — | — |
| 2027 | A | G | H | K | — | — | — |
| 2028 | A | E | H | L | N | N | Q |
| 2029 | A | E | H | L | O | O | Q |
| 2030 | A | E | H | L | P | P | Q |
| 2031 | A | E | H | L | M | R | R |
| 2032 | A | E | H | L | M | S | S |
| 2033 | A | E | H | L | M | T | T |
| 2034 | A | D | H | L | — | — | — |
| 2035 | A | F | H | L | — | — | — |
| 2036 | A | G | H | L | — | — | — |
| 2037 | A | E | I | J | N | N | Q |
| 2038 | A | E | I | J | O | O | Q |
| 2039 | A | E | I | J | P | P | Q |
| 2040 | A | E | I | J | M | R | R |
| 2041 | A | E | I | J | M | S | S |
| 2042 | A | E | I | J | M | T | T |
| 2043 | A | D | I | J | — | — | — |
| 2044 | A | F | I | J | — | — | — |
| 2045 | A | G | I | J | — | — | — |
| 2046 | A | E | I | K | N | N | Q |
| 2047 | A | E | I | K | O | O | Q |
| 2048 | A | E | I | K | P | P | Q |
| 2049 | A | E | I | K | M | R | R |
| 2050 | A | E | I | K | M | S | S |
| 2051 | A | E | I | K | M | T | T |
| 2052 | A | D | I | K | — | — | — |
| 2053 | A | F | I | K | — | — | — |
| 2054 | A | G | I | K | — | — | — |
| 2055 | A | E | I | L | N | N | Q |
| 2056 | A | E | I | L | O | O | Q |
| 2057 | A | E | I | L | P | P | Q |
| 2058 | A | E | I | L | M | R | R |
| 2059 | A | E | I | L | M | S | S |
| 2060 | A | E | I | L | M | T | T |
| 2061 | A | D | I | L | — | — | — |
| 2062 | A | F | I | L | — | — | — |
| 2063 | A | G | I | L | — | — | — |
| 2064 | B | E | H | J | N | N | Q |
| 2065 | B | E | H | J | O | O | Q |
| 2066 | B | E | H | J | P | P | Q |
| 2067 | B | E | H | J | M | R | R |
| 2068 | B | E | H | J | M | S | S |
| 2069 | B | E | H | J | M | T | T |
| 2070 | B | D | H | J | — | — | — |
| 2071 | B | F | H | J | — | — | — |
| 2072 | B | G | H | J | — | — | — |
| 2073 | B | E | H | K | N | N | Q |
| 2074 | B | E | H | K | O | O | Q |
| 2075 | B | E | H | K | P | P | Q |
| 2076 | B | E | H | K | M | R | R |
| 2077 | B | E | H | K | M | S | S |
| 2078 | B | E | H | K | M | T | T |
| 2079 | B | D | H | K | — | — | — |
| 2080 | B | F | H | K | — | — | — |
| 2081 | B | G | H | K | — | — | — |
| 2082 | B | E | H | L | N | N | Q |
| 2083 | B | E | H | L | O | O | Q |
| 2084 | B | E | H | L | P | P | Q |
| 2085 | B | E | H | L | M | R | R |
| 2086 | B | E | H | L | M | S | S |
| 2087 | B | E | H | L | M | T | T |
| 2088 | B | D | H | L | — | — | — |
| 2089 | B | F | H | L | — | — | — |
| 2090 | B | G | H | L | — | — | — |
| 2091 | B | E | I | J | N | N | Q |
| 2092 | B | E | I | J | O | O | Q |
| 2093 | B | E | I | J | P | P | Q |
| 2094 | B | E | I | J | M | R | R |
| 2095 | B | E | I | J | M | S | S |
| 2096 | B | E | I | J | M | T | T |
| 2097 | B | D | I | J | — | — | — |
| 2098 | B | F | I | J | — | — | — |
| 2099 | B | G | I | J | — | — | — |
| 2100 | B | E | I | K | N | N | Q |
| 2101 | B | E | I | K | O | O | Q |
| 2102 | B | E | I | K | P | P | Q |
| 2103 | B | E | I | K | M | R | R |
| 2104 | B | E | I | K | M | S | S |
| 2105 | B | E | I | K | M | T | T |
| 2106 | B | D | I | K | — | — | — |
| 2107 | B | F | I | K | — | — | — |
| 2108 | B | G | I | K | — | — | — |
| 2109 | B | E | I | L | N | N | Q |
| 2110 | B | E | I | L | O | O | Q |
| 2111 | B | E | I | L | P | P | Q |
| 2112 | B | E | I | L | M | R | R |
| 2113 | B | H | I | L | M | S | S |
| 2114 | B | E | I | L | M | T | T |
| 2115 | B | D | I | L | — | — | — |
| 2116 | B | F | I | L | — | — | — |
| 2117 | B | G | I | L | — | — | — |

Another aspect of the invention relates to a method of treating acute, inflammatory and neuropathic pain, dental pain, general headache, migraine, cluster headache, mixed-vascular and non-vascular syndromes, tension headache, inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders, comprising the step of administering a compound according to any of the above embodiments.

Another aspect of the invention relates to a pharmaceutical composition comprising a compound according to any of the above embodiments and a pharmaceutically-acceptable diluent or carrier.

Another aspect of the invention relates to the use of a compound according to any of the above embodiments as a medicament.

Another aspect of the invention relates to the use of a compound according to any of the above embodiments in the manufacture of a medicament for the treatment of acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders.

Another aspect of the invention relates to a method of making a compound according to the above embodiments, comprising the step of:
reacting 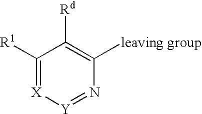
with R⁴NH to form

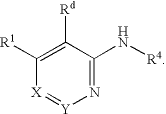

The compounds of this invention may have in general several asymmetric centers and are typically depicted in the form of racemic mixtures. This invention is intended to encompass racemic mixtures, partially racemic mixtures and separate enantiomers and diasteromers.

Unless otherwise specified, the following definitions apply to terms found in the specification and claims:
“C_{α-β}alkyl” means an alkyl group comprising a minimum of α and a maximum of β carbon atoms in a branched, cyclical or linear relationship or any combination of the three, wherein α and β represent integers. The alkyl groups described in this section may also contain one or two double or triple bonds. Examples of C₁₋₆alkyl include, but are not limited to the following: 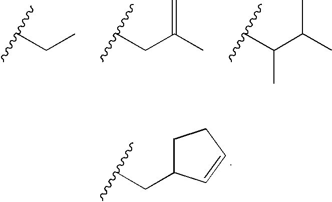
“Benzo group”, alone or in combination, means the divalent radical C₄H₄═, one representation of which is —CH═CH—CH═CH—, that when vicinally attached to another ring forms a benzene-like ring—for example tetrahydronaphthylene, indole and the like.
The terms “oxo” and “thioxo” represent the groups ═O (as in carbonyl) and ═S (as in thiocarbonyl), respectively.
“Halo” or “halogen” means a halogen atoms selected from F, Cl, Br and I.
“C_{v-w}haloalkyl” means an alkyl group, as described above, wherein any number—at least one—of the hydrogen atoms attached to the alkyl chain are replaced by F, Cl, Br or I.
“Heterocycle” means a ring comprising at least one carbon atom and at least one other atom selected from N, O and S. Examples of heterocycles that may be found in the claims include, but are not limited to, the following: 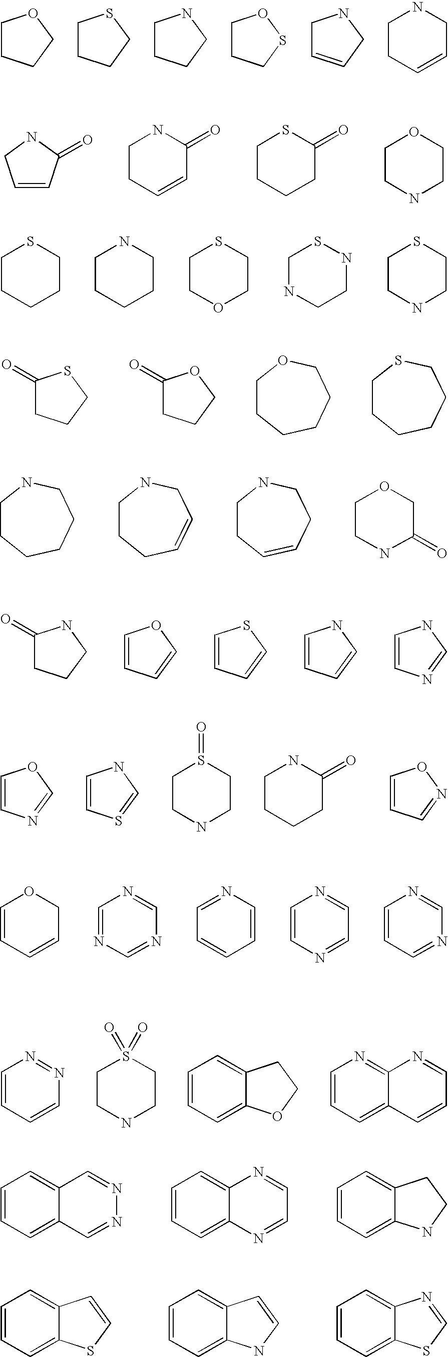 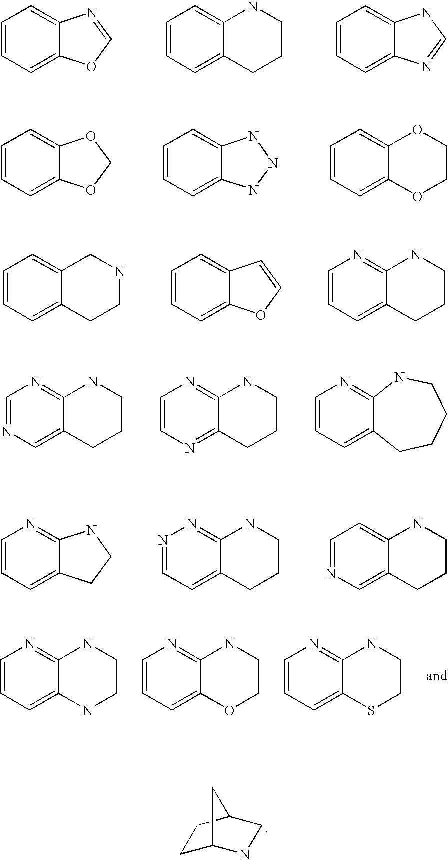
“Available nitrogen atoms” are those nitrogen atoms that are part of a heterocycle and are joined by two single bonds (e.g. piperidine), leaving an external bond available for substitution by, for example, H or CH₃.
“Pharmaceutically-acceptable salt” means a salt prepared by conventional means, and are well known by those skilled in the art. The “pharmacologically acceptable salts” include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).
“Saturated or unsaturated” includes substituents saturated with hydrogens, substituents completely unsaturated with hydrogens and substituents partially saturated with hydrogens.
“Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate.
“Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, trifluoroacetyl, trichloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

Silyl protecting groups are silicon atoms optionally substituted by one or more alkyl, aryl and aralkyl groups. Suitable silyl protecting groups include, but are not limited to, trimethylsilyl, triethylsilyl, triisopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino groups provide mono- or di-silylamino groups. Silylation of aminoalcohol compounds can lead to a N,N,O-trisilyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydrolysis and hydrogenolysis conditions well known to those skilled in the art.

It should be noted that compounds of the invention may contain groups that may exist in tautomeric forms, such as cyclic and acyclic amidine and guanidine groups, heteroatom substituted heteroaryl groups (Y′=O, S, NR), and the like, which are illustrated in the following examples: 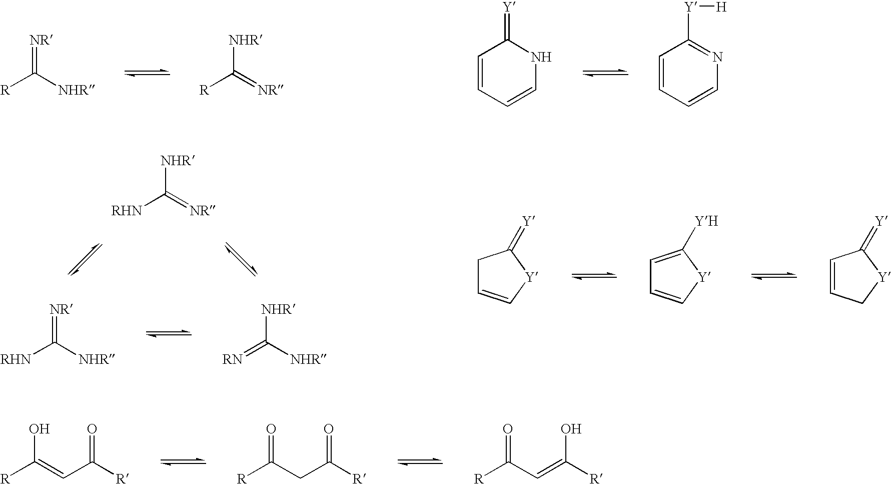
and though one form is named, described, displayed and/or claimed herein, all the tautomeric forms are intended to be inherently included in such name, description, display and/or claim.

Prodrugs of the compounds of this invention are also contemplated by this invention. A prodrug is an active or inactive compound that is modified chemically through in vivo physiological action, such as hydrolysis, metabolism and the like, into a compound of this invention following administration of the prodrug to a patient. The suitability and techniques involved in making and using prodrugs are well known by those skilled in the art. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives, which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

### EXPERIMENTAL

Unless otherwise noted, all materials were obtained from commercial suppliers and used without further purification. All parts are by weight and temperatures are in degrees centigrade unless otherwise indicated. Microwave assisted reactions were conducted with a Discover™ model microwave reactor (CEM, Matthews, N.C.) or a Smith Synthesizer™ (Personal Chemistry, Uppsala, Sweden). All compounds showed NMR spectra consistent with their assigned structures. Melting points were determined on a Buchi apparatus and are uncorrected. Mass spectral data was determined by electrospray ionization technique. All examples were purified to >90% purity as determined by high-performance liquid chromatography (HPLC).

The following abbreviations are used:
concd—concentrated
DMSO—dimethyl sulfoxide
DMF—N,N-dimethylformamide
THF—tetrahydrofuran
Et₂O—diethyl ether
EtOAc—ethyl acetate
MeOH—methyl alcohol
EtOH—ethyl alcohol
MeCN—acetonitrile
MeI—iodomethane
NMP—1-methyl-2-pyrrolidinone
DCM—dichloromethane
TBAF—tetrabutylammonium fluoride
TBS—t-butyldimethylsilyl
TFA—trifuoroacetic acid
satd—saturated
h—hours
min—minutes

### EXAMPLE 1

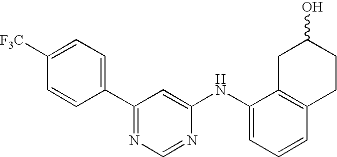

### 8-(6-(4-(Trifluoromethyl)phenyl)pyrimidin-4-ylamino)-1,2,3,4-tetrahydro-naphthalen-2-ol

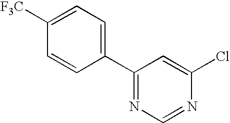 (a) 4-Chloro-6-[4-(trifluoromethyl)phenyl]pyrimidine.

To a 500-mL, round-bottomed flask was added 4,6-dichloropyrimidine (14 g, 95 mmol, Aldrich), 4-(trifluoromethyl)phenylboronic acid (6.0 g, 32 mmol, Aldrich), acetonitrile (95 mL) and 1 M aqueous solution of sodium carbonate (95 mL). The mixture was deoxygenated by sparging with N₂ for 15 min, and Pd(PPh₃)₄ (1.9 g, 1.6 mmol, Strem) was added. The resulting yellow mixture was heated at 80° C. with stirring for 15 h. After allowing to cool to 25° C., the mixture was evaporated under reduced pressure. The residue was diluted with 10% aqueous solution of NaHCO₃ and extracted with CH₂Cl₂. The combined extracts were dried over Na₂SO₄, filtered and evaporated under reduced pressure. The residue was purified by silica gel column chromatography (gradient: 1.5-10% EtOAc/hexane) to give the title compound as a white solid. MS (ESI, pos. ion) m/z: 259 [M+1]. 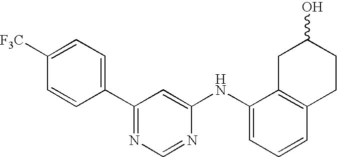
(b) 8-(6-(4-(Trifluoromethyl)phenyl)pyrimidin-4-ylamino)-1,2,3,4-tetrahydronaphthalen-2-ol.

A mixture of 4-chloro-6-[4-(trifluoromethyl)-phenyl]pyrimidine from step (a) above (0.35 g, 1.35 mmol) and 8-amino-1,2,3,4-tetrahydronaphthalen-2-ol (0.20 g, 1.2 mmol, prepared analogously to WO 2004/052846) in EtOH (2.5 mL) was heated in a microwave synthesizer at 160° C. for 20 min. The reaction mixture was diluted with EtOAc (100 mL), washed with satd NaHCO₃ (50 mL), water (50 mL) and satd NaCl (50 mL), dried over Na₂SO₄, filtered and evaporated under reduced pressure. Purification of the residue by silica gel column chromatography (gradient: 60-80% EtOAc in hexane) followed by re-crystallization of the product from EtOAc/hexane provided the title compound as off-white crystals. M.p.: 178-179° C. MS (ESI, pos. ion.) m/z: 386 [M+1].

### EXAMPLE 2 AND EXAMPLE 3

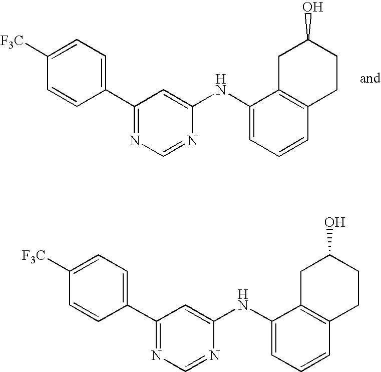

### (S)-8-(6-(4-(Trifluoromethyl)phenyl)pyrimidin-4-ylamino)-1,2,3,4-tetrahydronaphthalen-2-ol and (R)-8-(6-(4-(trifluoromethyl)phenyl)-pyrimidin-4-ylamino)-1,2,3,4-tetrahydronaphthalen-2-ol

Chiral separation (Chiralcel™ AD column; mobile phase: 25% EtOH in hexane; flow rate: 30 mL/min) of 8-(6-(4-(trifluoromethyl)phenyl)pyrimidin-4-ylamino)-1,2,3,4-tetrahydronaphthalen-2-ol (Example 1(b)) afforded the title compounds as brown amorphous solids. MS (ESI, pos. ion.) m/z: 386 [M+1]. The configuration (S) and (R) was assigned to each separated enantiomer at random.

### EXAMPLE 4

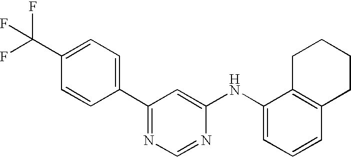

### N-(5,6,7,8-Tetrahydronaphthalen-1-yl)-6-(4-(trifluoromethyl)phenyl)-pyrimidin-4-amine

A mixture of 4-chloro-6-(4-(trifluoromethyl)phenyl) pyrimidine, Example 1(a), (0.26 g, 1.0 mmol) and 1,2,3,4-tetrahydro-1-naphthylamine (0.15 g, 1.0 mmol, Aldrich) in EtOH (1 mL) was heated in a microwave synthesizer at 120° C. for 15 min. The reaction mixture was evaporated under reduced pressure and the residue was dissolved in DCM. The DCM solution was washed with 10% sodium carbonate, dried over sodium sulfate and filtered. The filtrate was evaporated under reduced pressure and the residue purified by silica gel column chromatography (2:1:1 hexane/EtOAc/CHCl₃) to give the title compound as an off-white amorphous solid. MS (ESI, pos. ion) m/z: 370.

### EXAMPLE 5

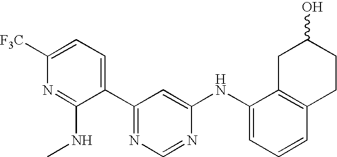

### 8-(6-(2-(Methylamino)-6-(trifluoromethyl)pyridin-3-yl)pyrimidin-4-ylamino)-1,2,3,4-tetrahydronaphthalen-2-ol

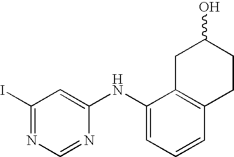 (a) 8-(6-Iodopyrimidin-4-ylamino)-1,2,3,4-tetrahydronaphthalen-2-ol.

A mixture of 2,6-diiodopyrimidine (2.0 g, 6.0 mmol, prepared according to Goodman, A. J.; Stanforth, S. P.; Tarbit, B. Desymmetrization of dichloro-azaheterocycles. Tetrahedron 1999, 55(52), 15067-15070) and 8-amino-1,2,3,4-tetrahydronaphthalen-2-ol (1.0 g, 6.1 mmol, prepared analogously to WO 2004/052846) in EtOH (20 mL) was stirred at reflux for 8 h. The reaction mixture was allowed to reach room temperature and the solvent was removed in vacuo. The dark-purple solid residue was partitioned between EtOAc (250 mL) and satd NaHCO₃ (100 mL). The organic layer was separated, washed with satd NaCl (50 mL), dried over Na₂SO₄, and filtered. The filtrate was evaporated in vacuo, and the solid residue was suspended in hot MeOH (100 mL). The suspension was diluted with hexane (500 mL) and the solid precipitate was filtered. The filter cake was separated and purified by silica gel column chromatography (gradient: 60-80% EtOAc/hexane) to give the title product as a purple solid. MS (ESI, pos. ion.) m/z: 368 [M+1]. 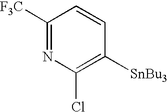
(b) 2-Chloro-3-(tributylstannyl)-6-(trifluoromethyl)pyridine.

To a solution of diisopropylamine (4.6 mL, 33 mmol, Aldrich) in anhydrous THF (40 mL) was added dropwise n-butyllithium (1.6 M in hexanes, 19 mL, 30 mmol, Aldrich) with stirring at −78° C. under N₂. Upon complete addition, the reaction mixture was stirred at −78° C. for 10 min, and at 0° C. for 10 min. The reaction mixture was cooled again to −78° C. and a solution of 2-chloro-6-trifluoromethylpyridine (5.0 g, 28 mmol, Oakwood) in anhydrous THF (15 mL) was added dropwise. The mixture was stirred at −78° C. for 2 h, tributyltin chloride (8.2 mL, 30 mmol, Aldrich) was added, and the stirring was continued for 2 h at −78° C. The reaction mixture was allowed to warm to −10° C. over 2 h and was quenched with a sat. aqueous solution of NH₄Cl (100 mL). The mixture was diluted with Et₂O (100 mL) and the organic layer was separated, washed with satd NaCl (75 mL), dried over Na₂SO₄, filtered, and concentrated in vacuo to provide an oil. The oil was purified by silica gel column chromatography (gradient: 0-5% EtOAc/hexane) to give the title product as a colorless oil. MS (ESI, pos. ion.) m/z: 471 [M+1]. 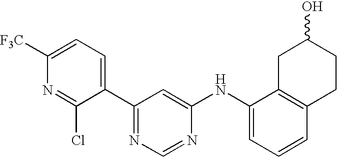
(c) 8-(6-(2-Chloro-6-(trifluoromethyl)pyridin-3-yl)pyrimidin-4-ylamino)-1,2,3,4-tetrahydronaphthalen-2-ol.

To a mixture of 2-chloro-3-(tributylstannyl)-6-(trifluoromethyl)pyridine from step (b) above (0.37 g, 0.79 mmol) and 8-(6-iodopyrimidin-4-ylamino)-1,2,3,4-tetrahydronaphthalen-2-ol from step (a) above (0.24 g, 0.65 mmol) in anhydrous DMF (5 mL) was added copper (I) iodide (0.050 g, 0.26 mmol, Aldrich, 98%) and tetrakis(triphenylphosphine)palladium(0) (0.038 g, 0.032 mmol, Strem) under Ar atmosphere. The mixture was heated at 60° C. with stirring for 1 h, left to reach 25° C., and diluted with EtOAc (50 mL) and water (50 mL). The layers were separated and the aqueous phase extracted with EtOAc (2×20 mL). The organic extracts were combined and washed with satd NaCl (30 mL), dried over Na₂SO₄, filtered and evaporated in vacuo. The residue was purified by silica gel column chromatography (gradient: 80-100% EtOAc/hexane) to give the title product as a yellow film. MS (ESI, pos. ion.) m/z: 421 [M+1]. 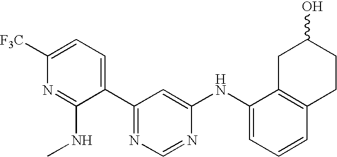
(d) 8-(6-(2-(Methylamino)-6-(trifluoromethyl)pyridin-3-yl)pyrimidin-4-ylamino)-1,2,3,4-tetrahydronaphthalen-2-ol.

To a 30 mL sealed tube was added 8-(6-(2-chloro-6-(trifluoromethyl)pyridin-3-yl)pyrimidin-4-ylamino)-1,2,3,4-tetrahydronaphthalen-2-ol from step (c) above (0.13 g, 0.30 mmol) and 30% methylamine in EtOH (10 mL, Aldrich). The tube was capped and heated at 80° C. in an oil bath with stirring for 18 h. The reaction mixture was allowed to reach room temperature and evaporated under reduced pressure. The residue was dissolved in chloroform, washed with 10% sodium carbonate, dried over sodium sulfate and filtered. The filtrate was evaporated under reduced pressure and the residue was purified by silica gel column chromatography, eluting with 2:1:1 hexane/ethyl acetate/chloroform and with 4:1 ethyl acetate/chloroform, to give the title compound as a pale yellow amorphous solid. MS (ESI, pos. ion) m/z: 416 [M+1].

### EXAMPLE 6

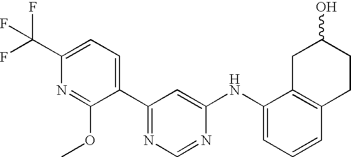

### 8-(6-(2-Methoxy-6-(trifluoromethyl)pyridin-3-yl)pyrimidin-4-ylamino)-1,2,3,4-tetrahydronaphthalen-2-ol

To a solution of 8-(6-(2-chloro-6-(trifluoromethyl)pyridin-3-yl)pyrimidin-4-ylamino)-1,2,3,4-tetrahydronaphthalen-2-ol, Example 5(c), (0.13 g, 0.30 mmol) in MeOH (5 mL) was added sodium (0.070 g, 3.0 mmol, Aldrich) and the mixture was stirred at 80° C. for 3 h. The reaction mixture was allowed to reach room temperature and was evaporated under reduced pressure. The residue was dissolved in dichloromethane and the solution was washed with satd sodium bicarbonate, dried over sodium sulfate, and filtered. The filtrate was evaporated under vacuo and the residue was purified by silica gel column chromatography (50% chloroform/ethyl acetate) to give the title compound as a white amorphous solid. MS (ESI, pos. ion) m/z: 417 [M+1].

### ADDITIONAL EXAMPLES

TABLE 1
The following examples were prepared from 8-(6-(2-chloro-6-
(trifluoromethyl)pyridin-3-yl)pyrimidin-4-ylamino)-1,2,3,4-tetrahydronaphthalen-
2-ol (Example 5(c)) and commercially available amines by following the
procedures described in Example 5(d), or with slight modifications thereof:

| | | MS* | Melting Point |
|---|---|---|---|
| No. | Structure | m/z | ° C. |
| 7 | 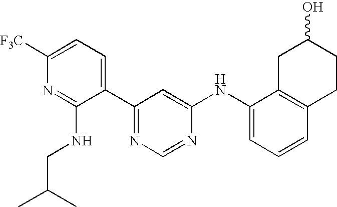 | 458 | Amorphous yellow solid |
| 8 | 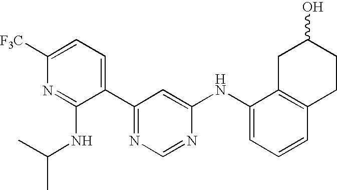 | 444 | Amorphous yellow solid |
| 9 | 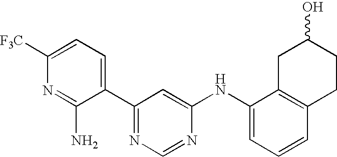 | 402 | Amorphous yellow solid |
| 10 | 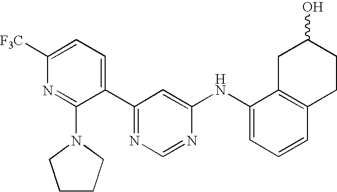 | 456 | Amorphous white solid |
| 11 | 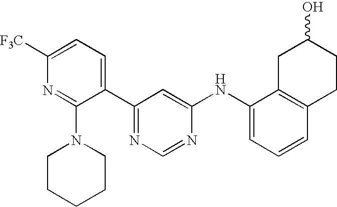 | 470 | Amorphous off-white solid |
| 12 | 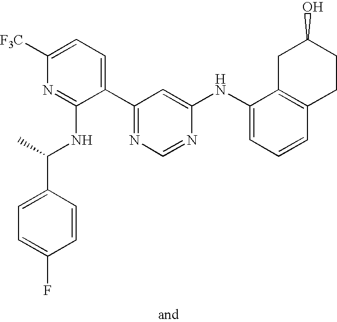 | 524 | Amorphous yellow solid |
| | 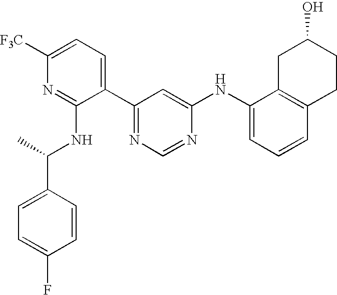 | | |
| 13 | 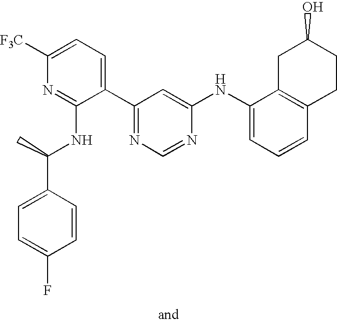 | 524 | Amorphous yellow solid |
| | 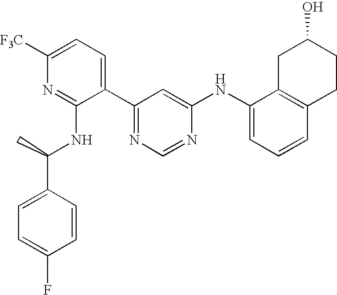 | | |
| 14 | 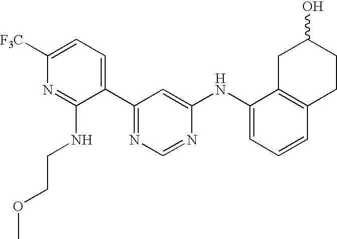 | 460 | Amorphous yellow solid |
| 15 | 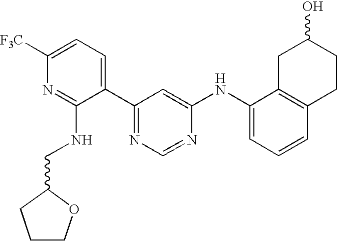 | 486 | Amorphous yellow solid |
| 16 | 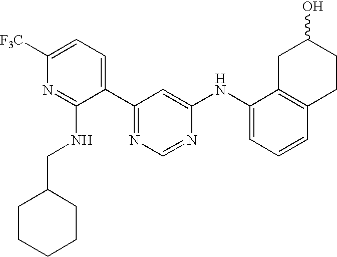 | 498 | Amorphous yellow solid |
| 17 | 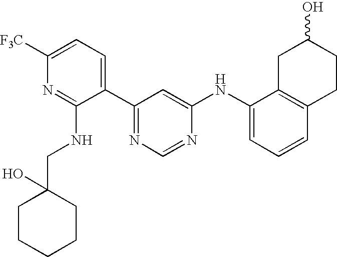 | 514 | Amorphous yellow solid |
| 18 | 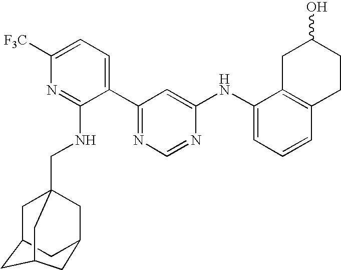 | 550 | Amorphous yellow solid |
| 19 | 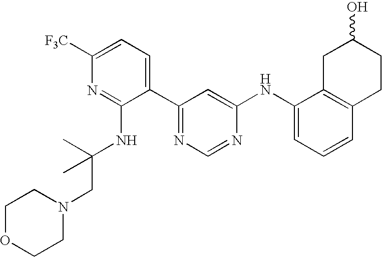 | 543 | Amorphous yellow solid |
| 20 | 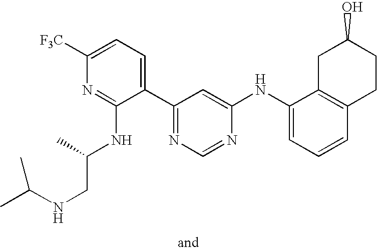 | 501 | Amorphous yellow solid |
| | 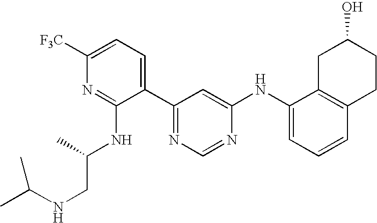 | | |
| 21 | 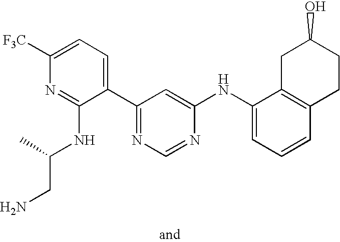 | 459 | Amorphous yellow solid |
| | 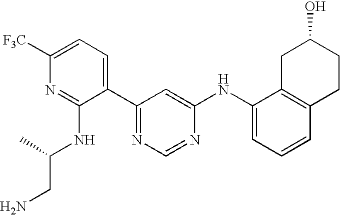 | | |
| 22 | 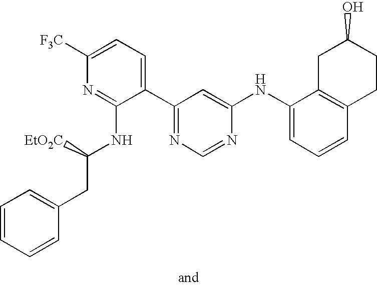 | 578 | Amorphous yellow solid |
| | 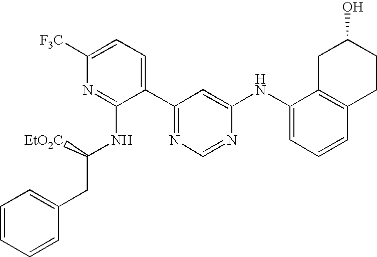 | | |
| 23 | 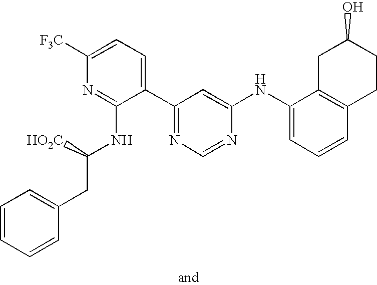 | 550 | Amorphous yellow solid |
| | 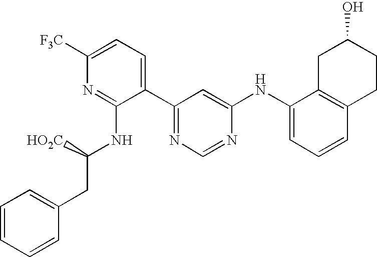 | | |

### EXAMPLE 24

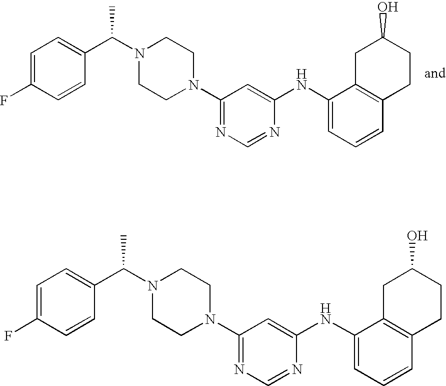

### (2S)-8-(6-(4-((1S)-1-(4-Fluorophenyl)ethyl)piperazin-1-yl)pyrimidin-4-ylamino)-1,2,3,4-tetrahydronaphthalen-2-ol and (2R)-8-(6-(4-((1S)-1-(4-Fluorophenyl)ethyl)piperazin-1-yl)pyrimidin-4-ylamino)-1,2,3,4-tetrahydro-naphthalen-2-ol

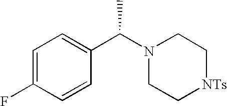 (a) (S)-1-(1-(4-Fluorophenyl)ethyl)-4-tosylpiperazine.

A mixture of N,N-bis(2-chloroethyl)-p-toluenesulfonamide (32 g, 110 mmol, Lancaster), (S)-4-(fluorophenyl)ethylamine (14 g, 98 mmol, Synquest) and N,N-diisopropyl-ethylamine (35 mL, Aldrich) was heated at 125° C. with stirring for 18 h. The reaction mixture was allowed to cool to 95° C., a 1:1 EtOH/water mixture (85 mL) was added dropwise, and the stirring was continued for 75 min. The reaction mixture was left to reach room temperature and the precipitated solids were filtered. The filter cake was washed with a 40:60 EtOH/water mixture (2×25 mL) and suspended in a 40:60 EtOH/water mixture (100 mL). The suspension was stirred for 75 min at room temperature and was filtered. The filter cake was washed with a 50:50 EtOH/water mixture (2×25 mL) and hexane (50 mL), and dried under vacuo at 55° C. for 18 h to give the title product as a brown solid. M.p.: 124-126° C. MS (ESI, pos. ion) m/z: 363 [M+1]. 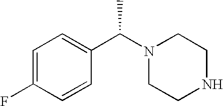
(b) (S)-1-[1-(4-Fluorophenyl)ethyl]-piperazine.

A mixture of (S)-1-[1-(4-fluoro-phenyl)-ethyl]-4-(toluene-4-sulfonyl)-piperazine from step (a) above (30 g, 83 mmol), 4-hydroxybenzoic acid (34 g, 240 mmol, Aldrich) and 33% solution of HBr in acetic acid (300 mL, Aldrich) was stirred at 25° C. for 46 h. The reaction mixture was diluted with water (350 mL) and was filtered. The filter cake was rinsed with water (75 mL). The acidic aqueous filtrates were combined and extracted with toluene (5×75 mL). The aqueous layer was separated, cooled in a water/ice bath, and treated with solid KOH (390 g) until pH>10. The mixture was diluted with water (400 mL) and extracted with toluene (3×100 mL) and ethyl acetate (2×75 mL). The combined organic extracts were dried over sodium sulfate, filtered and evaporated under reduced pressure. The residue was dried in vacuo to give the title product as a brown solid. M.p.: 62-64° C. MS (ESI, pos. ion) m/z: 209 [M+1]. 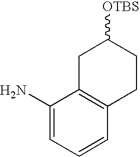
(c) 7-(tert-Butyldimethylsilyloxy)-5,6,7,8-tetrahydronaphthalen-1-amine.

A solution of 8-amino-1,2,3,4-tetrahydronaphthalen-2-ol (0.32 g, 2.0 mmol, prepared analogously to WO 2004/052846) in anhydrous DMF (5 mL) was stirred at 25° C. and treated with imidazole (0.27 g, 4.0 mmol, Aldrich) and tert-butyldimethylsilyl chloride (0.30 g, 2.1 mmol, Aldrich). The reaction mixture was stirred at 25° C. for 16 h, diluted with water (150 mL) and extracted with EtOAc (2×75 mL). The combined extracts were washed with water (50 mL), satd NaCl (50 mL), dried over Na₂SO₄, and filtered. The filtrate was evaporated under reduced pressure and the residue was dried in vacuo to afford the title product as dark-brown syrup. MS (ESI, pos. ion.) m/z: 146 (M-OTBS). 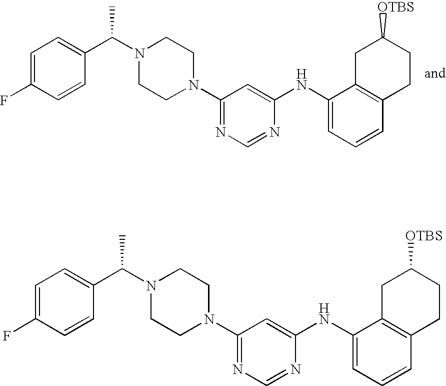
(d) N-((7S)-7-(tert-Butyldimethylsilyloxy)-5,6,7,8-tetrahydronaphthalen-1-yl)-6-(4-((1S)-1-(4-fluorophenyl)ethyl)piperazin-1-yl)pyrimidin-4-amine and N-((7R)-7-(tert-Butyldimethylsilyloxy)-5,6,7,8-tetrahydronaphthalen-1-yl)-6-(4-((1S)-1-(4-fluorophenyl)ethyl)piperazin-1-yl)pyrimidin-4-amine.

To a solution of 7-(tert-butyldimethylsilyloxy)-5,6,7,8-tetrahydronaphthalen-1-amine from step (c) above (0.45 g, 1.6 mmol) in anhydrous DMF (5 mL) was added K₂CO₃ (2 g, 14 mmol, Aldrich) and a solution of 4,6-difluoropyrimidine (0.17 g, 1.5 mmol, ABCR) in anhydrous DMF (1 mL) with stirring at room temperature. The mixture was heated at 60° C. with stirring for 68 h, left to reach room temperature, and diluted with EtOAc (100 mL). The reaction mixture was washed with 1 N HCl (2×75 mL), satd aqueous solution of NaHCO₃ (50 mL) and brine (50 mL), dried over MgSO₄, and filtered. The filtrate was evaporated under reduced pressure to afford a brown residue (0.17 g). To the residue was added (S)-1-(1-(4-fluorophenyl)ethyl)piperazine from step (b) above (0.10 g, 0.48 mmol), solid K₂CO₃ (0.20 g, 1.4 mmol, Aldrich) and anhydrous DMF (5 mL). The mixture was heated at 120° C. with stirring for 1 h and then allowed to cool to room temperature. The reaction mixture was diluted with EtOAc (75 mL), washed with satd aqueous solution of NaHCO₃ (50 mL) and brine (30 mL), dried over MgSO₄, and filtered. The filtrate was evaporated under reduced pressure and the residue purified by silica gel column chromatography (gradient: 50-100% EtOAc/hexane) to provide the title product as a mixture of diastereoisomers. 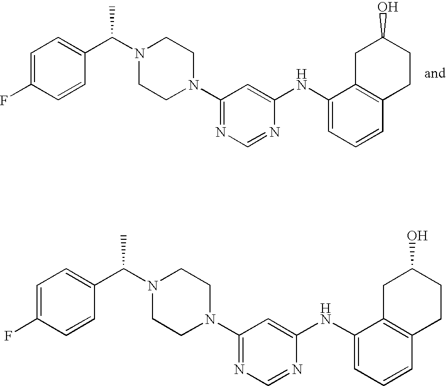
(e) (2S)-8-(6-(4-((1S)-1-(4-Fluorophenyl)ethyl)piperazin-1-yl)pyrimidin-4-ylamino)-1,2,3,4-tetrahydronaphthalen-2-ol and (2R)-8-(6-(4-((1S)-1-(4-Fluorophenyl)ethyl)piperazin-1-yl)pyrimidin-4-ylamino)-1,2,3,4-tetrahydro-naphthalen-2-ol.

N-((7S)-7-(tert-Butyldimethylsilyloxy)-5,6,7,8-tetrahydro-naphthalen-1-yl)-6-(4-((1S)-1-(4-fluorophenyl)ethyl)piperazin-1-yl)pyrimidin-4-amine and N-((7R)-7-(tert-Butyldimethylsilyloxy)-5,6,7,8-tetrahydronaphthalen-1-yl)-6-(4-((1S)-1-(4-fluorophenyl)ethyl)piperazin-1-yl)pyrimidin-4-amine from step (d) above (0.037 g, 0.066 mmol) was treated with 1 M TBAF in THF (1 mL, Aldrich) and stirred at 25° for 1 h. The reaction mixture was diluted with EtOAc (50 mL), washed with 10% Na₂CO₃ (20 mL), water (20 mL) and brine (10 mL), dried over Na₂SO₄, and filtered. The filtrate was evaporated under reduced pressure and the residue was purified by silica gel column chromatography (5% MeOH/EtOAc) to provide the title product as a mixture of diastereoisomers. MS (ESI, pos. ion.) m/z: 448 [M+1].

### EXAMPLE 25

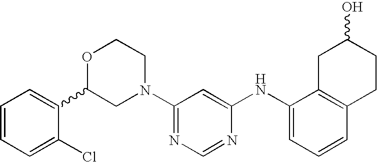

### (2R,S)-8-((5R,S)-6-(2-(2-Chlorophenyl)morpholino)pyrimidin-4-ylamino)-1,2,3,4-tetrahydronaphthalen-2-ol

A mixture of 2-(2-chlorophenyl)-morpholine oxalate (0.14 g, 0.49 mmol, Array Biopharma) and 8-(6-iodo-pyrimidin-4-ylamino)-1,2,3,4-tetrahydronaphthalen-2-ol, Example 5(a), (0.15 g, 0.41 mmol) in DMSO (3 mL) was heated in a microwave synthesizer at 140° C. for 1 h. The reaction mixture was quenched with satd aqueous solution of sodium bicarbonate (30 mL) and extracted with DCM (2×20 mL). The combined organic extracts were washed with water (20 mL), dried over sodium sulfate, filtered, and evaporated under reduced pressure. The residue was purified by silica gel column chromatography (gradient: 0-5% MeOH/DCM) to give the title compound as a mixture of diastereoisomers. MS (ESI, pos. ion) m/z: 437 [M+1].

### EXAMPLE 26

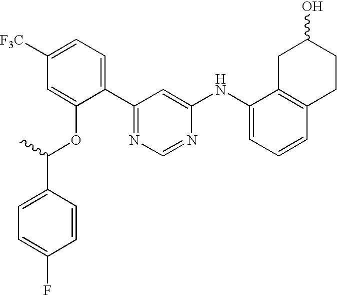

### (2R,S)-8-(6-(2-((1R,S)-1-(4-Fluorophenyl)ethoxy)-4-(trifluoromethyl)-phenyl)-pyrimidin-4-ylamino)-1,2,3,4-tetrahydronaphthalen-2-ol

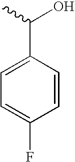 (a) 1-(4-Fluorophenyl)ethanol.

A solution of 4-fluoroacetophenone (10 g, 72 mmol, Aldrich) in EtOH (150 mL) was treated portionwise with NaBH₄ (7.0 g, 185 mmol, Aldrich) with stirring at 0° C. After the addition, the reaction mixture was stirred at room temperature for 2 h and quenched by the slow addition of acetone (20 mL). The mixture was evaporated under reduced pressure and the residue was dissolved in CH₂Cl₂ (250 mL). The solution was washed with water (2×200 mL) and brine (100 mL), dried over Na₂SO₄, and filtered. The filtrate was evaporated under reduced pressure and the residue dried in vacuo to afford the title product as a clear oil. 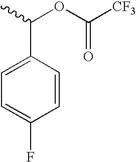
(b) 1-(4-Fluorophenyl)ethyl 2,2,2-trifluoroacetate.

To a solution of 1-(4-fluorophenyl)ethanol from step (a) above (5.0 g, 36 mmol) and triethylamine (6.0 mL, 43 mmol, Aldrich) in anhydrous CH₂Cl₂ (100 mL) was added dropwise trifluoroacetic anhydride (6.0 mL, 42 mmol, Aldrich) with stirring at 0° C. The reaction mixture was stirred at 0° C. for 30 min and washed consecutively with satd aqueous solution of NaHCO₃ (100 mL), 1 N HCl (100 mL) and brine (50 mL), dried over Na₂SO₄, and filtered. The filtrate was evaporated under reduced pressure and the residue dried in vacuo to afford the title product as a yellow oil. 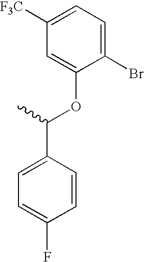
(c) 1-Bromo-2-(1-(4-fluorophenyl)ethoxy)-4-(trifluoromethyl)benzene.

[Analogous to the procedure described by Said, S. A.; Fiksdahl, A. Formation of chiral aryl ethers from enantiopure amine or alcohol substrates. Tetrahedron: Asymmetry 2001, 12(6), 893-896.] 2-Bromo-5-(trifluoromethyl)phenol (4.7 g, 19.5 mmol, prepared according to McBee, E. T.; Rapkin, E. Bromination of 3-(Trifluoromethyl)-phenol. J. Am. Chem. Soc. 1951, 73, 1325-1326) was added dropwise to a suspension of NaH (0.84 g, 21 mmol, 60% in mineral oil, Aldrich) in anhydrous DMF (40 mL) with stirring at 0° C. After the addition, the reaction mixture was stirred at 25° C. for 10 min, and treated with a solution of 1-(4-fluorophenyl)ethyl 2,2,2-trifluoroacetate from step (b) above (3.1 g, 13 mmol) in anhydrous DMF (2 mL). The reaction mixture was stirred at 80° C. for 14 h, and at 100° C. for 24 h. The reaction mixture was allowed to cool to 25° C., diluted with water (250 mL) and extracted with Et₂O (2×100 mL). The combined extracts were washed with 1 N NaOH (100 mL), water (100 mL) and brine (75 mL), dried over MgSO₄, and filtered. The filtrate was evaporated under reduced pressure and the residue was purified by silica gel column chromatography (gradient: 1-5% EtOAc/hexane) to provide the title product as a clear oil. 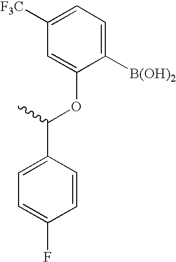
(d) 2-(1-(4-Fluorophenyl)ethoxy)-4-(trifluoromethyl)phenylboronic acid.

To a solution of 1-bromo-2-(1-(4-fluorophenyl)ethoxy)-4-(trifluoromethyl)benzene from step (c) above (1.9 g, 5.2 mmol) in anhydrous THF (40 mL) was added dropwise n-butyllithium (3.3 mL, 5.3 mmol, 1.6 M in hexanes, Aldrich) with stirring and cooling at −78° C. in a dry-ice/acetone bath under Ar atmosphere. Upon complete addition, the reaction mixture was stirred at −78° C. for 30 min, a solution of trimethyl borate (2.3 mL, 20 mmol, Aldrich) in anhydrous THF (5 mL) was added dropwise. The stirring was continued for 14 h, during which the reaction mixture was allowed to warm slowly to 25° C. The reaction mixture was cooled to 0° C., treated with 1 N HCl (50 mL), diluted with water (100 mL) and stirred vigorously at 25° C. for 1 h. The mixture was extracted with EtOAc (2×100 mL) and the combined extracts were washed successively with water, satd aqueous solution of NaHCO₃, 1 N HCl, water and brine, dried over MgSO₄, and filtered. The filtrate was evaporated under reduced pressure and the residue was purified by silica gel chromatography (gradient: 5-30% EtOAc/hexane) to provide the title product as a white amorphous solid. 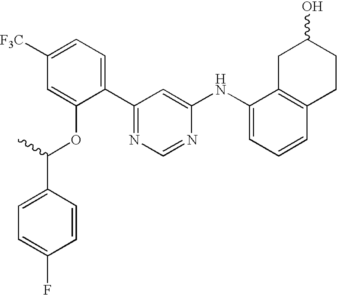
(e) (2R,S)-8-(6-(2-((1R,S)-(4-Fluorophenyl)ethoxy)-4-(trifluoromethyl)-phenyl)pyrimidin-4-ylamino)-1,2,3,4-tetrahydronaphthalen-2-ol.

A mixture of 2-(1-(4-fluorophenyl)ethoxy)-4-(trifluoromethyl)phenylboronic acid from step (d) above (0.20 g, 0.61 mmol), 8-(6-iodopyrimidin-4-ylamino)-1,2,3,4-tetrahydro-naphthalen-2-ol, Example 5(a), (0.25 g, 0.68 mmol), sodium carbonate (0.15 g, 1.2 mmol, J. T. Baker), DME (1.4 mL), EtOH (0.4 mL), and water (0.6 mL) was purged with Ar and treated with dichlorobis(triphenylphosphine)palladium(II) (0.043 g, 0.061 mmol, Aldrich). The reaction mixture was heated at 80° C. with stirring for 14 h under Ar atmosphere. The mixture was allowed to cool to 25° C. and was diluted with EtOAc. The resulting solution was washed with brine, dried over Na₂SO₄, and filtered. The filtrate was evaporated under reduced pressure and the residue was purified by silica gel chromatography (gradient: 65-85% EtOAc in hexane, followed by reverse-phase preparative HPLC [Phenomenex Synergi MAX-RP column, 0.1% TFA in CH₃CN/H₂O]. The fractions containing product were combined and concentrated under reduced pressure to remove CH₃CN. The resulting aqueous mixture was treated with satd NaHCO₃ and extracted with EtOAc. The organic extract was washed with brine, dried over Na₂SO₄, and filtered. The filtrate was evaporated under reduced pressure and the residue was dried in vacuo to afford the title product (0.10 g, 31%) as a mixture of diastereoisomers. MS (ESI, pos. ion.) m/z: 524 [M+1].

### EXAMPLE 27

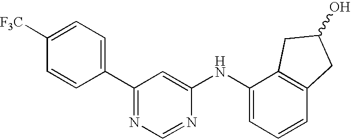

### 4-(6-(4-(Trifluoromethyl)phenyl)pyrimidin-4-ylamino)-2,3-dihydro-1H-inden-2-ol

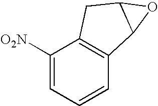 (a) 5-Nitro-6,6a-dihydro-1aH-1-oxa-cyclopropa[a]indene

To a solution of 7-nitro-1H-indene (5.3 g, 33 mmol, prepared according to Hahn, R. C.; Howard, P. H.; Lorenzo, G. A. Cyclopropane-arene interactions. II. Intramolecular charge transfer and geometry effects cyclopropyl nitroaromatic systems. J. Am. Chem. Soc. 1971, 93(22), 5816-5820) in CH₂Cl₂ (200 mL) was added portionwise 77% 3-chloroperoxybenzoic acid (12.5 g, 56 mmol, Aldrich) with stirring at 0° C. After the addition, the reaction mixture was stirred at 0° C. for 9 h and treated with an additional portion of 77% 3-chloroperoxybenzoic acid (6.8 g, 30 mmol, Aldrich). The reaction mixture was stirred at 0° C. for 3 h, and at 25° C. for 3 h. The mixture was washed with satd aqueous solution of NaHCO₃ (4×100 mL), 5% aqueous solution of Na₂SO₃ (2×100 mL), water (100 mL) and brine (50 mL), dried over Na₂SO₄, and filtered. The filtrate was evaporated under reduced pressure and the residue was dried in vacuo to afford the crude title product as a yellow oil, which was used in the next step without additional purification. 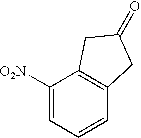
(b) 4-Nitro-1H-inden-2(3H)-one.

[Analogous to the procedure of Craig, J. C.; Torkelson, S. M.; Findell, P. R.; Weiner, R. I. Synthesis and dopaminergic activity of 2-substituted octahydrobenzo[f]quinolines. J. Med. Chem. 1989, 32(5), 961-968.] To a solution of 5-nitro-6,6a-dihydro-1aH-1-oxa-cyclopropa[a]indene from step (a) above (5.2 g, 29 mmol) in anhydrous toluene (100 mL) was added zinc iodide powder (3.7 g, 12 mmol, Aldrich) and the resulting mixture was heated at 85° C. with stirring for 4 h. The reaction mixture was allowed to cool to 25° C. and diluted with Et₂O (250 mL). The mixture was washed with water (100 mL) and brine (50 mL), dried over Na₂SO₄, and filtered. The filtrate was evaporated under reduced pressure and the residue purified by silica gel column chromatography (gradient: 5-30% EtOAc/hexane) to afford the title product as an orange waxy solid. 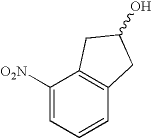
(c) 4-Nitro-2,3-dihydro-1H-inden-2-ol.

To a solution of 4-nitro-1H-inden-2(3H)-one from step (b) above (0.50 g, 2.8 mmol) in EtOH (10 mL) was added NaBH₄ (0.060 g, 1.6 mmol, Aldrich) with stirring at 25° C. The reaction mixture was stirred for 15 min at 25° C. and quenched with acetone (5 mL). The mixture was evaporated under reduced pressure and the residue was dissolved in EtOAc (100 mL). The solution was washed with satd. aqueous solution of NH₄Cl (50 mL), water (50 mL) and brine (50 mL), dried over Na₂SO₄, and filtered. The filtrate was evaporated under reduced pressure and the residue dried in vacuo to afford the title compound as a viscous brown oil. 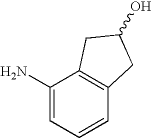
(d) 4-Amino-2,3-dihydro-1H-inden-2-ol.

A solution of 4-nitro-2,3-dihydro-1H-inden-2-ol from step (b) above (0.48 g, 2.7 mmol) in MeOH (10 mL) was purged with Ar and treated with 10% palladium on carbon (50 mg, Aldrich). The suspension was purged with H₂ and stirred under H₂ atmosphere for 2 h. After purging with Ar, the suspension was filtered through Celite® and the filtrate evaporated under reduced pressure. The residue was dried in vacuo to afford the title product (0.39 g, 98%) as a brown solid. MS (ESI, pos. ion.) m/z: 150 [M+1]. 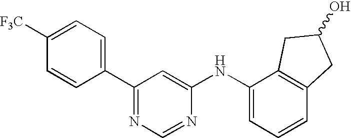
(e) 4-(6-(4-(Trifluoromethyl)phenyl)pyrimidin-4-ylamino)-2,3-dihydro-1H-inden-2-ol.

4-Amino-2,3-dihydro-1H-inden-2-ol from step (d) above (0.39 g, 2.6 mmol) was reacted with 4-chloro-6-[4-(trifluoromethyl)phenyl]pyrimidine, Example 1(a), (0.74 g, 2.9 mmol) under the conditions of Example 1(b) to give the title product as an amorphous white solid. MS (ESI, pos. ion.) m/z: 372 [M+1].

### EXAMPLE 28

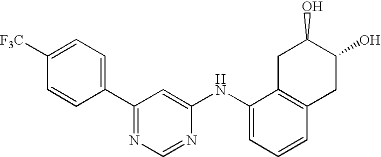

### (±)-5-(6-(4-(Trifluoromethyl)phenyl)pyrimidin-4-ylamino)-1,2,3,4-tetrahydronaphthalene-2,3-trans-diol

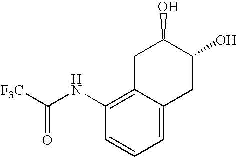 (a) (±)-N-(6,7-trans-Dihydroxy-5,6,7,8-tetrahydronaphthalen-1-yl)-2,2,2-trifluoroacetamide.

A solution of 2,2,2-trifluoro-N-(1a,2,7,7a-tetrahydro-naphtho[2,3-b]oxiren-3-yl)acetamide (0.50 g, 1.9 mmol, prepared according to the procedure described by: Rogers, G. A.; Parsons, S. M.; Anderson, D. C.; Nilsson, L. M.; Bahr, B. A.; Kornreich, W. D.; Kaufman, R.; Jacobs, R. S.; Kirtman, B. J. Med. Chem. 1989, 32, 1217-1230) in acetone (25 mL) was stirred at 25° C. and treated dropwise with 7% aqueous solution of HClO₄ (5 mL, prepared from 70% HClO₄, Aldrich). The resulting clear solution was stirred at room temperature for 15 h. The solution was evaporated under reduced pressure and the residue diluted with EtOAc (100 mL). The mixture was washed with satd aqueous solution of NaHCO₃ (50 mL) and brine (30 mL), dried over MgSO₄, and filtered. The filtrate was evaporated under reduced pressure and the residue dried in vacuo to give the title product as a white solid. MS (ESI, neg. ion) m/z: 274 (M−1). 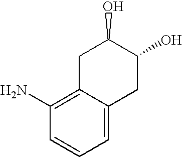
(b) (±)-5-Amino-1,2,3,4-tetrahydronaphthalene-2,3-trans-diol.

A mixture of (±)-N-(6,7-trans-dihydroxy-5,6,7,8-tetrahydronaphthalen-1-yl)-2,2,2-trifluoroacetamide from step (a) above (0.54 g, 2.0 mmol) and 2 M ammonia in MeOH (2.5 mL, 5.0 mmol, Aldrich) was heated in a microwave synthesizer at 100° C. for 40 min. The reaction mixture was evaporated under reduced pressure and the residue dried in vacuo to afford the crude title product as an off-white solid, which was used in the next step without additional purification. MS (ESI, pos. ion) m/z: 180 [M+1]. 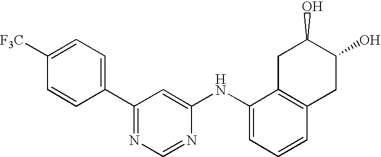
(c) (±)-5-(6-(4-(Trifluoromethyl)phenyl)pyrimidin-4-ylamino)-1,2,3,4-tetrahydronaphthalene-2,3-trans-diol.

(±)-5-Amino-1,2,3,4-tetrahydro-naphthalene-2,3-trans-diol from step (b) above (0.20 g, 1.1 mmol) was reacted with 4-chloro-6-(4-(trifluoromethyl)phenyl)pyrimidine, Example 1(a), (0.32 g, 1.2 mmol) under the conditions of Example 1(b) to give the title product as fine white crystals. M.p.: 286-289° C. MS (ESI, pos. ion) m/z: 402 [M+1].

### EXAMPLE 29

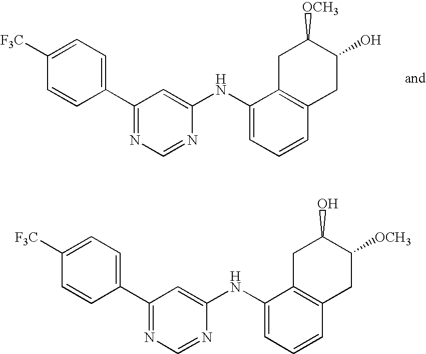

### 1:1 Mixture of (±)-trans-3-methoxy-5-(6-(4-(trifluoromethyl)phenyl)-pyrimidin-4-ylamino)-1,2,3,4-tetrahydronaphthalen-2-ol and (±)-trans-3-methoxy-8-(6-(4-(trifluoromethyl)phenyl)pyrimidin-4-ylamino)-1,2,3,4-tetrahydronaphthalen-2-ol

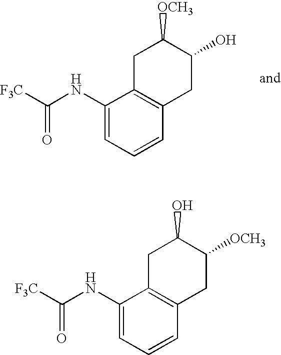 (a) 1:1 Mixture of 2,2,2-trifluoro-N-((±)-trans-6-hydroxy-7-methoxy-5,6,7,8-tetrahydronaphthalen-1-yl)acetamide and 2,2,2-trifluoro-N-((±)-trans-7-hydroxy-6-methoxy-5,6,7,8-tetrahydronaphthalen-1-yl)acetamide.

A mixture of 2,2,2-trifluoro-N-(1a,2,7,7a-tetrahydronaphtho[2,3-b]oxiren-3-yl)acetamide (0.50 g, 1.9 mmol, prepared according to the procedure described by: Rogers, G. A.; Parsons, S. M.; Anderson, D. C.; Nilsson, L. M.; Bahr, B. A.; Kornreich, W. D.; Kaufman, R.; Jacobs, R. S.; Kirtman, B. J. Med. Chem. 1989, 32, 1217-1230) and p-toluenesulfonic acid monohydrate (0.050 g, 0.26 mmol, Aldrich) in MeOH (25 mL) was stirred at 25° C. for 15 h. The reaction mixture was evaporated under reduced pressure and the residue was diluted with EtOAc (100 mL). The resulting solution was washed with satd aqueous solution of NaHCO₃ (50 mL) and brine (30 mL), dried over MgSO₄, and filtered. The filtrate was evaporated under reduced pressure and dried in vacuo to provide the crude 1:1 mixture of title compounds as an oily solid. The mixture was used in the next step without additional purification. MS (ESI, neg. ion) m/z: 288 (M−1). 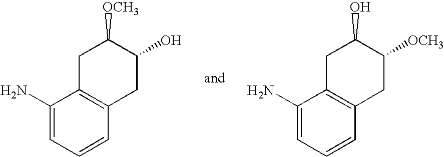
(b) 1:1 Mixture of (±)-5-amino-3-trans-methoxy-1,2,3,4-tetrahydro-naphthalen-2-ol and (±)-8-amino-3-trans-methoxy-1,2,3,4-tetrahydro-naphthalen-2-ol.

The 1:1 mixture of 2,2,2-trifluoro-N-((±)-trans-6-hydroxy-7-methoxy-5,6,7,8-tetrahydronaphthalen-1-yl)acetamide and 2,2,2-trifluoro-N-((±)-trans-7-hydroxy-6-methoxy-5,6,7,8-tetrahydronaphthalen-1-yl)acetamide from step (a) above (0.58 g, 2.0 mmol) was reacted with 2 M ammonia in MeOH (2.5 mL, 5.0 mmol, Aldrich) under the conditions of Example 28(b) to give the 1:1 mixture of title compounds as a colorless film. MS (ESI, pos. ion) m/z: 194 [M+1]. 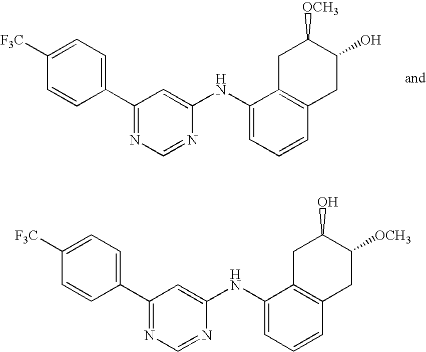
(c) 1:1 Mixture of (±)-trans-3-methoxy-5-(6-(4-(trifluoromethyl)phenyl)-pyrimidin-4-ylamino)-1,2,3,4-tetrahydronaphthalen-2-ol and (±)-trans-3-methoxy-8-(6-(4-(trifluoromethyl)phenyl)pyrimidin-4-ylamino)-1,2,3,4-tetrahydronaphthalen-2-ol.

A 1:1 mixture of (±)-5-amino-3-trans-methoxy-1,2,3,4-tetrahydronaphthalen-2-ol and (±)-8-amino-3-trans-methoxy-1,2,3,4-tetrahydronaphthalen-2-ol from step (b) above (0.30 g, 1.6 mmol) was reacted with 4-chloro-6-(4-(trifluoromethyl)phenyl)pyrimidine, Example 1(a), (0.40 g, 1.5 mmol) under the conditions of Example 1(b) to give the 1:1 mixture of the title compounds as a white amorphous solid. MS (ESI, pos. ion) m/z: 416 [M+1].

### EXAMPLE 30

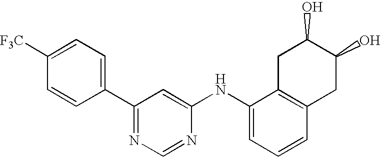

### (±)-5-(6-(4-(Trifluoromethyl)phenyl)pyrimidin-4-ylamino)-1,2,3,4-tetrahydronaphthalene-2,3-cis-diol

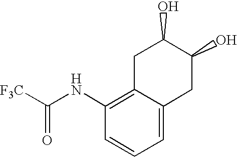 (a) N-((±)-6,7-cis-Dihydroxy-5,6,7,8-tetrahydronaphthalen-1-yl)-2,2,2-trifluoroacetamide.

A solution of N-(5,8-dihydronaphthalen-1-yl)-2,2,2-trifluoroacetamide (5.0 g, 21 mmol, prepared according to the procedure described by: Rogers, G. A.; Parsons, S. M.; Anderson, D. C.; Nilsson, L. M.; Bahr, B. A.; Kornreich, W. D.; Kaufman, R.; Jacobs, R. S.; Kirtman, B. J. Med. Chem. 1989, 32, 1217-1230) in acetone (150 mL), distilled water (20 mL) and 2-methylpropan-2-ol (13 mL) was stirred at 25° C. and treated with 4-methylmorpholine N-oxide (4.1 g, 35 mmol, Aldrich) and potassium osmate (VI) dihydrate (0.053 g, 0.14 mmol, Strem). The reaction mixture was stirred at 25° C. for 17 h then treated with NaHSO₃ (2 g) to decompose OSO₄. After 15 min stirring, the solvent was removed in vacuo to provide a dark solid. The solid was suspended in EtOAc (500 mL), treated with MgSO₄ (10 g), and filtered through Celite®. The filter cake was washed with acetone (500 mL). The combined filtrate was evaporated under reduced pressure and dried in vacuo for 17 h to afford a tan solid. The crude product was purified by silica gel column chromatography, eluting with a step gradient of 80% EtOAc in hexane followed by 100% EtOAc, to provide the title product as an off-white solid. MS (ESI, pos. ion) m/z: 276 [M+1]. 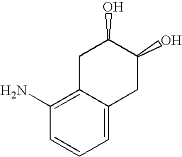
(b) (±)-5-Amino-1,2,3,4-tetrahydronaphthalene-2,3-cis-diol.

N-((±)-6,7-cis-Dihydroxy-5,6,7,8-tetrahydronaphthalen-1-yl)-2,2,2-trifluoroacetamide from step (a) above (0.53 g, 1.9 mmol) was reacted with 2.0 M ammonia in MeOH (5 mL, 10 mmol, Aldrich) under the conditions of Example 28(b) to give the title product as a pale-tan solid. MS (ESI, pos. ion) m/z: 180 [M+1]. 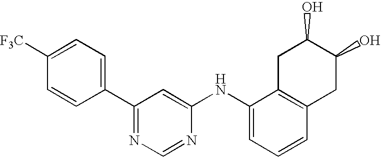
(c) (±)-5-(6-(4-(Trifluoromethyl)phenyl)pyrimidin-4-ylamino)-1,2,3,4-tetrahydronaphthalene-2,3-cis-diol.

(±)-5-Amino-1,2,3,4-tetrahydro-naphthalene-2,3-cis-diol from step (b) above (0.12 g, 0.67 mmol) was reacted with 4-chloro-6-(4-(trifluoromethyl)phenyl)pyrimidine, Example 1a, (0.19 g, 0.73 mmol) under the conditions of Example 1(b) to give the title product as a white crystalline solid. M.p.: 231-232° C. MS (ESI, pos. ion) m/z: 402 [M+1].

### EXAMPLE 31

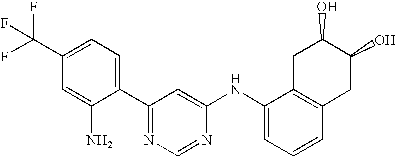

### (±)-5-(6-(2-Amino-4-(trifluoromethyl)phenyl)pyrimidin-4-ylamino)-1,2,3,4-tetrahydronaphthalene-2,3-cis-diol

Following the procedure described for Example 1b, (±)-5-amino-1,2,3,4-tetrahydronaphthalene-2,3-cis-diol, Example 30b, (0.67 g, 3.7 mmol) and tert-butyl 2-(6-chloropyrimidin-4-yl)-5-(trifluoromethyl)phenylcarbamate (1.4 g, 3.7 mmol, prepared according to WO 2004/014871) provided the title product as an off-white crystalline solid. M.p.: 233-234° C. MS (ESI, pos. ion) m/z: 417 [M+1].

### EXAMPLE 32

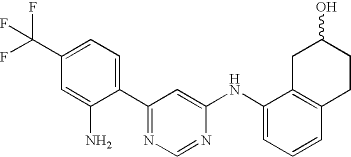

### 8-(6-(2-Amino-4-(trifluoromethyl)phenyl)pyrimidin-4-ylamino)-1,2,3,4-tetrahydronaphthalen-2-ol

tert-Butyl 2-(6-chloropyrimidin-4-yl)-5-(trifluoromethyl)phenyl-carbamate (3.7 g, 10 mmol, prepared according to WO 2004/014871) was reacted with 8-amino-1,2,3,4-tetrahydronaphthalen-2-ol (1.6 g, 10 mmol, prepared analogously to WO 2004/052846) under the conditions of Example 1(a) to give the title compounds as a tan solid. MS (ESI, pos. ion) m/z: 401 [M+1].

### EXAMPLE 33

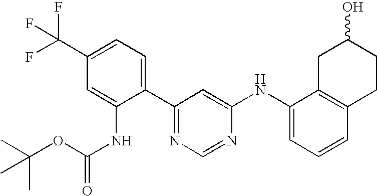

### tert-Butyl 2-(6-(7-hydroxy-5,6,7,8-tetrahydronaphthalen-1-ylamino)-pyrimidine-4-yl)-5-(trifluoromethyl)phenylcarbamate

The title compound was formed as a side product of the reaction described in Example 32, and was isolated as tan solid. MS (ESI, pos. ion) m/z: 501 [M+1].

### EXAMPLE 34

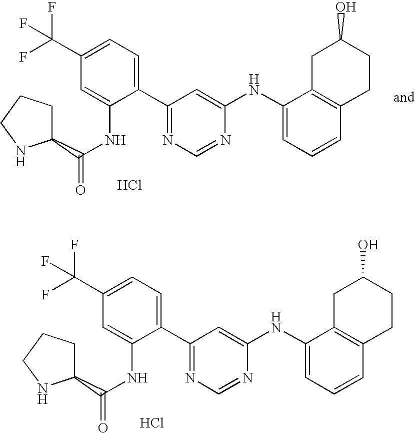

### (2S)-N-(2-(6-((7S)-7-Hydroxy-5,6,7,8-tetrahydronaphthalen-1-ylamino)pyrimidin-4-yl)-5-(trifluoromethyl)phenyl)pyrrolidine-2-carboxamide hydrochloride and (2S)-N-(2-(6-((7R)-7-Hydroxy-5,6,7,8-tetrahydro-naphthalen-1-ylamino)pyrimidin-4-yl)-5-(trifluoromethyl)phenyl)-pyrrolidine-2-carboxamide hydrochloride

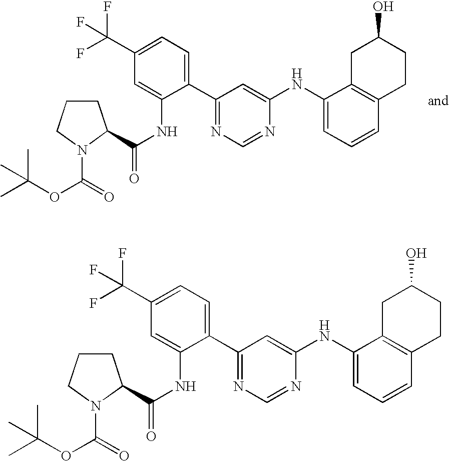 (a) (2S)-tert-Butyl 2-((2-(6-((7S)-7-hydroxy-5,6,7,8-tetrahydro-naphthalen-1-ylamino)pyrimidin-4-yl)-5-(trifluoromethyl)phenyl)-carbamoyl)-pyrrolidine-1-carboxylate and (2S)-tert-Butyl 2-((2-(6-((7R)-7-hydroxy-5,6,7,8-tetrahydro-naphthalen-1-ylamino)pyrimidin-4-yl)-5-(trifluoromethyl)-phenyl)-carbamoyl)-pyrrolidine-1-carboxylate.

A mixture of 8-(6-(2-amino-4-(trifluoromethyl)phenyl)pyrimidin-4-ylamino)-1,2,3,4-tetrahydronaphthaen-2-ol, Example 32, (0.20 g, 0.50 mmol), N-tert-butoxycarbonyl)-L-proline (0.11 g, 0.50 mmol, Aldrich), 1-(3-(dimethylamino)propyl)-3-ethylcarbodiimide hydrochloride (0.10 g, 0.50 mmol, Aldrich) in THF (5 mL) was stirred at 40° C. for 18 h. To the reaction mixture was added N-tert-butoxycarbonyl)-L-proline (0.05 g, 0.25 mmol, Aldrich) and 1-(3-(dimethylamino)propyl)-3-ethylcarbodiimide hydrochloride (0.05 g, 0.25 mmol, Aldrich). Stirring was continued at 60° C. for 3 h. The reaction mixture was evaporated under reduced pressure and the residue was dissolved in EtOAc. The EtOAc solution was washed with satd aqueous solution of Na₂CO₃, dried over Na₂SO₄, and filtered. The filtrate was evaporated under reduced pressure and the residue was purified by silica gel column chromatography (20% EtOAc/hexane) to give the title compound as a mixture of diastereoisomers. MS (ESI, pos. ion) m/z: 597 [M+1]. 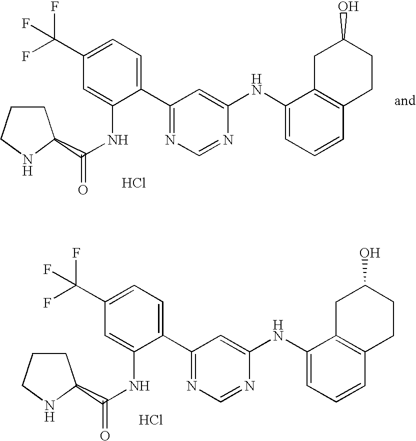
(b) (2S)-N-(2-(6-((7S)-7-Hydroxy-5,6,7,8-tetrahydronaphthalen-1-ylamino)pyrimidin-4-yl)-5-(trifluoromethyl)phenyl)pyrrolidine-2-carboxamide hydrochloride and (2S)-N-(2-(6-((7R)-7-Hydroxy-5,6,7,8-tetrahydro-naphthalen-1-ylamino)pyrimidin-4-yl)-5-(trifluoromethyl)phenyl)-pyrrolidine-2-carboxamide hydrochloride.

A mixture of (2S)-tert-Butyl 2-((2-(6-((7S)-7-hydroxy-5,6,7,8-tetrahydro-naphthalen-1-ylamino)pyrimidin-4-yl)-5-(trifluoromethyl)phenyl)-carbamoyl)-pyrrolidine-1-carboxylate and (2S)-tert-Butyl 2-((2-(6-((7R)-7-hydroxy-5,6,7,8-tetrahydro-naphthalen-1-ylamino)-pyrimidin-4-yl)-5-(trifluoromethyl)phenyl)-carbamoyl)-pyrrolidine-1-carboxylate from step (a) above (0.15 g, 0.25 mmol) and 4 M hydrogen chloride in dioxane (10 mL, Aldrich) was stirred at room temperature for 18 h. The reaction mixture was evaporated under reduced pressure and dried in vacuo to give the title compound as a mixture of diastereoisomers. MS (ESI, pos. ion) m/z: 497 [M+1].

### ADDITIONAL EXAMPLES

TABLE 2
The following examples were prepared from 8-(6-(2-amino-4-
(trifluoromethyl)phenyl) pyrimidin-4-ylamino)-1,2,3,4-tetrahydronaphthalen-2-ol.
(Example 32) and commercially available reagents by following the procedures
described in Example 34(a), or with slight modifications thereof:

| | | MS* | Melting Point |
|---|---|---|---|
| Example | Structure | m/z | ° C. |
| 35 | 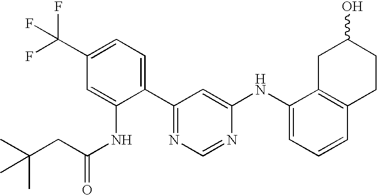 | 499 | White amorphous. solid |
| 36 | 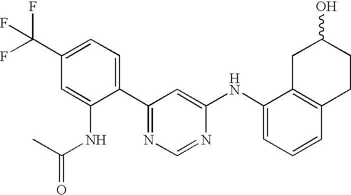 | 443 | Tan amorphous. solid |

*(ESI, pos. ion)

### EXAMPLE 37

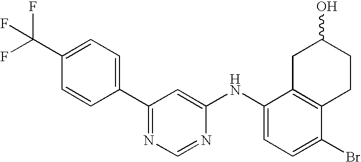

### 5-Bromo-8-(6-(4-(trifluoromethyl)phenyl)pyrimidin-4-ylamino)-1,2,3,4-tetrahydronaphthalen-2-ol

A mixture of 8-(6-(4-(trifluoromethyl)phenyl)-pyrimidin-4-ylamino)-1,2,3,4-tetrahydronaphthalen-2-ol, Example 1(b), (0.77 g, 2.0 mmol) and N-bromosuccinimide (0.43 g, 2.4 mmol, Aldrich) in DMF (10 mL) was stirred at room temperature for 1 h. The reaction mixture was evaporated under reduced pressure and the residue was purified by silica gel column chromatography (40% ethyl acetate/hexane) to give the title compound as an off-white solid. M.p.: 192° C. MS (ESI, pos. ion) m/z: 464, 466[M+1, M+3].

### EXAMPLES 38

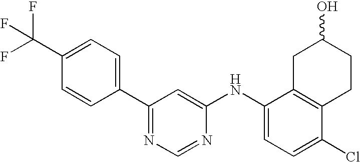

### 5-Chloro-8-(5-chloro-6-(4-(trifluoromethyl)phenyl)pyrimidin-4-ylamino)-1,2,3,4-tetrahydronaphthalen-2-ol

A mixture of 8-(6-(4-(trifluoromethyl)phenyl)pyrimidin-4-ylamino)-1,2,3,4-tetrahydronaphthalen-2-ol, Example 1(b), (1.2 g, 3.0 mmol) and N-chlorosuccinimide (0.48 g, 3.6 mmol, Aldrich) in DMF (30 mL) was stirred at room temperature for 4 h. The reaction mixture was evaporated under reduced pressure and the residue was dissolved in DCM. The DCM solution was washed with 1 N NaOH, dried over Na₂SO₄, and filtered. The filtrate was evaporated under reduced pressure and the residue was purified by silica gel column chromatography (gradient: 30-50% EtOAc/hexane) to give the title compound as a white amorphous solid. MS (ESI, pos. ion) m/z: 454 [M+1].

### EXAMPLE 39 AND EXAMPLE 40

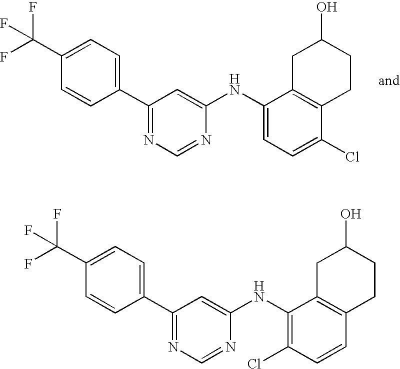

### 5-Chloro-8-(6-(4-(trifluoromethyl)phenyl)pyrimidin-4-ylamino)-1,2,3,4-tetrahydronaphthalen-2-ol and 7-Chloro-8-(6-(4-(trifluoromethyl)phenyl)-pyrimidin-4-ylamino)-1,2,3,4-tetrahydronaphthalen-2-ol

The title compounds were formed as side products of the reaction described in Example 38 and isolated as a mixture after the purification by column chromatography. Separation of the mixture by reverse phase preparative HPLC [Synergi MaxRP column; eluent: 0.1% TFA in H₂O/CH₃CN] afforded the title compounds under the form of TFA salts. Each individual salt was dissolved in chloroform, washed with 10% Na₂CO₃, dried over Na₂SO₄, and filtered. The filtrate was evaporated under reduced pressure and the residue dried under vacuo to give 5-chloro-8-(6-(4-(trifluoromethyl)phenyl)pyrimidin-4-ylamino)-1,2,3,4-tetrahydronaphthalen-2-ol, Example 39, [MS (ESI, pos. ion) m/z: 420 [M+1]] and 7-chloro-8-(6-(4-(trifluoro-methyl)phenyl)pyrimidin-4-ylamino)-1,2,3,4-tetrahydronaphthalen-2-ol, Example 40, [MS (ESI, pos. ion) m/z: 420 [M+1]] correspondingly, as amorphous white solids.

### EXAMPLE 41 AND EXAMPLE 42

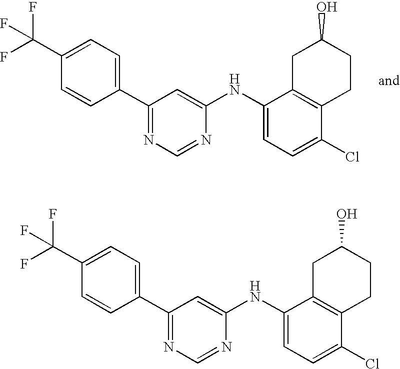

### (S)-5-Chloro-8-(6-(4-(trifluoromethyl)phenyl)pyrimidin-4-ylamino)-1,2,3,4-tetrahydronaphthalen-2-ol and (R)-5-Chloro-8-(6-(4-(trifluoromethyl)-phenyl)pyrimidin-4-ylamino)-1,2,3,4-tetrahydronaphthalen-2-ol

The racemic mixture of 5-chloro-8-(6-(4-(trifluoromethyl)phenyl)pyrimidin-4-ylamino)-1,2,3,4-tetrahydronaphthalen-2-ol (Example 39) was resolved on a chiral stationary phase [Chiralpak AD-H column] by preparative super-critical fluid chromatography [60:40 CO₂(1):0.2% diethylamine in EtOH]. The fractions containing product were evaporated under reduced pressure and the residue was dissolved in chloroform. The chloroform solution was washed with 10% Na₂CO₃, dried over Na₂SO₄, and filtered. The filtrate was evaporated under reduced pressure and the residue dried in vacuo to give the title compounds as white amorphous solids. MS (ESI, pos. ion) m/z: 420 [M+1].

### EXAMPLE 43

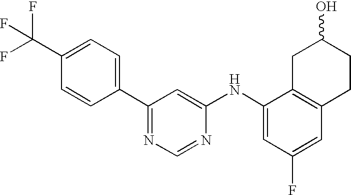

### 6-Fluoro-8-(6-(4-(trifluoromethyl)phenyl)pyrimidin-4-ylamino)-1,2,3,4-tetrahydronaphthalen-2-ol

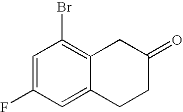 (a) 8-Bromo-6-fluoro-3,4-dihydronaphthalen-2(1H)-one.

[Analogous to the method of Stjernlof, P.; Ennis, M. D.; Hansson, L. O.; Hoffman, R. L.; Ghazal, N. B.; Sundell, S.; Smith, M. W.; Svensson, K.; Carlsson, A.; Wikstrom, H. Structure-activity relationships in the 8-amino-6,7,8,9-tetrahydro-3H-benz[e]indole ring system. 1. Effects of substituents in the aromatic system on serotonin and dopamine receptor subtypes. J. Med. Chem. 1995, 38(12), 2202-16].

A mixture of 2-bromo-4-fluorophenylacetic acid (2.3 g, 10 mmol, Aldrich) and thionyl chloride (2.6 g, 22 mmol, Aldrich) in DCM (30 mL) was heated at 50° C. with stirring for 3 h. The reaction mixture was allowed to reach room temperature and was evaporated under reduced pressure. The residue was dissolved in dichloromethane (10 mL) and the solution was added dropwise to a stirred suspension of aluminum chloride (4.0 g, 30 mmol, Aldrich) in CH₂Cl₂ (100 mL) at −5° C. Ethylene gas was bubbled through the reaction mixture for 1 h at −5° C. The reaction mixture was poured onto a mixture of ice and concd HCl. The organic layer was separated, washed with 10% Na₂CO₃, dried over Na₂SO₄, and filtered. The filtrate was evaporated under reduced pressure and the residue was purified by silica gel column chromatography (gradient: 5-10% EtOAc/hexane) to give the title compound as a white solid. MS (ESI, pos. ion) m/z: 243, 245 [M+1, M+3]. 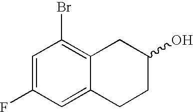
(b) 8-Bromo-6-fluoro-1,2,3,4-tetrahydronaphthalen-2-ol.

A solution of 8-bromo-6-fluoro-3,4-dihydronaphthalen-2(1H)-one from step (a) above (1.3 g, 5.3 mmol) in MeOH (20 mL) was treated with sodium borohydride (0.23 g, 6.0 mmol, Aldrich) and stirred at room temperature for 3 h. The reaction mixture was evaporated under reduced pressure and the residue dissolved in DCM. The DCM solution was washed with 1 N NaOH, dried over Na₂SO₄, and filtered. The filtrate was evaporated under reduced pressure and the residue was purified by silica gel column chromatography (10% EtOAc/hexane) to give the title compound as a white solid. MS (ESI, pos. ion) m/z: 227, 229 [M-OH, M+2-OH]. 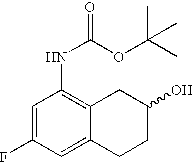
(c) tert-Butyl 3-fluoro-7-hydroxy-5,6,7,8-tetrahydronaphthalen-1-yl-carbamate.

To a round-bottomed flask, under N₂, was added 8-bromo-6-fluoro-1,2,3,4-tetrahydronaphthalen-2-ol from step (b) above (0.49 g, 2.0 mmol), tert-butylcarbamate (0.26 g, 2.8 mmol, Aldrich), cesium carbonate 0.92 g, 2.8 mmol, Aldrich), tris(dibenzylydeneacetone)dipalladium (0.050 g, 2.5 mol %, Aldrich), 2-dicyclohexylphosphino-2′,4′,6′-triisopropyl-1,1′-biphenyl (0.050 g, 5 mol %, Strem), and dioxane (5 mL). The reaction mixture was stirred at 90° C. under N₂ in an oil bath for 18 h. The reaction mixture was concentrated, dissolved in ethyl acetate and filtered through a Celite® pad. The filtrate was washed with 10% Na₂CO₃, dried over Na₂SO₄, and filtered. The filtrate was evaporated under reduced pressure and the residue was purified by silica gel column chromatography (gradient: 20-30% EtOAc/hexane) to give the title compound as a syrup. MS (ESI, pos. ion) m/z: 282 [M+1]. 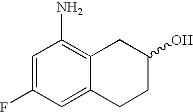
(d) 8-Amino-6-fluoro-1,2,3,4-tetrahydronaphthalen-2-ol.

A mixture of tert-butyl 3-fluoro-7-hydroxy-5,6,7,8-tetrahydronaphthalen-1-ylcarbamate from step (c) above (0.13 g, 0.46 mmol) and 4 M hydrogen chloride in dioxane (5 mL, Aldrich) was stirred at room temperature for 3 h. The reaction mixture was evaporated under reduced pressure and the residue was dissolved in DCM. The DCM solution was washed with 10% Na₂CO₃, dried over Na₂SO₄, and filtered. The filtrate was evaporated under reduced pressure and the residue was purified by silica gel column chromatography (gradient: 50-80% EtOAc/hexane) to give the title compound as a syrup. MS (ESI, pos. ion) m/z: 182 [M+1]. 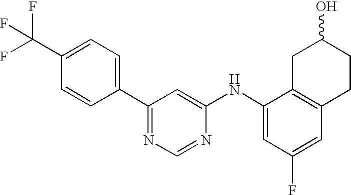
(e) 6-Fluoro-8-(6-(4-(trifluoromethyl)phenyl)pyrimidin-4-ylamino)-1,2,3,4-tetrahydronaphthalen-2-ol.

4-Chloro-6-(4-(trifluoromethyl)phenyl)pyrimidine,

Example 1(a), (0.10 g, 0.40 mmol) was reacted with 8-amino-6-fluoro-1,2,3,4-tetrahydronaphthalen-2-ol from step (e) above (0.04 g, 0.20 mmol) under the conditions of Example 1(a) to give the title compound as an off-white amorphous solid. MS (ESI, pos. ion) m/z: 404 [M+1].

### EXAMPLE 44

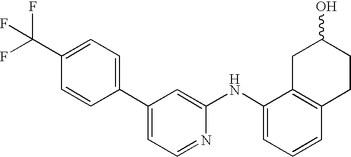

### 8-(4-(4-(Trifluoromethyl)phenyl)pyridin-2-ylamino)-1,2,3,4-tetrahydro-naphthalen-2-ol

2-Chloro-4-(4-(trifluoromethyl)phenyl)pyridine (0.52 g, 2.0 mmol, prepared as described in WO 2003/049702), 8-amino-1,2,3,4-tetrahydro-naphthalen-2-ol (0.33 g, 2.0 mmol, prepared analogously to WO 2004/052846), rac-2,2-bis(diphenylphosphino)-1,1-binaphthyl (0.12 ml, 0.20 mmol, Aldrich) and palladium acetate (0.023 g, 0.10 mmol, Aldrich) were combined in toluene (5 mL) under nitrogen atmosphere. Sodium tert-butoxide (0.27 g, 2.8 mmol, Aldrich) was added, and the mixture was stirred at 90° C. for 1 h. The reaction mixture was evaporated under reduced pressure and the residue was dissolved in chloroform. The chloroform solution was washed with 10% Na₂CO₃, dried over Na₂SO₄, and filtered. The filtrate was evaporated under reduced pressure and the residue was purified by silica gel column chromatography (70% EtOAc/hexane), followed by reverse-phase preparative HPLC [Phenomenex Gemini column; mobile phase: 0.1% TFA in CH₃CN/H₂O]. The fractions containing product were evaporated under reduced pressure. The residue was dissolved in chloroform, washed with 10% Na₂CO₃, dried over Na₂SO₄, and filtered. The filtrate was evaporated under reduced pressure and the residue dried in vacuo to give the title compound as a white amorphous solid. MS (ESI, pos. ion) m/z: 385 [M+1].

### EXAMPLE 45

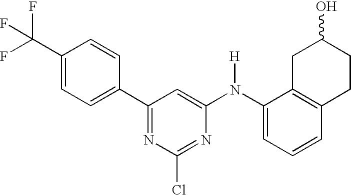

### 8-(2-Chloro-6-(4-(trifluoromethyl)phenyl)pyrimidin-4-ylamino)-1,2,3,4-tetrahydronaphthalen-2-ol

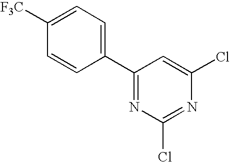 (a) 2,4-Dichloro-6-(4-trifluoromethyl-phenyl)-pyrimidine.

A mixture of 2,4,6-trichloropyrimidine (502 mg, 2.74 mmol, Aldrich), 4-trifluoromethyl-phenylboronic acid (510 mg, 2.69 mmol, Aldrich), Pd(OAc)₂ (31 mg, 0.14 mmol), PPh₃ (71 mg, 0.27 mmol, Aldrich) and 2 N Na₂CO₃ (3.1 mL) in 1,2-dimethoxyethane (9.0 mL) was heated at 120° C. in a microwave synthesizer for 20 min. The reaction mixture was cooled to room temperature, diluted with saturated aqueous solution of NaHCO₃ (10 mL) and extracted with EtOAc (2×50) mL. The combined EtOAc extracts were dried over Na₂SO₄, filtered, and concentrated in vacuo. Purification of the residue by silica gel column chromatography (gradient 3-12% EtOAc/hexane) gave the title compound as an off-white solid. MS (ESI, pos. ion.) m/z: 294 [M+1]. 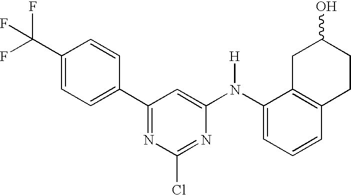
(b) 8-(2-Chloro-6-(4-(trifluoromethyl)phenyl)pyrimidin-4-ylamino)-1,2,3,4-tetrahydronaphthalen-2-ol.

A mixture of 2,4-dichloro-6-(4-trifluoromethyl-phenyl)-pyrimidine from step (a) above (293 mg, 1.0 mmol) and 8-amino-1,2,3,4-tetrahydronaphthalen-2-ol (163 mg, 1.0 mmol, prepared analogously to WO 2004/052846) in EtOH. (5 mL) was heated at 70° C. for 16 h. The reaction mixture was allowed to reach room temperature and evaporated under reduced pressure. The residue was purified by silica gel column chromatography (40% EtOAc/hexane) to give the title compound as a white amorphous solid. MS (ESI, pos. ion) m/z: 420 [M+1].

### EXAMPLE 46

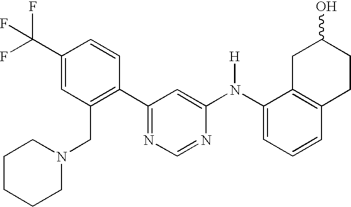

### 8-(6-(2-(Piperidin-1-ylmethyl)-4-(trifluoromethyl)phenyl)pyrimidin-4-ylamino)-1,2,3,4-tetrahydronaphthalen-2-ol

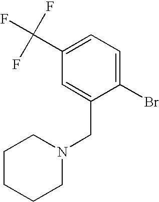 (a) 1-(2-Bromo-5-(trifluoromethyl)benzyl)piperidine.

A mixture of 2-bromo-5-(trifluoromethyl)benzaldehyde (5.1 g, 20 mmol, prepared according to the procedure described in J. Med. Chem. 1986, 29, 1142-1452.) and piperidine (2.2 g, 25 mmol, Aldrich) in chloroform (20 mL) was stirred at room temperature for 10 min, and treated with NaBH(OAc)₃ (6.3 g, 30 mmol, Aldrich). The reaction mixture was stirred at room temperature for 1 h, washed with 1 N NaOH solution (40 mL) and brine, dried over Na₂SO₄, and filtered. The filtrate was evaporated under reduced pressure and the residue purified by silica gel column chromatography (6% MeOH/DCM) to give the title compound as a light-yellow oil. MS (ESI, pos. ion) m/z: 322 [M+1]. 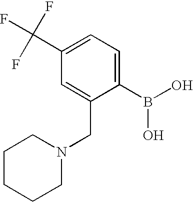
(b) 2-(Piperidin-1-ylmethyl)-4-(trifluoromethyl)phenylboronic acid.

A solution of 1-(2-bromo-5-(trifluoromethyl)benzyl)piperidine from step (a) above (1.93 g, 6 mmol) in anhydrous THF (8 mL) was added dropwise to a stirred suspension of highly reactive Rieke® magnesium in THF [7.6 mL, 12 mmol, (2.5 g Mg in 100 mL THF), Rieke] at 0° C. under nitrogen atmosphere. The reaction mixture was heated to reflux for 3 h and allowed to reach room temperature. The reaction mixture was cooled to −60° C. and trimethylborate (1.35 mL, 12 mmol, Aldrich) was added dropwise. After the addition, the mixture was stirred for 1 h at −60° C., and then allowed to warm gradually to room temperature overnight. The reaction mixture was quenched with satd aqueous solution of NH₄Cl (25 mL) and exacted with ether (150 mL). The organic extract was separated, dried over Na₂SO₄, and filtered. The filtrate was evaporated under reduced pressure and the residue dried under vacuo to give the title compound as an amorphous solid. MS (ESI, pos. ion) m/z: 288 [M+1]. 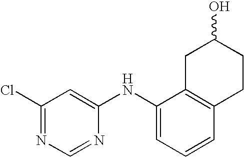
(c) 8-(6-Chloropyrimidin-4-ylamino)-1,2,3,4-tetrahydronaphthalen-2-ol.

A mixture of 4,6-dichloropyrimidine (149 mg, 1.0 mmol, Aldrich), 8-amino-1,2,3,4-tetrahydronaphthalen-2-ol (163 mg, 1.0 mmol, prepared analogously to WO 2004/052846) and cesium carbonate (326 mg, 1.0 mmol) in DMF (6 mL) was heated at 120° C. with stirring for 16 h. The reaction mixture was allowed to cool to room temperature, diluted with water (20 mL), and extracted with EtOAc (60 mL). The organic extract was washed with water and brine, dried over Na₂SO₄, and filtered. The filtrate was evaporated under reduced pressure and the residue purified by silica gel column chromatography (50% EtOAc/hexane) to give the title compound as an amorphous solid. MS (ESI, pos. ion) m/z: 274 [M+1]. 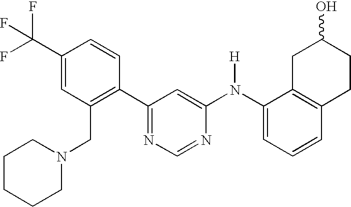
(d) 8-(6-(2-(Piperidin-1-ylmethyl)-4-(trifluoromethyl)phenyl)pyrimidin-4-ylamino)-1,2,3,4-tetrahydronaphthalen-2-ol.

A mixture of 2-(piperidin-1-ylmethyl)-4-(trifluoromethyl)phenylboronic acid from step (b) above (72 mg, 0.25 mmol), 8-(6-chloropyrimidin-4-ylamino)-1,2,3,4-tetrahydronaphthalen-2-ol from step (c) above (69 mg, 0.25 mmol), PdCl₂(PPh₃)₂ (35 mg, 0.05 mmol, Aldrich), Na₂CO₃.H₂O (62 mg, 0.5 mmol), dimethoxyethane (0.7 mL), H₂O (0.3 mL) and EtOH (0.2 mL) was heated in a microwave synthesizer at 120° C. for 10 min. Water (5 mL) was added, and the mixture was extracted with EtOAc (2×10 mL). The combined organic extracts were washed with brine (10 mL), dried over Na₂SO₄, and filtered. The filtrate was evaporated under reduced pressure and the residue purified by silica gel column chromatography (7% MeOH/hexane) to give the title compound as amorphous solid. MS (ESI, pos. ion) m/z: 483 [M+1].

### EXAMPLE 47

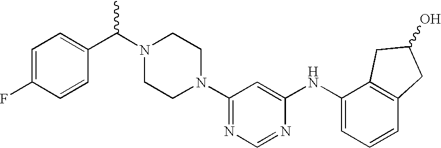

### (2R,S)-4-(6-(4-((R,S)-1-(4-Fluorophenyl)ethyl)piperazin-1-yl)pyrimidin-4-ylamino)-2,3-dihydro-1H-inden-2-ol

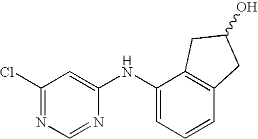 (a) 4-(6-Chloropyrimidin-4-ylamino)-2,3-dihydro-1H-inden-2-ol.

A mixture of 4,6-dichloropyrimidine (150 mg, 1.0 mmol, Aldrich), 4-amino-2,3-dihydro-1H-inden-2-ol, Example 27(c), (150 mg, 1 mmol) and PS-DIEA (500 mg, 1.8 mmol, Argonaut Technologies) in EtOH (4 mL) was heated in a microwave synthesizer at 150° C. for 5 min. The reaction mixture was filtered and the filter cake was washed with MeOH. The combined filtrates were evaporated under reduced pressure and the residue purified by silica gel column chromatography (gradient: 2-10% MeOH/DCM) to give the title compound as a white amorphous solid. MS (ESI, pos. ion) m/z: 262 [M+1]. 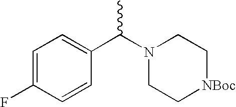
(b) 4-[1-(4-Fluoro-phenyl)-ethyl]-piperazine-1-carboxylic acid tert-butyl ester.

To a solution of piperazine-1-carboxylic acid tert-butyl ester (0.39 g, 2.2 mmol, Fluka) and 4-fluoro-acetophenone (0.39 mL, 3.3 mmol, Aldrich) in THF (2 mL) was added titanium(IV) isopropoxide (1.9 mL, 6.6 mmol, Aldrich) and the mixture was stirred at 75° C. for 18 h under nitrogen atmosphere. The reaction mixture was cooled to −48° C. and treated with NaBH(OAc)₃ (1.23 g, 6.44 mmol, Aldrich) and methanol (1 mL). The mixture was allowed to warm to room temperature over 3.5 h and diluted with EtOAc (100 mL). The EtOAc solution was washed with 1N NaOH (3×100 mL). The organic layer was separated, dried over Na₂SO₄, filtered, and concentrated in vacuo. The residue was purified by silica gel column chromatography (gradient: 0-4% MeOH/CH₂Cl₂) to give the title compound as a yellow oil. MS (ESI, pos. ion.) m/z: 309.2 [M+1]. 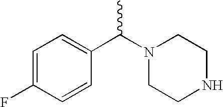
(c) 1-[1-(4-Fluoro-phenyl)-ethyl]-piperazine.

To a solution of 4-[1-(4-fluoro-phenyl)-ethyl]-piperazine-1-carboxylic acid tert-butyl ester, from step (b) above (0.42 g, 1.36 mmol) in CH₂Cl₂ (5 mL) was added TFA (0.5 mL, 6.5 mmol, Aldrich) dropwise with stirring at 0° C. The reaction mixture was stirred at room temperature for 18 h and evaporated under reduced pressure. The residue was dried in vacuo to give the crude title compound, which was used in the next step without additional purification. 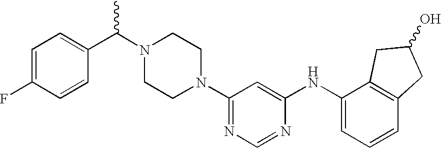
(d) (2R,S)-4-(6-(4-((R,S)-1-(4-Fluorophenyl)ethyl)piperazin-1-yl)pyrimidin-4-ylamino)-2,3-dihydro-1H-inden-2-ol.

A mixture of 4-(6-chloropyrimidin-4-ylamino)-2,3-dihydro-1H-inden-2-ol from step (a) above (95 mg, 0.36 mmol) and 1-[1-(4-fluoro-phenyl)-ethyl]-piperazine from step (c) above (85 mg, 0.4 mmol) in EtOH (3 mL) was heated in a microwave synthesizer at 150° C. for 5 min. The reaction mixture was evaporated under reduced pressure and the residue purified by silica gel column chromatography (gradient: 5-10% MeOH/DCM) to give the title compound as a mixture of diastereoisomers. MS (ESI, pos. ion) m/z: 434 [M+1].

### EXAMPLE 48

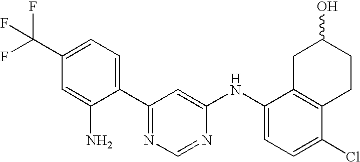

### 8-(6-(2-Amino-4-(trifluoromethyl)phenyl)pyrimidin-4-ylamino)-5-chloro-1,2,3,4-tetrahydronaphthalen-2-ol

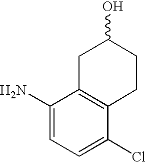 (a) 8-Amino-5-chloro-1,2,3,4-tetrahydronaphthalen-2-ol.

A mixture of 8-amino-1,2,3,4-tetrahydronaphthalen-2-ol (0.98 g, 6.0 mmol, prepared analogously to WO 2004/052846) and N-chlorosuccinimide (0.88 g, 6.6 mmol, Aldrich) in DMF (30 mL) was stirred at room temperature for 3 h. The reaction mixture was evaporated under reduced pressure and the residue was dissolved in EtOAc (30 mL). The EtOAc solution was washed with 10% Na₂CO₃, dried over Na₂SO₄, and filtered. The filtrate was evaporated under reduced pressure and the residue purified by silica gel column chromatography (gradient: 40-80% EtOAc/hexane) to give the title compound as an amorphous solid. MS (ESI, pos. ion) m/z: 198 [M+1]. 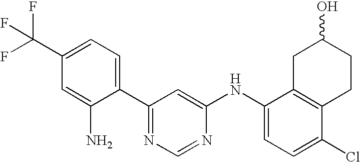
(b) 8-(6-(2-Amino-4-(trifluoromethyl)phenyl)pyrimidin-4-ylamino)-5-chloro-1,2,3,4-tetrahydronaphthalen-2-ol.

8-Amino-5-chloro-1,2,3,4-tetrahydronaphthalen-2-ol from step (a) above (0.40 g, 2 mmol) was reacted with tert-butyl 2-(6-chloropyrimidin-4-yl)-5-(trifluoromethyl)phenylcarbamate (0.75 g, 2 mmol, prepared according to WO 2004/014871) under the conditions of Example 1(b) to give the title product as tan amorphous solid. MS (ESI, pos. ion) m/z: 435 [M+1].

### EXAMPLE 49

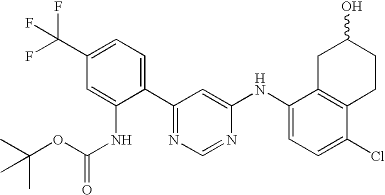

### tert-Butyl 2-(6-(4-chloro-7-hydroxy-5,6,7,8-tetrahydronaphthalen-1-ylamino)-pyrimidin-4-yl)-5-(trifluoromethyl)phenylcarbamate

The title compound was formed as a side product of the reaction described in Example 48(b), and isolated as tan amorphous solid. MS (ESI, pos. ion) m/z: 535 [M+1].

### EXAMPLE 50

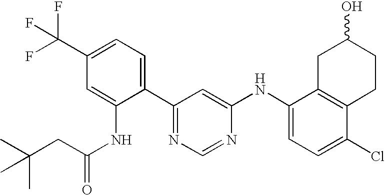

### N-(2-(6-(4-Chloro-7-hydroxy-5,6,7,8-tetrahydronaphthalen-1-ylamino)-pyrimidin-4-yl)-5-(trifluoromethyl)phenyl)-3,3-dimethylbutanamide

A mixture of 8-(6-(2-amino-4-(trifluoromethyl)phenyl)pyrimidin-4-ylamino)-5-chloro-1,2,3,4-tetrahydronaphthalen-2-ol, Example 48(b), (0.2 g, 0.5 mmol), N,N-diisopropylethylamine (0.06 g, 0.5 mmol, Aldrich) and tert-butylacetyl chloride (0.2 g, 2 mmol, Aldrich) in DCM (5 mL) was stirred at room temperature for 16 h. The reaction mixture was evaporated under reduced pressure and the residue was dissolved in MeOH. To the MeOH solution was added 1 N NaOH (1 mL) and the mixture was heated at 40° C. for 1 h. The reaction mixture was evaporated under reduced pressure and the residue was dissolved in DCM. The DCM solution was washed with satd NaHCO₃ (50 mL), dried over Na₂SO₄, filtered and evaporated under reduced pressure. The residue was purified by reverse-phase preparative HPLC [Phenomenex Gemini column, 0.1% TFA in CH₃CN/H₂O]. The fractions containing product were combined and concentrated under reduced pressure to remove CH₃CN. The resulting aqueous mixture was treated with satd NaHCO₃ and extracted with CHCl₃. The organic extract was washed with brine, dried over Na₂SO₄, and filtered. The filtrate was evaporated under reduced pressure and the residue was dried in vacuo to afford the title product as a white amorphous solid. MS (ESI, pos. ion) m/z: 533 [M+1].

### EXAMPLE 51

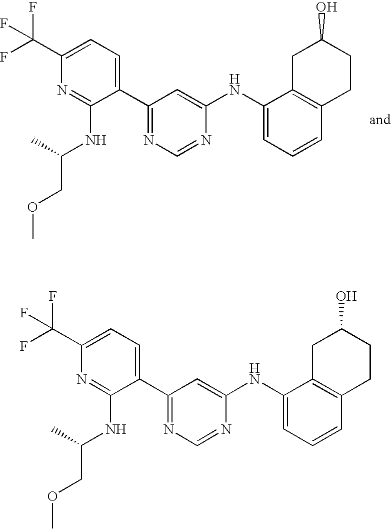

### (2S)-8-(6-(2-((2S)-1-methoxypropan-2-ylamino)-6-(trifluoromethyl)pyridin-3-yl)-pyrimidin-4-ylamino)-1,2,3,4-tetrahydronaphthalen-2-ol and (2R)-8-(6-(2-((2S)-1-methoxypropan-2-ylamino)-6-(trifluoromethyl)pyridin-3-yl)-pyrimidin-4-ylamino)-1,2,3,4-tetrahydronaphthalen-2-ol

A mixture of 8-(6-(2-chloro-6-(trifluoromethyl)pyridin-3-yl)pyrimidin-4-ylamino)-1,2,3,4-tetrahydronaphthalen-2-ol, Example 5(c), (0.17 g, 0.4 mmol) and (S)-1-methoxypropan-2-amine (0.1 g, 1 mmol) in DMSO (2 mL) was heated at 100° C. with stirring for 18 h. The reaction mixture was allowed to reach room temperature, and was diluted with EtOAc. The EtOAc solution was washed with brine, dried over Na₂SO₄, and filtered. The filtrate was evaporated under reduced pressure and the residue was purified by reverse-phase preparative HPLC [Phenomenex Gemini column, 0.1% TFA in CH₃CN/H₂O]. The fractions containing product were combined and concentrated under reduced pressure to remove CH₃CN. The resulting aqueous mixture was treated with satd NaHCO₃ and extracted with CHCl₃. The organic extract was washed with brine, dried over Na₂SO₄, and filtered. The filtrate was evaporated under reduced pressure and the residue was dried in vacuo to afford the title product as an amorphous solid. MS (ESI, pos. ion) m/z: 508 [M+1].

### EXAMPLE 52

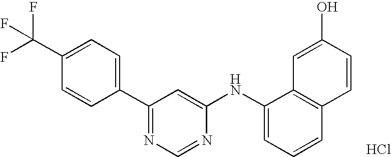

### 8-(6-(4-(Trifluoromethyl)phenyl)pyrimidin-4-ylamino)naphthalen-2-ol hydrochloride

4-Chloro-6-[4-(trifluoromethyl)phenyl]pyrimidine, Example 1(a), (0.36 g, 1.4 mmol) was reacted with 8-amino-2-naphthol (0.20 mg, 1.25 mmol, Aldrich) under the conditions of Example 1(b) to give the title compound as a yellow-green solid. MS (ESI, pos. ion) m/z: 381[M+1].

### EXAMPLE 53

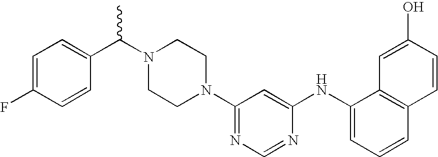

### 8-(6-(4-(1-(4-Fluorophenyl)ethyl)piperazin-1-yl)pyrimidin-4-ylamino)-naphthalen-2-ol

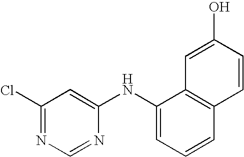 (a) 8-(6-Chloropyrimidin-4-ylamino)naphthalen-2-ol.

A mixture of 4,6-dichloropyrimidine (0.476 g, 3.19 mmol, Aldrich), 8-amino-2-naphthol (0.507 g, 3.18 mmol, Aldrich) and N,N-diisopropylethylamine (1 mL, Aldrich) in EtOH (3 mL) was heated in a microwave synthesizer at 110° C. for 6 min. The reaction mixture was evaporated under reduced pressure and the residue purified by silica gel column chromatography (gradient: 2-10% MeOH/DCM) to give the title compound as a white amorphous solid. MS (ESI, pos. ion) m/z: 272 [M+1]. 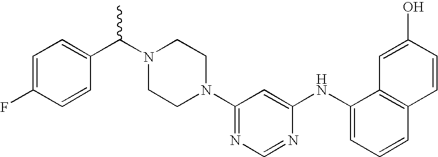
(b) 8-(6-(4-(1-(4-Fluorophenyl)ethyl)piperazin-1-yl)pyrimidin-4-ylamino)-naphthalen-2-ol.

A mixture of 8-(6-chloropyrimidin-4-ylamino)naphthalen-2-ol from step (a) above (0.342 g, 1.26 mmol), 1-[1-(4-fluoro-phenyl)-ethyl]-piperazine, Example 47(c), (85 mg, 0.4 mmol) and N,N-diisopropylethylamine (1 mL, Aldrich) in EtOH (4 mL) was heated in a microwave synthesizer at 160° C. for 6 min. The reaction mixture was evaporated under reduced pressure and the residue purified by silica gel column chromatography (gradient: 5-10% MeOH/DCM) to give the title compound as a white amorphous solid. MS (ESI, pos. ion) m/z: 444 [M+1].

### EXAMPLE 54

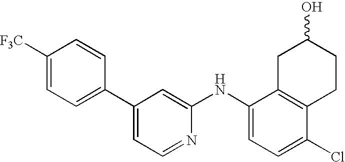

### 5-Chloro-8-(4-(4-(trifluoromethyl)phenyl)pyridin-2-ylamino)-1,2,3,4-tetrahydronaphthalen-2-ol

8-Amino-5-chloro-1,2,3,4-tetrahydronaphthalen-2-ol (0.4 g, 2.0 mmol, Example 48(a)) was reacted with 2-chloro-4-(4-(trifluoromethyl)phenyl)pyridine (0.5 g, 2 mmol, prepared as described in WO 2003/049702) at 90° C. for 1 h under the conditions of Example 44 to give the crude product. Purification of the product by reverse-phase preparative HPLC under the conditions of Example 44 gave the title compound as a white amorphous solid. MS (ESI, pos. ion.) m/z: 419 [M+1].

### EXAMPLE 55

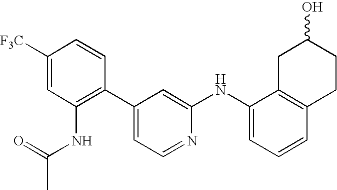

### N-(2-(2-(7-Hydroxy-5,6,7,8-tetrahydronaphthalen-1-ylamino)pyridin-4-yl)-5-(trifluoromethyl)phenyl)acetamide

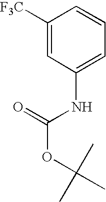 (a) (3-Trifluoromethylphenyl)carbamic acid tert-butyl ester.

To a 250-mL, round-bottomed flask was added 3-(trifluoromethyl)aniline (5.0 g, 31 mmol, Aldrich), THF (100 mL), di-tert-butyl dicarbonate (20.3 g, 93 mmol, Aldrich) and 4-(dimethylamino)pyridine (0.38 g, 3.1 mmol, Aldrich). The mixture was heated at reflux for 3 h. K₂CO₃ (13 g, 93 mmol) and MeOH (50 mL) were added, and heating was continued for 18 h. After cooling to room temperature, the mixture was diluted with CH₂Cl₂, then filtered and washed with CH₂Cl₂. The filtrate was concentrated to afford a brown oil. The oil was dissolved in EtOAc (200 mL) and washed with H₂O (2×100 mL), brine (1×100 mL), dried over Na₂SO₄, filtered and concentrated in vacuum onto silica gel. Purification by silica gel chromatography (gradient: 0-15% EtOAc in hexanes) afforded the title compound as a while solid. MS (ESI, neg. ion.) m/z: 260 (M−1). 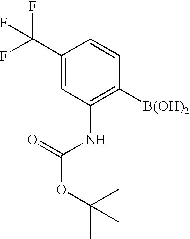
(b) 2-(tert-Butoxycarbonyl)-4-(trifluoromethyl)phenylboronic acid.

(Analogous to the procedures of Boisnard, S.; Carbonnelle, A. C.; Zhu, J. Org. Let. 2001, 3, 2061-2064 and Hewawasam, P.; Meanwell, N. A. Tetrahedron Lett. 1994, 35, 7303). To a 500-mL, round-bottomed flask containing (3-trifluoro-methylphenyl)carbamic acid tert-butyl ester from step (a) above (2.5 g, 9.6 mmol) in THF (100 mL) stirred at 40° C. was added sec-BuLi (17 mL, 1.3 M in cyclohexane, Aldrich) over 10 min. The mixture was stirred for 1 h at −40° C. and then cooled to −78° C. Trimethyl borate (4.4 mL, 38 mmol, Aldrich) was added over 10 min. The reaction mixture was allowed to warm to room temperature and stirred for 10 min at that temperature. The mixture was quenched with aqueous solution of KH₂PO₄ and concentrated to remove the THF. The aqueous mixture was extracted with EtOAc (3×100 mL) and the combined extracts were washed with brine, dried over Na₂SO₄, filtered, and concentrated to afford the title compound as a yellow foam. 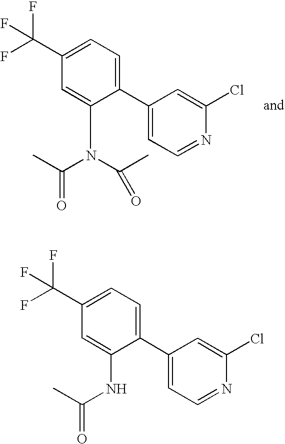
(c) N-(2-(2-Chloropyridin-4-yl)-5-(trifluoromethyl)phenyl)diacetamide and N-(2-(2-Chloropyridin-4-yl)-5-(trifluoromethyl)phenyl)acetamide.

To a 50-mL, round-bottomed flask was added 4-bromo-2-chloropyridine (0.58 g, 3.0 mmol, Asymchem), 2-(tert-butoxycarbonyl)-4-(trifluoromethyl)phenylboronic acid from step (b) above (0.92 g, 3.0 mmol), potassium carbonate (0.83 g, 6.0 mmol, Aldrich), tetrakis(triphenylphosphine)palladium(0) (0.18 g, 0.15 mmol, Strem), toluene (10 mL) and water (0.1 mL). The reaction mixture was heated at 100° C. in an oil bath for 18 h and left to reach room temperature. The mixture was concentrated under reduced pressure, and the residue partitioned between dichloromethane and water. The organic layer was separated, dried over sodium sulfate, filtered, and evaporated. The residue was dissolved in dichloromethane (5 mL), to the solution was added acetic anhydride (0.6 g, 6 mmol, Aldrich) and DMAP (10 mg, Aldrich), and the mixture was heated at 40° C. with stirring for 16 h. The reaction mixture was left to reach room temperature, washed with 10% sodium carbonate, dried over sodium sulfate, and filtered. The filtrate was evaporated under reduced pressure and the residue was purified by silica gel column chromatography eluting with 20% ethyl acetate/hexane to give N-(2-(2-chloropyridin-4-yl)-5-(trifluoromethyl)phenyl)diacetamide. MS (ESI, pos. ion.) m/z: 357 [M+1]. Second elution with 50% ethyl acetate/hexane afforded N-(2-(2-chloropyridin-4-yl)-5-(trifluoromethyl)phenyl)acetamide. MS (ESI, pos. ion.) m/z: 315 [M+1]. 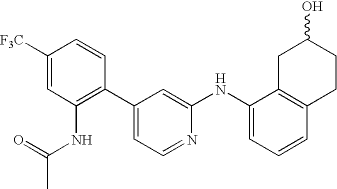
(d) N-(2-(2-(7-Hydroxy-5,6,7,8-tetrahydronaphthalen-1-ylamino)pyridin-4-yl)-5-(trifluoromethyl)phenyl)acetamide.

2-(2-Chloropyridin-4-yl)-5-(trifluoromethyl)phenyl)acetamide from step (c) above (0.21 g, 0.7 mmol) was reacted with 8-amino-1,2,3,4-tetrahydronaphthalen-2-ol (0.1 g, 0.7 mmol, prepared analogously to WO 2004/052846) under the conditions of Example 44 to give the title compound as an amorphous solid. MS (ESI, pos. ion.) m/z: 442 [M+1].

### EXAMPLE 56

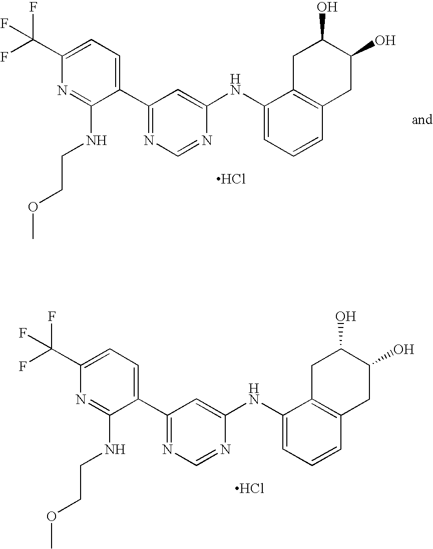

### (2S,3R)-5-(6-(2-(2-Methoxyethylamino)-6-(trifluoromethyl)pyridin-3-yl)pyrimidin-4-ylamino)-1,2,3,4-tetrahydronaphthalene-2,3-diol and (2R,3S)-5-(6-(2-(2-Methoxyethylamino)-6-(trifluoromethyl)pyridin-3-yl)pyrimidin-4-ylamino)-1,2,3,4-tetrahydronaphthalene-2,3-diol, hydrochloride salts

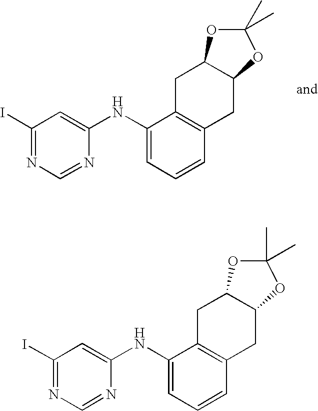 (a) N-((3aR,9aS)-2,2-Dimethyl-3a,4,9,9a-tetrahydronaphthon[2,3-d][1,3]dioxol-5-yl)-6-iodopyrimidin-4-amine and N-((3aS,9aR)-2,2-Dimethyl-3a,4,9,9a-tetrahydronaphtho [2,3-d][1,3]dioxol-5-yl)-6-iodopyrimidin-4-amine.

Analogous to Example 5(a), a mixture of 4,6-diiodopyrimidine (1.2 g, 3.6 mmol, prepared according to Goodman, A. J.; Stanforth, S. P.; Tarbit, B. Desymmetrization of dichloroazaheterocycles. Tetrahedron 1999, 55(52), 15067-15070) and (+)-5-amino-1,2,3,4-tetrahydronaphthalene-2,3-cis-diol (0.76 g crude, Example 30(b)) in NMP (10 mL) was stirred and heated in a Discover® model microwave reactor (CEM, Matthews, N.C.) at 120° C. for 10 min. The crude mixture was transferred to a round-bottomed flask, diluted with acetone (100 mL), treated with p-TsOH monohydrate (50 mg, Aldrich) and stirred at 25° C. for 16 h. Solid K₂CO₃ was added (1 g) and the suspension stirred for 10 min. The solids were removed by filtration and the filtrate concentrated in vacuo to afford a brown solution of crude product in NMP. The solution was diluted with EtOAc (250 mL) then washed with 10% Na₂CO₃ (150 mL), water (2×150 mL), satd NaCl (100 mL), dried over Na₂SO₄, filtered and concentrated in vacuo. The residue was adsorbed onto a plug of silica gel and chromatographed through a Redi-Sep® pre-packed silica gel column (120 g), eluting with a gradient of 30% to 50% EtOAc in hexane, to provide the title product as an off-white foam. MS (ESI, pos. ion) m/z: 424 [M+1]. ₁H NMR (400 MHz, chloroform-d) δ ppm: 1.10 (s, 3 H), 1.30 (s, 3 H), 2.43 (dd, J=3.13, 15.45 Hz, 1 H), 2.70 (dd, J=3.62, 15.16 Hz, 1 H), 2.99 (dd, J=2.84, 15.16 Hz, 1 H), 3.05 (dd, J=3.03, 15.35 Hz, 1 H), 4.64-4.72 (m, 2 H), 6.74 (s, 1 H), 6.77 (br s, 1 H), 7.15 (d, J=7.63 Hz, 1 H), 7.24 (d, J=13.11 Hz, 1 H), 7.26-7.30 (m, 1 H), 8.27 (s, 1 H). 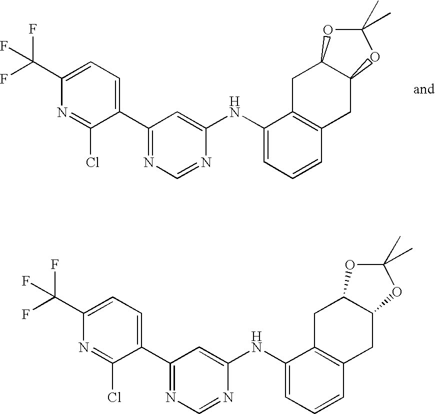
(b) 6-(2-Chloro-6-(trifluoromethyl)pyridin-3-yl)-N-((3aR,9aS)-2,2-dimethyl-3a,4,9,9a-tetrahydronaphtho[2,3-d][1,3]dioxol-5-yl)pyrimidin-4-amine and 6-(2-chloro-6-(trifluoromethyl)pyridin-3-yl)-N-((3aS,9aR)-2,2-dimethyl-3a,4,9,9a-tetrahydronaphtho[2,3-d][1,3]dioxol-5-yl)pyrimidin-4-amine.

N-((3aR,9aS)-2,2-Dimethyl-3a,4,9,9a-tetrahydronaphtho[2,3-d][1,3]dioxol-5-yl)-6-iodopyrimidin-4-amine and N-((3aS,9aR)-2,2-dimethyl-3a,4,9,9a-tetrahydronaphtho[2,3-d][1,3]dioxol-5-yl)-6-iodopyrimidin-4-amine from step (a) above were reacted with 2-chloro-3-(tributylstannyl)-6-(trifluoromethyl)pyridine (Example 5(b)) under the conditions of Example 5(c) to provide the title compound as an amber resin. MS (ESI, pos. ion) m/z: 477 [M+1]. ¹H NMR (400 MHz, chloroform-d) δ ppm: 0.90 (s, 3 H), 1.23 (s, 3 H), 2.49 (d, J=14.87 Hz, 1 H), 2.70 (dd, J=14.97, 2.84 Hz, 1 H), 2.93-3.00 (m, 1 H), 3.10 (dd, J=2.25, 15.36 Hz, 1 H), 4.59-4.68 (m, 2 H), 6.74 (s, 1 H), 7.18-7.26 (m, 3 H), 7.69 (d, J=7.82 Hz, 1 H), 8.14 (d, J=7.63 Hz, 1 H), 8.81 (s, 1 H). 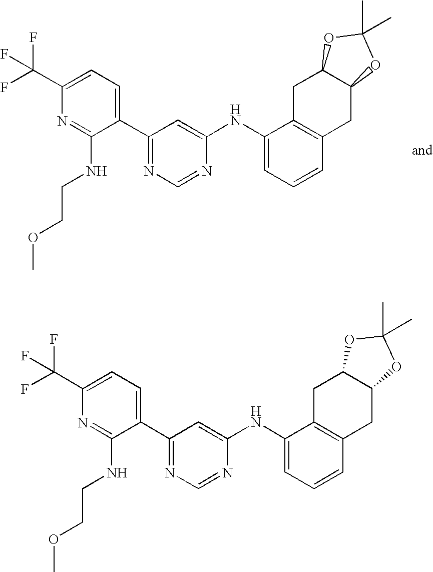
(c) N-((3aR,9aS)-2,2-Dimethyl-3a,4,9,9a-tetrahydronaphtho[2,3-d][1,3]dioxol-5-yl)-6-(2-(2-methoxyethylamino)-6-(trifluoromethyl)pyridin-3-yl)pyrimidin-4-amine and N-((3aS,9aR)-2,2-Dimethyl-3a,4,9,9a-tetrahydronaphtho[2,3-d][1,3]dioxol-5-yl)-6-(2-(2-methoxyethylamino)-6-(trifluoromethyl)pyridin-3-yl)pyrimidin-4-amine.

A glass microwave reaction vessel was charged with 6-(2-chloro-6-(trifluoromethyl)pyridin-3-yl)-N-((3aR,9aS)-2,2-dimethyl-3a,4,9,9a-tetrahydronaphtho[2,3-d][1,3]dioxol-5-yl)pyrimidin-4-amine and 6-(2-chloro-6-(trifluoromethyl)pyridin-3-yl)-N-((3aS,9aR)-2,2-dimethyl-3a,4,9,9a-tetrahydronaphtho[2,3-d][1,3]dioxol-5-yl)pyrimidin-4-amine from step (b) above (0.320 g, 0.671 mmol), 2-methoxyethylamine (0.10 g, 1.3 mmol, Aldrich), and NMP (2.5 mL). The reaction mixture was magnetically stirred and heated in a Discover® model microwave reactor (CEM, Matthews, N.C.) at 120° C. for 30 min. An additional amount of 2-methoxyethylamine (0.10 g, 1.34 mmol) was added and heating repeated at 120° C. for 30 min. An additional amount of 2-methoxy-ethylamine (0.10 g, 1.34 mmol) was added and heating repeated at 120° C. for 10 min. The reaction mixture was diluted with EtOAc (100 mL) and washed with 10% Na₂CO₃ (75 mL) and water (2×50 mL). The aqueous phases were combined and back-extracted with EtOAc (2×50 mL). The organic extracts were combined and washed with satd NaCl (50 mL), dried over Na₂SO₄, filtered and concentrated in vacuo to afford 440 mg as a brown oily residue. The residue was adsorbed onto a plug of silica gel and chromatographed through a Redi-Sep® pre-packed silica gel column (40 g), eluting with a gradient of 40% to 60% EtOAc in hexane, to provide the title product as a yellow solid. MS (ESI, pos. ion) m/z: 516 [M+1]. 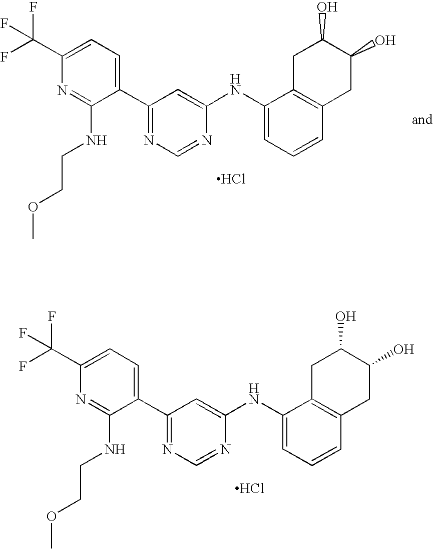
(d) (2S,3R)-5-(6-(2-(2-Methoxyethylamino)-6-(trifluoromethyl)pyridin-3-yl)pyrimidin-4-ylamino)-1,2,3,4-tetrahydronaphthalene-2,3-diol and (2R,3S)-5-(6-(2-(2-Methoxyethylamino)-6-(trifluoromethyl)pyridin-3-yl)pyrimidin-4-ylamino)-1,2,3,4-tetrahydronaphthalene-2,3-diol, hydrochloride salts.

N-((3aR,9aS)-2,2-Dimethyl-3a,4,9,9a-tetrahydro-naphtho[2,3-d][1,3]dioxol-5-yl)-6-(2-(2-methoxyethylamino)-6-(trifluoromethyl)-pyridin-3-yl)pyrimidin-4-amine and N-((3aS,9aR)-2,2-dimethyl-3a,4,9,9a-tetrahydronaphtho[2,3-d][1,3]dioxol-5-yl)-6-(2-(2-methoxyethylamino)-6-(trifluoromethyl)pyridin-3-yl)pyrimidin-4-amine from step (c) above (0.146 g, 0.28 mmol), was treated with 4 N HCl in dioxane (5 mL, Aldrich) and MeOH (5 mL). The bright yellow reaction mixture was stirred at 25° C. for 2 h, then treated with an additional aliquot of 4 N HCl in dioxane (5 mL) and MeOH (5 mL) and stirred for 5 h. The mixture was concentrated in vacuo, diluted with MeOH (50 mL) and treated with concd HCl (˜50 μL), then stirred at 25° C. for 1 h. The reaction mixture was concentrated in vacuo to afford the title product as an orange amorphous solid. MS (ESI, pos. ion) m/z: 476 [M+1]. Anal. Calcd for C, 53.96; H, 4.92; N, 13.68; F, 11.13; Cl, 6.93. Found C, 52.15; H, 5.30; N, 11.94; F, 10.29; Cl, 8.11.

### EXAMPLE 57

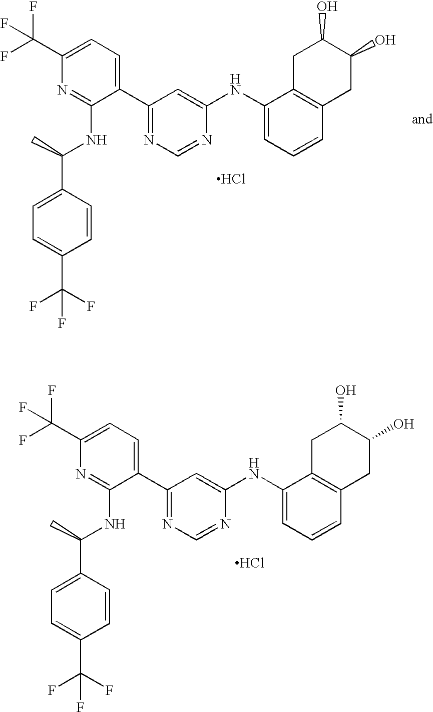

### (2S,3R)-5-(6-(6-(Trifluoromethyl)-2-((R)-1-(4-(trifluoromethyl)phenyl)ethylamino)pyridin-3-yl)pyrimidin-4-ylamino)-1,2,3,4-tetrahydro-naphthalene-2,3-diol and (2R,3S)-5-(6-(6-(1(Trifluoromethyl)-2-((R)-1-(4-(trifluoromethyl)phenyl)ethylamino)pyridin-3-yl)pyrimidin-4-ylamino)-1,2,3,4-tetrahydronaphthalene-2,3-diol, hydrochloride salts

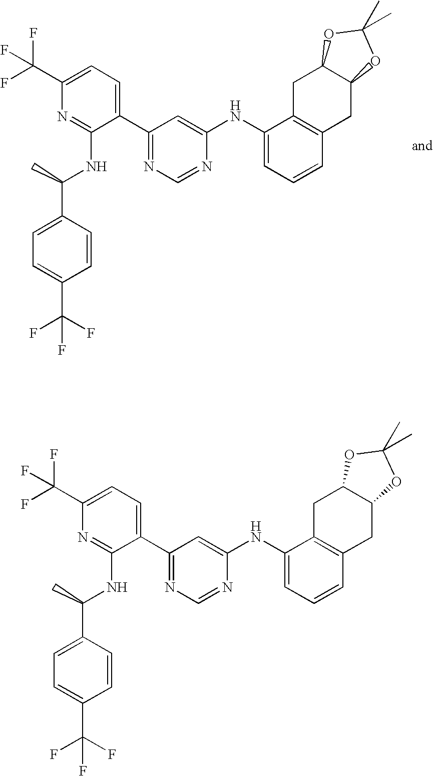 (a) N-((3aR,9aS)-2,2-Dimethyl-3a,4,9,9a-tetrahydronaphtho[2,3-d][1,3]dioxol-5-yl)-6-(6-(trifluoromethyl)-2-((R)-1-(4-(trifluoromethyl)phenyl)-ethylamino)pyridin-3-yl)pyrimidin-4-amine and N-((3aS,9aR)-2,2-Dimethyl-3a,4,9,9a-tetrahydronaphtho[2,3-d][1,3]dioxol-5-yl)-6-(6-(trifluoromethyl)-2-((R)-1-(4-(trifluoromethyl)phenyl)ethylamino)pyridin-3-yl)pyrimidin-4-amine.

According to the procedure described for Example 56(c) above, (R)-1-(4-fluorophenyl)ethanamine (0.19 g, 1.3 mmol, Synquest) and 6-(2-chloro-6-(trifluoromethyl)pyridin-3-yl)-N-((3aR,9aS)-2,2-dimethyl-3a,4,9,9a-tetrahydronaphtho[2,3-d][1,3]dioxol-5-yl)pyrimidin-4-amine and 6-(2-chloro-6-(trifluoromethyl)pyridin-3-yl)-N-((3aS,9aR)-2,2-dimethyl-3a,4,9,9a-tetrahydronaphtho[2,3-d][1,3]dioxol-5-yl)pyrimidin-4-amine (0.320 g, 0.671 mmol, Example 56(b)), provided the title product. MS (ESI, pos. ion) m/z: 580 [M+1]. 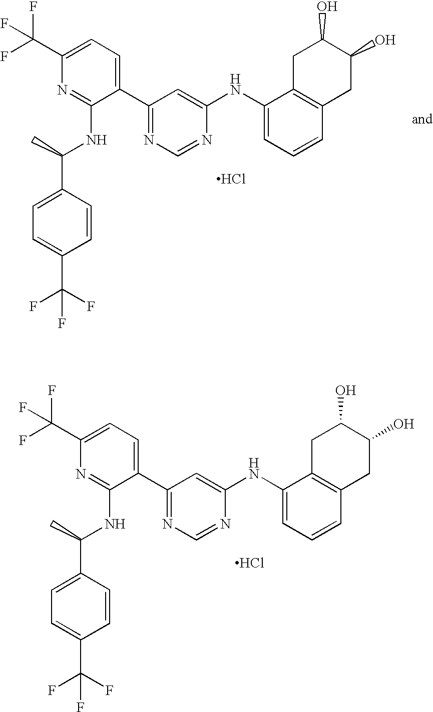
(b) (2S,3R)-5-(6-(6-(Trifluoromethyl)-2-((R)-1-(4-(trifluoromethyl)phenyl)-ethylamino)pyridin-3-yl)pyrimidin-4-ylamino)-1,2,3,4-tetrahydro-naphthalene-2,3-diol and (2R,3S)-5-(6-(6-(Trifluoromethyl)-2-((R)-1-(4-(trifluoromethyl)phenyl)ethylamino)pyridin-3-yl)pyrimidin-4-ylamino)-1,2,3,4-tetrahydronaphthalene-2,3-diol, hydrochloride salts

According to the procedure described for Example 56(d), N-((3aR,9aS)-2,2-dimethyl-3a,4,9,9a-tetrahydronaphtho[2,3-d][1,3]dioxol-5-yl)-6-(6-(trifluoromethyl)-2-((R)-1-(4-(trifluoromethyl)phenyl)ethylamino)pyridin-3-yl)pyrimidin-4-amine and N-((3aS,9aR)-2,2-dimethyl-3a,4,9,9a-tetrahydronaphtho[2,3-d][1,3]dioxol-5-yl)-6-(6-(trifluoromethyl)-2-((R)-1-(4-(trifluoromethyl)phenyl)ethylamino)pyridin-3-yl)pyrimidin-4-amine from step (a) above provided the title product as a yellow amorphous solid. MS (ESI, pos. ion) m/z: 540 [M+1]. Anal. Calcd for C, 58.39; H, 4.55; N, 12.16; F, 13.19; Cl, 6.16. Found C, 57.17; H, 4.93; N, 10.38; F, 11.60; Cl, 7.69.

### EXAMPLE 58

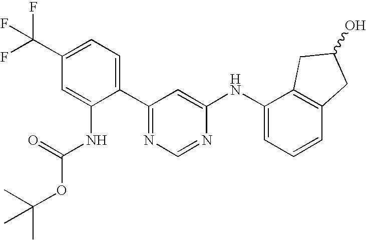

### tert-Butyl 2-(6-(2-hydroxy-2,3-dihydro-1H-inden-4-ylamino)pyrimidin-4-yl)-5-(trifluoromethyl)phenylcarbamate

4-Amino-2,3-dihydro-1H-inden-2-ol (149 mg, 999 μmol, Example 27(d)) was reacted with tert-butyl 2-(6-chloropyrimidin-4-yl)-5-(trifluoromethyl)phenylcarbamate (373 mg, 999 μmol, prepared according WO 2004/014871) under the conditions of Example 1(b)) to give the title compound as an amorphous solid. MS (ESI, pos. ion) m/z: 487 [M+1].

### EXAMPLE 59

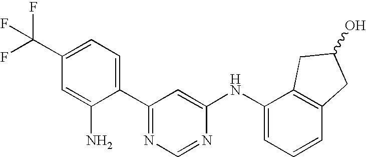

### 4-(6-(2-Amino-4-(trifluoromethyl)phenyl)pyrimidin-4-ylamino)-2,3-dihydro-1H-inden-2-ol

The title compound was formed as a side product of the reaction described in Example 58, and was isolated as an amorphous solid. MS (ESI, pos. ion) m/z: 387 [M+1].

### EXAMPLE 60

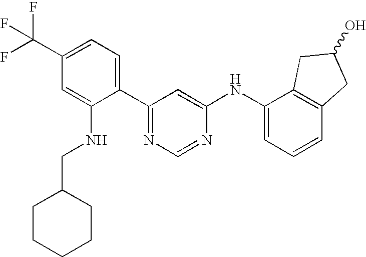

### 4-(6-(2-(Cyclohexylmethylamino)-4-(trifluoromethyl)phenyl)pyridin-4-ylamino)-2,3-dihydro-1H-inden-2-ol

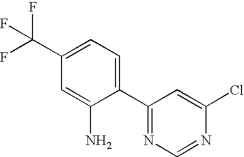 (a) 2-(6-Chloropyrimidin-4-yl)-5-(trifluoromethyl)benzenamine.

To a solution of tert-butyl 2-(6-chloropyrimidin-4-yl)-5-(trifluoromethyl)phenylcarbamate (11 g, 30 mmol, prepared according WO 2004/014871) in CH₂Cl₂ (20 mL) was added trifluoroacetic acid (20 mL, 269 mmol, Aldrich) dropwise over a period of 20 min with stirring at 0° C. The reaction mixture was stirred at room temperature for 2 h, diluted with toluene (50 mL) and evaporated under reduced pressure. The residue was dissolved in EtOAc (100 mL), washed with satd NaHCO₃ (50 mL) and brine (5 mL), dried over Na₂SO₄, and filtered. The filtrate was evaporated in vacuo and the residue purified by silica gel column chromatography (20% EtOAc/hexanes) to give the title compound as an amorphous solid. MS (ESI, pos. ion) m/z: 273 [M+1]. 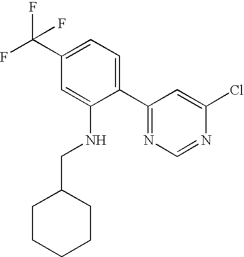
(b) 2-(6-Chloropyrimidin-4-yl)-N-(cyclohexylmethyl)-5-(trifluoromethyl)-benzenamine.

A mixture of 2-(6-chloropyrimidin-4-yl)-5-(trifluoromethyl)-benzenamine from step (a) above (1.75 g, 6.40 mmol), cyclohexanecarbaldehyde (0.85 mL, 7.04 mmol, Fluka), acetic acid (0.74 mL, 12.9 mmol) and sodium triacetoxyborohydride (1.90 g, 8.97 mmol) in 1,2-dichloroethane (20 mL) was stirred at room temperature for 1.5 h. The reaction mixture was diluted with water (50 mL), the layers were separated, and the aqueous phase was extracted with EtOAc (2×50 mL). The combined organic phases were washed with satd aqueous NaHCO₃ (100 mL) and brine (100 mL), dried over sodium sulfate, and filtered. The filtrate was evaporated in vacuo and the residue purified by silica gel chromatography (10% EtOAc/hexanes) to give the title compound as an orange solid. MS (ESI, pos. ion) m/z: 370 [M+1]. 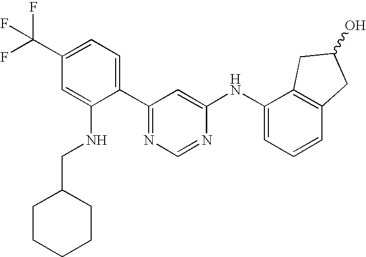
(c) 4-(6-(2-(Cyclohexylmethylamino)-4-(trifluoromethyl)phenyl)pyrimidin-4-ylamino)-2,3-dihydro-1H-inden-2-ol.

To a 50-mL, round-bottomed flask was added toluene (5 mL), 2-(6-chloropyrimidin-4-yl)-N-(cyclohexylmethyl)-5-(trifluoromethyl)benzenamine from step (b) above (112 mg, 302 μmol), 4-amino-2,3-dihydro-1H-inden-2-ol (45 mg, 302 μmol, Example 27(d)), acetao(2′-di-t-butylphosphino-1,1′-biphenyl-2-yl)palladium (II) (14 mg, 30 μmol, Sterm), and sodium tert-butoxide (58 mg, 603 μmol, Aldrich). The reaction mixture was heated at 95° C. with stirring for 2 h. The mixture was left to reach room temperature, diluted with satd NH₄Cl (20 mL) and extracted with EtOAc (2×30 mL). The combined organic extracts were washed with water (10 mL) and brine NaCl (10 mL), dried over Na₂SO₄, and filtered. The filtrate was evaporated in vacuo, and the residue was purified by silica gel chromatography (60% EtOAc/hexanes) to give the title compound as an amorphous solid. MS (ESI, pos. ion) m/z: 483 [M+1].

### EXAMPLE 61

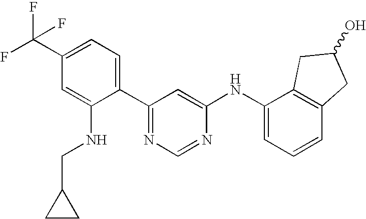

### 4-(6-(2-(Cyclopropylmethylamino)-4-(trifluoromethyl)phenyl)pyrimidin-4-ylamino)-2,3-dihydro-1H-inden-2-ol

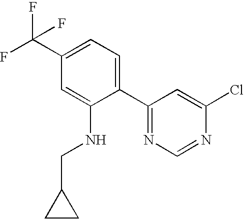 (a) 2-(6-Chloropyrimidin-4-yl)-N-(cyclopropylmethyl)-5-(trifluoromethyl)benzenamine.

2-(6-Chloropyrimidin-4-yl)-5-(trifluoromethyl)-benzenamine (273 mg, 998 μmol, Example 60(a)) was reacted with cyclopropanecarboxaldehyde (69.9 mg, 998 μmol, Aldrich) under the conditions of Example 60(b) to give the title compound as an amorphous solid. MS (ESI, pos. ion) m/z: 328 [M+1]. 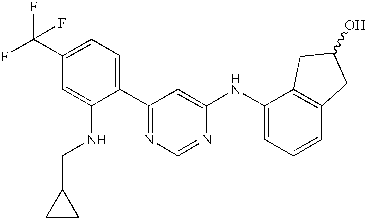
(b) 4-(6-(2-(Cyclopropylmethylamino)-4-(trifluoromethyl)phenyl)-pyrimidin-4-ylamino)-2,3-dihydro-1H-inden-2-ol.

2-(6-Chloropyrimidin-4-yl)-N-(cyclopropylmethyl)-5-(trifluoromethyl)benzenamine from step (a) above (65 mg, 198 μmol) was reacted with 4-amino-2,3-dihydro-1H-inden-2-ol (30 mg, 198 μmol, Example 27(d)) under the conditions of Example 60(c) to give the title compound as an amorphous solid. MS (ESI, pos. ion) m/z: 441 [M+1].

### EXAMPLE 62

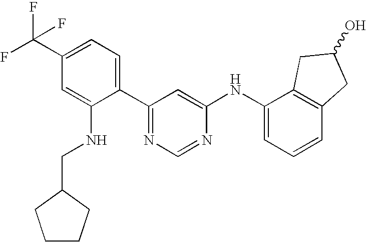

### 4-(6-(2-(Cyclopentylmethylamino)-4-(trifluoromethyl)phenyl)pyrimidin-4-ylamino)-2,3-dihydro-1H-inden-2-ol

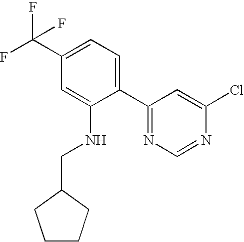 (a) 2-(6-Chloropyrimidin-4-yl)-N-(cyclopentylmethyl)-5-(trifluoromethyl)benzenamine.

2-(6-Chloropyrimidin-4-yl)-5-(trifluoromethyl)-benzenamine (273 mg, 998 μmol, Example 60(a)) was reacted with cyclopentanecarboxaldehyde (108 μl, 1097 μmol, Aldrich), under the conditions of Example 60(b) to give the title compound as an amorphous solid. MS (ESI, pos. ion) m/z: 356 [M+1]. 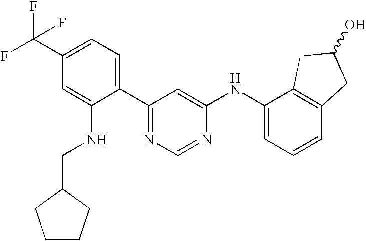
(b) 4-(6-(2-(Cyclopentylmethylamino)-4-(trifluoromethyl)phenyl)-pyrimidin-4-ylamino)-2,3-dihydro-1H-inden-2-ol.

2-(6-Chloropyrimidin-4-yl)-N-(cyclopentylmethyl)-5-(trifluoromethyl)benzenamine from step (a) above (68 mg, 191 μmol) was reacted with 4-amino-2,3-dihydro-1H-inden-2-ol (30 mg, 198 μmol, Example 27(d)) under the conditions of Example 60(c) to give the title compound as an amorphous solid. MS (ESI, pos. ion) m/z: 469 [M+1].

### EXAMPLE 63

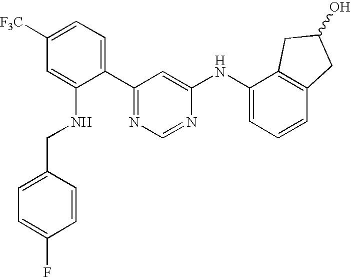

### 4-(6-(2-(4-(Fluorobenzylamino)-4-(trifluoromethyl)phenyl)pyrimidin-4-ylamino)-2,3-dihydro-1H-inden-2-ol

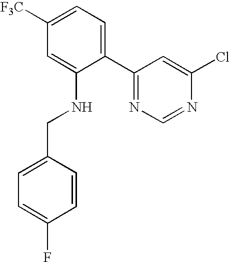 (a) N-(4-Fluorobenzyl)-2-(6-chloropyrimidin-4-yl)-5-(trifluoromethyl)benzenamine.

2-(6-Chloropyrimidin-4-yl)-5-(trifluoromethyl)-benzenamine ((3.55 g, 13.0 mmol, Example 60(a)) was reacted with 4-fluorobenzaldehyde (1.70 mL, 15.9 mmol, Aldrich) under the conditions of Example 60(b) to give the title compound as an amorphous solid. MS (ESI, pos. ion) m/z: 382 [M+1]. 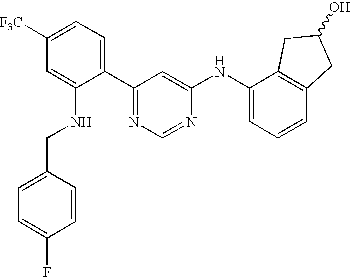
(b) 4-(6-(2-(4-Fluorobenzylamino)-4-(trifluoromethyl)phenyl)pyrimidin-4-ylamino)-2,3-dihydro-1H-inden-2-ol.

A mixture of N-(4-fluorobenzyl)-2-(6-chloropyrimidin-4-yl)-5-(trifluoromethyl)benzenamine from step (a) above (0.276 g, 0.722 mmol) and 4-amino-2,3-dihydro-1H-inden-2-ol (0.115 g, 0.772 mmol, Example 27(d)) in EtOH (2 mL) was heated in a microwave reactor at 160° C. for 20 min. The reaction mixture was left to reach room temperature and evaporated under reduced pressure. The residue was partitioned between satd aqueous NaHCO₃ (30 mL) and EtOAc (30 mL). The organic phase was separated, washed with brine (30 mL), dried over sodium sulfate, and filtered. The filtrate was evaporated in vacuo and the residue purified by silica gel column chromatography (gradient: 30-80% EtOAc in hexanes) to afford the title compound as yellow solid. MS (ESI, pos. ion) m/z: 495 [M+1].

### EXAMPLE 64

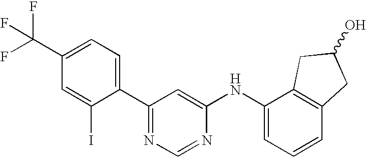

### 4-(6-(2-Iodo-4-(trifluoromethyl)phenyl)pyrimidin-4-ylamino)-2,3-dihydro-1H-inden-2-ol

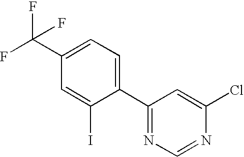 (a) 4-Chloro-6-(2-iodo-4-(trifluoromethyl)phenyl)pyrimidine.

To a 100-mL, round-bottomed flask was added iodine (3.81 g, 15 mmol, Aldrich), 1,1,1,2,2,2-hexamethyldisilane (2 g, 15 mmol, Aldrich), benzyltriethylammonium chloride (0.11 g, 0.5 mmol, Aldrich) and 2-(6-chloropyrimidin-4-yl)-5-(trifluoromethyl)benzenamine (1.36 g, 5 mmol, Example 60(a)) in CCl₄ (100 mL) at 0° C. The reaction mixture was stirred for 56 h at room temperature, diluted with water (30 mL), and extracted with EtOAc (2×40 mL). The combined organic extract was washed with water (20 mL), 1N Na₂S₂O₃ (30 ml), and satd NaCl (20 mL), dried over Na₂SO₄, and filtered. The filtrate was evaporated in vacuo and the residue purified by silica gel column chromatography (20% EtOAc/hexanes) to give the title compound as amorphous solid. MS (ESI, pos. ion) m/z: 384 [M+1]. 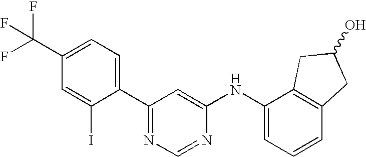
(b) 4-(6-(2-Iodo-4-(trifluoromethyl)phenyl)pyrimidin-4-ylamino)-2,3-dihydro-1H-inden-2-ol.

A mixture of 4-amino-2,3-dihydro-1H-inden-2-ol (74.5 mg, 499 μmol, Example 27(d)), 4-chloro-6-(2-iodo-4-(trifluoromethyl)phenyl)-pyrimidine from step (a) above (192 mg, 499 μmol), and diisopropylethylamine (104 μl, 599 μmol, Aldrich) in EtOH (0.5 mL) was heated in a Smith Synthesizer™ microwave reactor at 170° C. for 30 min. The reaction mixture was diluted with water (10 mL) and extracted with EtOAc (2×20 mL). The combined organic extract was washed with water (10 mL), satd NaCl (10 mL), dried over Na₂SO₄, and filtered. The filtrate was evaporated in vacuo and the residue purified by silica gel column chromatography (EtOAc) to give the title compound as amorphous solid. MS (ESI, pos. ion) m/z: 498 [M+1].

### EXAMPLE 65

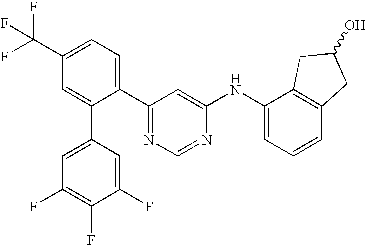

### 4-(6-(2-(3,4,5-Trifluorophenyl)-4-(trifluoromethyl)phenyl)pyrimidin-4-ylamino)-2,3-dihydro-1H-inden-2-ol

A mixture of 4-(6-(2-iodo-4-(trifluoromethyl)phenyl)pyrimidin-4-ylamino)-2,3-dihydro-1H-inden-2-ol (50 mg, 101 μmol, Example 64(b)), 3,4,5-trifluorobenzeneboronic acid (27 mg, 151 μmol, Aldrich), tetrakis(triphenylphosphine)palladium(0) (12 mg, 10 μmol, Aldrich), and sodium carbonate (201 μmol) in dioxane (1 mL), was heated in a Smith Synthesizer™ microwave reactor at 160° C. for 30 min. The reaction mixture was left to reach room temperature, and was diluted with water (10 mL), and extracted with EtOAc (2×20 mL). The combined organic extract was washed with water (10 mL), satd NaCl (10 mL), dried over Na₂SO₄, and filtered. The filtrate was evaporated in vacuo and the residue purified by silica gel column chromatography (EtOAc) to give the title compound as amorphous solid. MS (ESI, pos. ion) m/z: 502 [M+1].

### EXAMPLE 66

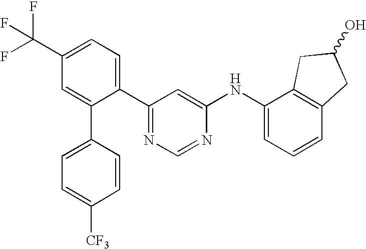

### 4-(6-(2-(4-Trifluoromethylphenyl)-4-(trifluoromethyl)phenyl)pyrimidin-4-ylamino)-2,3-dihydro-1H-inden-2-ol

4-(6-(2-Iodo-4-(trifluoromethyl)phenyl)-pyrimidin-4-ylamino)-2,3-dihydro-1H-inden-2-ol (50 mg, 101 μmol, Example 64(b)) was reacted with 4-(trifluoromethyl)phenylboronic acid (38 mg, 201 μmol, Aldrich) under the conditions of Example 65 to give the title compound as an amorphous solid. MS (ESI, pos. ion) m/z: 516 [M+1]

### EXAMPLE 67

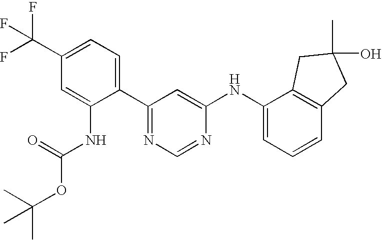

### tert-Butyl 2-(6-(2-hydroxy-2-methyl-2,3-dihydro-1H-inden-4-ylamino)-pyrimidin-4-yl)-5-(trifluoromethyl)phenylcarbamate

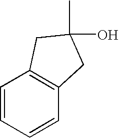 (a) 2-Methyl-2,3-dihydro-1H-inden-2-ol.

To a 250-mL, round-bottomed flask was added cerium(III) chloride heptahydrate (56 g, 150 mmol, Aldrich) and dried by heating at 140° C. under vacuum (oil pump) for 2 h. After cooling down to 0° C., THF (100 mL) was added and the mixture was then treated in a ultrasonic bath for 18 h. The mixture was cooled down to −78° C., methyllithium (48 mL, 120 mmol) was added dropwise, and the mixture was stirred at −78° C. for 2 h. A solution of 2-indanone (13.2 g, 100 mmol, Aldrich) in THF (20 mL) was then added dropwise and the mixture was stirred −78° C. for 2 h. The reaction mixture was left to reach room temperature, diluted with satd NH₄Cl (100 mL), and extracted with EtOAc (2×150 mL). The combined organic extract was washed with water (10 mL), satd NaCl (10 mL), dried over Na₂SO₄, and filtered. The filtrate was evaporated in vacuo and the residue purified by silica gel column chromatography (30% EtOAc/hexanes) to give the title compound as amorphous solid. MS (ESI, pos. ion) m/z: 149 [M+1]. 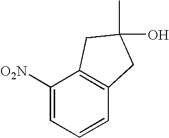
(b) 2-Methyl-4-nitro-2,3-dihydro-1H-inden-2-ol.

A solution of 2-methyl-2,3-dihydro-1H-inden-2-ol from step (a) above (2.96 g, 19.97 mmol) in CH₂Cl₂ (3 mL) was added dropwise to a mixture of 36 N sulfuric acid (1.69 mL, 19.97 mmol) and nitric acid (red fuming, 0.84 mL, 19.97 mmol, Aldrich) in CH₂Cl₂ (50 mL) with stirring at 0° C. After the addition, the mixture was stirred at 0° C. for 2 min and treated with crushed ice (50 g). The reaction mixture was extracted with EtOAc (2×100 mL). The combined organic extract was washed with water (100 mL), satd NaHCO₃ (80 mL), satd NaCl (80 mL), dried over Na₂SO₄, and filtered. The filtrate was evaporated in vacuo and the residue purified by silica gel column chromatography (40% EtOAc/hexanes) to give the title compound as amorphous solid. MS (ESI, pos. ion) m/z: 194 [M+1]. 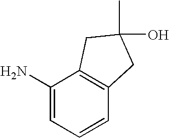
(c) 4-Amino-2-methyl-2,3-dihydro-1H-inden-2-ol.

A solution of 2-methyl-4-nitro-2,3-dihydro-1H-inden-2-ol from step (b) above (82 mg, 424 μmol) in MeOH (2 mL) was flushed with nitrogen gas and 10% Pd/C (2.3 mg, 21 μmol, Aldrich) was added. The mixture was then stirred under hydrogen atmosphere for 4 h. The catalyst was filtered through a pad of Celite®, and the filter cake was washed with EtOAc. The combined filtrates were evaporated in vacuo, and the residue purified by silica gel column chromatography (80% EtOAc/hexanes) to give the title compound as amorphous solid. MS (ESI, pos. ion) m/z: 164 [M+1]. 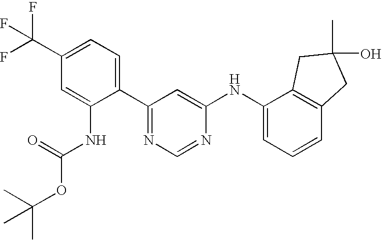
(d) tert-Butyl 2-(6-(2-hydroxy-2-methyl-2,3-dihydro-1H-inden-4-ylamino)pyrimidin-4-yl)-5-(trifluoromethyl)phenylcarbamate.

4-Amino-2-methyl-2,3-dihydro-1H-inden-2-ol from step (c) above (50 mg, 306 μmol) was reacted with tert-butyl 2-(6-chloropyrimidin-4-yl)-5-(trifluoromethyl)-phenylcarbamate (114 mg, 306 μmol, prepared according WO 2004/014871) under the conditions of Example 1(b) to give the title compound as amorphous solid. MS (ESI, pos. ion) m/z: 501 [M+1].

### EXAMPLE 68

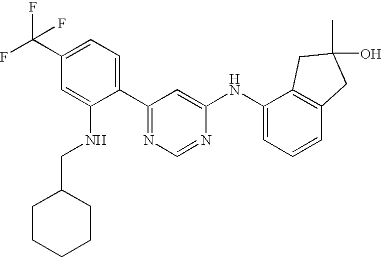

### 4-(6-(2-(Cyclohexylmethylamino)-4-(trifluoromethyl)phenyl)pyrimidin-4-ylamino)-2-methyl-2,3-dihydro-1H-inden-2-ol

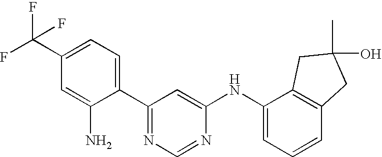 (a) 4-(6-(2-Amino-4-(trifluoromethyl)phenyl)pyrimidin-4-ylamino)-2-methyl-2,3-dihydro-1H-inden-2-ol.

4-Amino-2-methyl-2,3-dihydro-1H-inden-2-ol (50 mg, 306 μmol, Example 67(c)) was reacted with tert-butyl 2-(6-chloropyrimidin-4-yl)-5-(trifluoromethyl)phenylcarbamate (114 mg, 306 μmol, prepared according WO 2004/014871) under the conditions of Example 1(b) to give the title compound as amorphous solid. MS (ESI, pos. ion) m/z: 401 [M+1]. 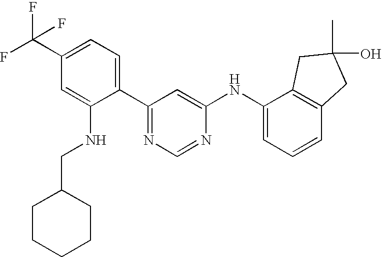
(b) 4-(6-(2-(Cyclohexylmethylamino)-4-(trifluoromethyl)phenyl)pyrimidin-4-ylamino)-2-methyl-2,3-dihydro-1H-inden-2-ol.

A glass microwave reaction vessel was charged with 4-(6-(2-amino-4-(trifluoromethyl)phenyl)pyrimidin-4-ylamino)-2-methyl-2,3-dihydro-1H-inden-2-ol from step (a) above (20 mg, 50 μmol), cyclohexanecarboxaldehyde (7 mg, 60 μmol, Aldrich), and sodium triacetoxyborohydride (16 mg, 75 μmol, Aldrich). The reaction mixture was heated in a Smith Synthesizer™ microwave reactor at 100° C. for 30 min. The solvent was removed in vacuo and the residue was purified by silica gel chromatography (60% EtOAc/hexanes) to give the title compound as amorphous solid. MS (ESI, pos. ion) m/z: 497 [M+1].

### EXAMPLE 69

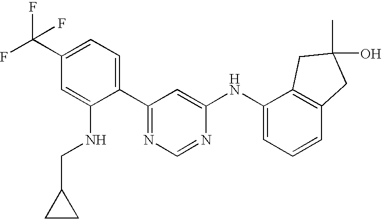

### 4-(6-(2-(Cyclopropylmethylamino)-4-(trifluoromethyl)phenyl)pyrimidin-4-ylamino)-2-methyl-2,3-dihydro-1H-inden-2-ol

4-(6-(2-Amino-4-(trifluoromethyl)phenyl)pyrimidin-4-ylamino)-2-methyl-2,3-dihydro-1H-inden-2-ol (20 mg, 50 μmol, Example 68(a)) was reacted with cyclopropanecarboxaldehyde (4.2 mg, 60 μmol, Aldrich) under the conditions of Example 68(b)) to give the title compound as amorphous solid. MS (ESI, pos. ion) m/z: 455 [M+1].

### EXAMPLE 70

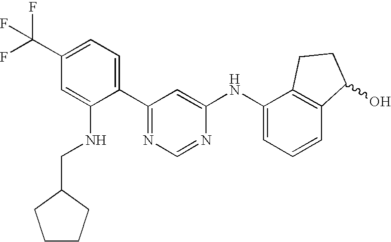

### 4-(6-(2-(Cyclopentylmethylamino)-4-(trifluoromethyl)phenyl)pyrimidin-4-ylamino)-2,3-dihydro-1H-inden-1-ol

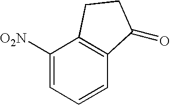 (a) 4-Nitro-2,3-dihydroinden-1-one.

A mixture of 4-nitroindan (2.45 g, 15 mmol, Aldrich) and chromium(III) oxide (4.56 g, 30 mmol, Aldrich) in acetic acid (50 mL) was stirred for 18 h at room temperature. The reaction mixture was diluted with water (200 mL) and extracted with EtOAc (2×100 mL). The combined organic extract was washed with water (100 mL), satd NaHCO₃ (80 mL), satd NaCl (80 mL), dried over Na₂SO₄, and filtered. The filtrate was evaporated in vacuo and the residue purified by silica gel column chromatography (40% EtOAc/hexanes) to give the title compound as amorphous solid. MS (ESI, pos. ion) m/z: 178 [M+1]. 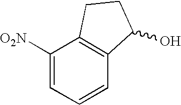
(b) 4-Nitro-2,3-dihydro-1H-inden-1-ol.

To a solution of 4-nitro-2,3-dihydroinden-1-one from step (a) above (0.70 g, 3.94 mmol) in MeOH (30 mL) was added sodium borohydride (0.165 g, 4.33 mmol, Aldrich) in small portions. After the addition, the mixture was stirred at room temperature for 10 min. The solvent was removed in vacuo, the residue was diluted with water (20 mL) and extracted with EtOAc (2×30 mL). The combined organic extract was washed with water (100 mL), satd NaHCO₃ (80 mL), satd NaCl (80 mL), dried over Na₂SO₄, and filtered. The filtrate was evaporated in vacuo and the residue purified by silica gel column chromatography (40% EtOAc/hexanes) to give the title compound as amorphous solid. MS (ESI, pos. ion) m/z: 180 [M+1]. 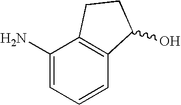
(c) 4-Amino-2,3-dihydro-1H-inden-1-ol.

Hydrogenation of 4-nitro-2,3-dihydro-1H-inden-1-ol from step (b) above (537 mg, 3.0 mmol) under the conditions of Example 67(c) provided the title compound as an amorphous solid. MS (ESI, pos. ion) m/z: 150 [M+1]. 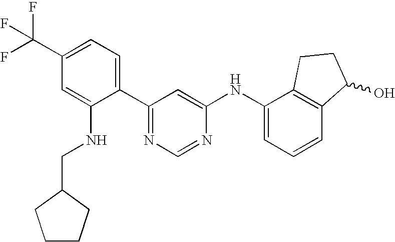
(d) 4-(6-(2-(Cyclopentylmethylamino)-4-(trifluoromethyl)phenyl)pyrimidin-4-ylamino)-2,3-dihydro-1H-inden-1-ol.

4-Amino-2,3-dihydro-1H-inden-1-ol from step (c) above (30 mg, 0.201 mmol) was reacted with 2-(6-chloropyrimidin-4-yl)-N-(cyclopentylmethyl)-5-(trifluoromethyl)benzenamine (72 mg, 0.201 mmol, Example 62(a)) under the conditions of Example 1(b) to give the title compound as amorphous solid. MS (ESI, pos. ion) m/z: 469 [M+1].

### EXAMPLE 71 AND EXAMPLE 72

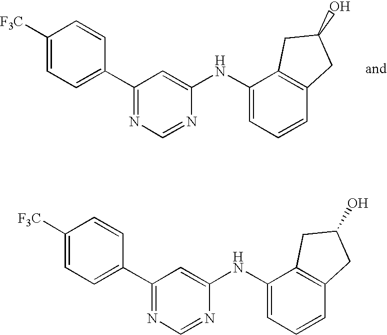

### (2S)-4-(6-(4-(Trifluoromethyl)phenyl)pyrimidin-4-ylamino)-2,3-dihydro-1H-inden-2-ol and (2R)-4-(6-(4-(Trifluoromethyl)phenyl)pyrimidin-4-ylamino)-2,3-dihydro-1H-inden-2-ol

Chiral separation (Chiralpak AD-H column; mobile phase: A: liquid CO₂, B: MeOH, 50:50 (A:B); flow rate: 60 mL/min) of the racemic mixture 4-(6-(4-(trifluoromethyl)phenyl)pyrimidin-4-ylamino)-2,3-dihydro-1H-inden-2-ol (Example 27(e)) afforded the title compounds as yellow amorphous solids. MS (ESI, pos. ion.) m/z: 372 [M+1]. The configuration (S) and (R) was assigned to each separated enantiomer at random.

### EXAMPLE 73 AND EXAMPLE 74

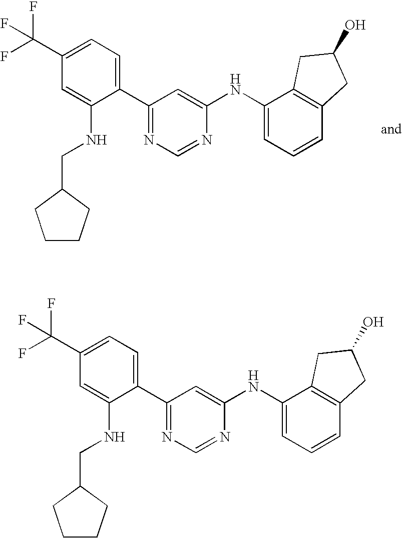

### (2S)-4-(6-(2-(Cyclopentylmethylamino)-4-(trifluoromethyl)phenyl)pyrimidin-4-ylamino)-2,3-dihydro-1H-inden-2-ol and (2R)-4-(6-(2-(Cyclopentyl-methylamino)-4-(trifluoromethyl)phenyl)pyrimidin-4-ylamino)-2,3-dihydro-1H-inden-2-ol

Chiral separation of the racemic mixture 4-(6-(2-(cyclopentyl-methylamino)-4-(trifluoromethyl)phenyl)pyrimidin-4-ylamino)-2,3-dihydro-1H-inden-2-ol (Example 62(b)) under the conditions of Examples 71 and Example 72 afforded the title compounds as amorphous solids. MS (ESI, pos. ion) m/z: 469 [M+1]. The configuration (2S) and (2R) was assigned to each separated enantiomer at random.

### EXAMPLE 75 AND EXAMPLE 76

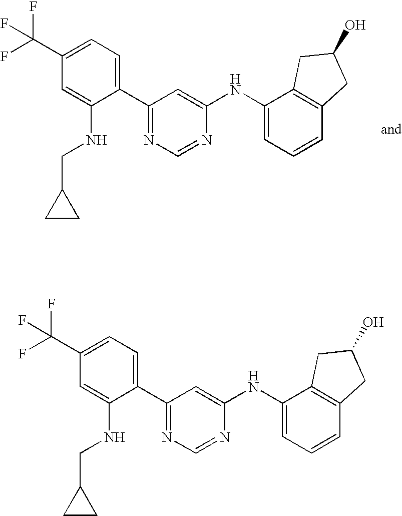

### (2S)-4-(6-(2-(Cyclopropylmethylamino)-4-(trifluoromethyl)phenyl)pyrimidin-4-ylamino)-2,3-dihydro-1H-inden-2-ol and (2R)-4-(6-(2-(Cyclopropylmethylamino)-4-(trifluoromethyl)phenyl)pyrimidin-4-ylamino)-2,3-dihydro-1H-inden-2-ol

Chiral separation of the racemic mixture 4-(6-(2-(cyclopentylmethylamino)-4-(trifluoromethyl)phenyl)pyrimidin-4-ylamino)-2,3-dihydro-1H-inden-2-ol (Example 619b)) under the conditions of Examples 71 and Example 72 afforded the title compounds as amorphous solids. MS (ESI, pos. ion) m/z: 441 [M+1]. The configuration (2S) and (2R) was assigned to each separated enantiomer at random.

### EXAMPLE 77

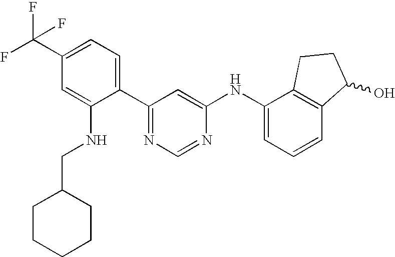

### 4-(6-(2-(Cyclohexylmethylamino)-4-(trifluoromethyl)phenyl)pyrimidin-4-ylamino)-2,3-dihydro-1H-inden-1-ol

4-Amino-2,3-dihydro-1H-inden-1-ol (30 mg, 201 μmol, Example 70(c)) was reacted with 2-(6-chloropyrimidin-4-yl)-N-(cyclohexylmethyl)-5-(trifluoromethyl)benzenamine (74 mg, 201 μmol, Example 60(b) under the conditions of Example 1(b) to give the title compound as amorphous solid. MS (ESI, pos. ion) m/z: 483 [M+1].

### EXAMPLE 78

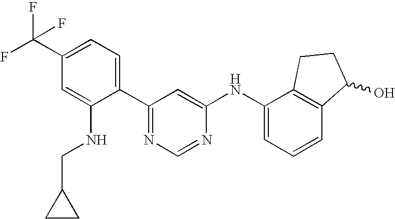

### 4-(6-(2-(Cyclopropylmethylamino)-4-(trifluoromethyl)phenyl)pyrimidin-4-ylamino)-2,3-dihydro-1H-inden-1-ol

4-Amino-2,3-dihydro-1H-inden-1-ol (30 mg, 201 μmol, Example 70(c)) was reacted with 2-(6-chloropyrimidin-4-yl)-N-(cyclopropylmethyl)-5-(trifluoromethyl)benzenamine (66 mg, 201 μmol, Example 61(a)) under the conditions of Example 1(b) to give the title compound as amorphous solid. MS (ESI, pos. ion) m/z: 441 [M+1].

### EXAMPLE 79

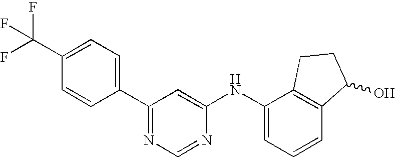

### 4-(6-(4-(1H-Trifluoromethylphenyl)pyrimidin-4-ylamino)-2,3-dihydro-1H-inden-1-ol

4-Amino-2,3-dihydro-1H-inden-1-ol (30 mg, 201 μmol, Example 70(c)) was reacted with 4-chloro-6-(4-(trifluoromethyl)phenyl)pyrimidine (52 mg, 201 μmol, Example 1(a)), under the conditions of Example 1(b) to give the title compound as amorphous solid. MS (ESI, pos. ion) m/z: 372 [M+1].

### EXAMPLE 80

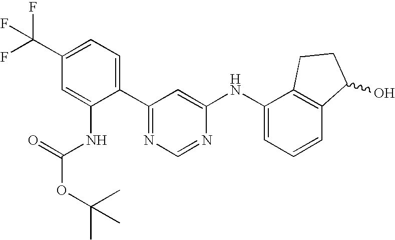

### tert-Butyl 2-(6-((1-hydroxy-2,3-dihydro-1H-inden-4-ylamino)pyrimidin-4-yl)-5-(trifluoromethyl)phenylcarbamate

4-Amino-2,3-dihydro-1H-inden-1-ol (30 mg, 201 μmol, Example 70(c)) was reacted with tert-butyl 2-(6-chloropyrimidin-4-yl)-5-(trifluoromethyl)phenylcarbamate (75 mg, 201 μmol, prepared according to WO 2004/014871) under the conditions of Example 1(b) to give the title compound as amorphous solid. MS (ESI, pos. ion) m/z: 487 [M+1].

### EXAMPLE 81

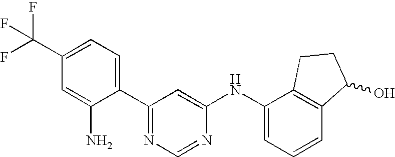

### 4-(6-(2-Amino-4-(trifluoromethyl)phenyl)pyrimidin-4-ylamino)-2,3-dihydro-1H-inden-1-ol

The title compound was formed as a side product of the reaction described in Example 80, and was isolated as an amorphous solid. MS (ESI, pos. ion) m/z: 387 [M+1].

### EXAMPLE 82

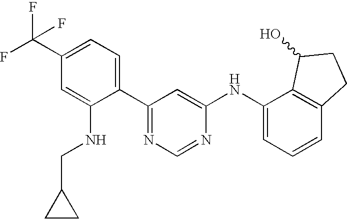

### 7-(6-(2-(Cyclopropylmethylamino)-4-(trifluoromethyl)phenyl)pyrimidin-4-ylamino)-2,3-dihydro-1H-inden-1-ol

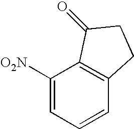 (a) 7-Nitro-2,3-dihydroinden-1-one.

4-Nitroindan (2.448 g, 15 mmol, Aldrich) was reacted with chromium(III) oxide (4.56 g, 30 mmol, Aldrich) under the conditions of Example 70(a) to give the title compound as an amorphous solid. MS (ESI, pos. ion) m/z: 178 [M+1]. 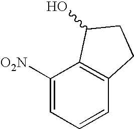
(b) 7-Nitro-2,3-dihydro-1H-inden-1-ol.

7-Nitro-2,3-dihydroinden-1-one from step (a) above (0.264 g, 1.49 mmol) was treated with sodium borohydride (0.062 g, 1.64 mmol, Aldrich) under the conditions of Example 70(b)) to give the title compound as an amorphous solid. MS (ESI, pos. ion) m/z: 180 [M+1]. 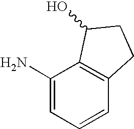
(c) 7-Amino-2,3-dihydro-1H-inden-1-ol.

7-Nitro-2,3-dihydro-1H-inden-1-ol from step (b) above (212 mg) was hydrogenated under the conditions of Example 70(c) to give the title compound as an amorphous solid. MS (ESI, pos. ion) m/z: 150 [M+1]. 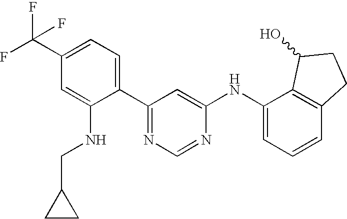
(d) 7-(6-(2-(Cyclopropylmethylamino)-4-(trifluoromethyl)phenyl)pyrimidin-4-ylamino)-2,3-dihydro-1H-inden-1-ol.

7-Amino-2,3-dihydro-1H-inden-1-ol from step (c) above (30 mg, 201 μmol) was reacted with 2-(6-chloropyrimidin-4-yl)-N-(cyclopropylmethyl)-5-(trifluoromethyl)benzenamine (66 mg, 201 μmol, Example 61(a)) under the conditions of Example 61(b) to give the title compound as an amorphous solid. MS (ESI, pos. ion) m/z: 441 [M+1].

### EXAMPLE 83

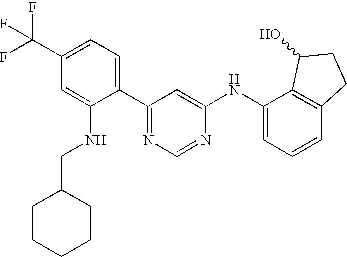

### 7-(6-(2-(Cyclohexylmethylamino)-4-(trifluoromethyl)phenyl)pyrimidin-4-ylamino)-2,3-dihydro-1H-inden-1-ol

2-(6-Chloropyrimidin-4-yl)-N-(cyclohexylmethyl)-5-(trifluoromethyl)benzenamine (74 mg, 201 μmol, Example 60(b)) was reacted with 7-amino-2,3-dihydro-1H-inden-1-ol (30 mg, 201 μmol, Example 82(c)) under the conditions of Example 1(b) to give the title compound as an amorphous solid. MS (ESI, pos. ion) m/z: 483 [M+1].

### EXAMPLE 84

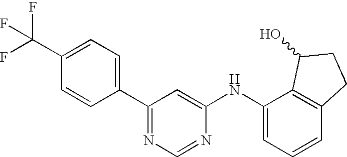

### 7-(6-(4-(Trifluoromethyl)phenyl)pyrimidin-4-ylamino)-2,3-dihydro-1H-inden-1-ol

7-Amino-2,3-dihydro-1H-inden-1-ol (30 mg, 201 μmol, Example 60(b)) was reacted with 4-chloro-6-(4-(trifluoromethyl)phenyl)pyrimidine (57 mg, 221 μmol, Example 1(a)) under the conditions of Example 61(b) to give the title compound as an amorphous solid. MS (ESI, pos. ion) m/z: 472 [M+1].

### EXAMPLE 85

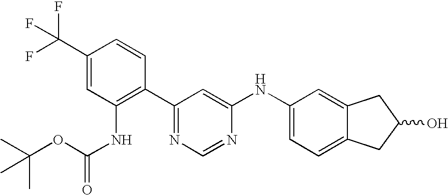

### tert-Butyl 2-(6-(2-hydroxy-2,3-dihydro-1H-inden-5-ylamino)pyrimidin-4-yl)-5-(trifluoromethyl)phenylcarbamate

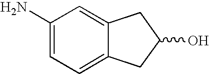 (a) 5-Amino-2,3-dihydro-1H-inden-2-ol.

5-Nitro-2,3-dihydro-1H-inden-2-ol (1 g, 6 mmol, J&W pharm Lab, LLC) was hydrogenated under the conditions of Example 70(c) to provide the title compound as off-white solid. MS (ESI, pos. ion) m/z: 150 [M+1]. 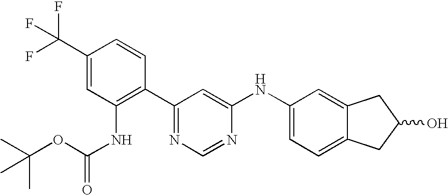
(b) tert-Butyl 2-(6-(2-hydroxy-2,3-dihydro-1H-inden-5-ylamino)pyrimidin-4-yl)-5-(trifluoromethyl)phenylcarbamate.

tert-Butyl 2-(6-chloropyrimidin-4-yl)-5-(trifluoromethyl)phenyl-carbamate (0.5 g, 1.5 mmol, prepared according to WO 2004/014871) was reacted with 5-amino-2,3-dihydro-1H-inden-2-ol from step (a) above (0.25 g, 1.7 mmol) under the conditions of Example 1(b) to provide the title compound as an off-white solid. MS (ESI, pos. ion) m/z: 487 [M+1].

### EXAMPLE 86

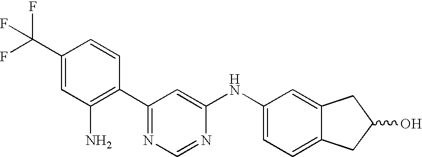

### 5-(6-(2-Amino-4-(trifluoromethyl)phenyl)pyrimidin-4-ylamino)-2,3-dihydro-1H-inden-2-ol

tert-Butyl 2-(6-(2-hydroxy-2,3-dihydro-1H-inden-5-ylamino)-pyrimidin-4-yl)-5-(trifluoromethyl)phenylcarbamate (300 mg, 617 μmol, Example 85(b)) was reacted with trifluoroacetic acid under the conditions of Example 60(a) to give the title compound as a yellow oil. MS (ESI, pos. ion) m/z: 387 [M+1].

### EXAMPLE 87

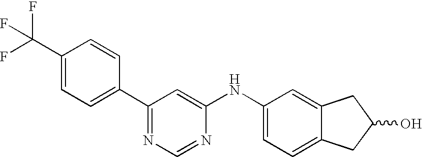

### 5-(6-(4-(Trifluoromethyl)phenyl)pyrimidin-4-ylamino)-2,3-dihydro-1H-inden-2-ol

4-Chloro-6-[4-(trifluoromethyl)phenyl]pyrimidine (180 mg, 0.7 mmol, Example 1(a)) was reacted with 5-amino-2,3-dihydro-1H-inden-2-ol (156 mg, 1.0 mmol, Example 85(a)) under the conditions of Example 1((b) to give the title compound as white amorphous solid. MS (ESI, pos. ion) m/z: 372 [M+1].

### EXAMPLE 88

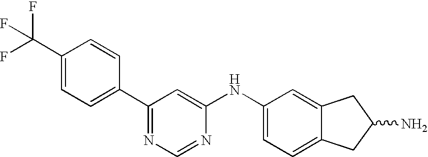

### N⁵-(6-(4-(Trifluoromethyl)phenyl)pyrimidin-4-yl)-2,3-dihydro-1H-indene-2,5-diamine

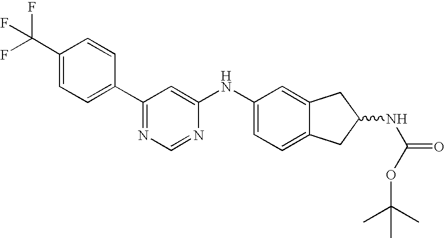 (a) tert-Butyl 5-(6-(4-(trifluoromethyl)phenyl)pyrimidin-4-ylamino)-2,3-dihydro-1H-inden-2-ylcarbamate.

A mixture of 4-chloro-6-(4-(trifluoromethyl)-phenyl)pyrimidine (0.75 g, 2.9 mmol, Example 1(a)) and tert-butyl 5-amino-2,3-dihydro-1H-inden-2-ylcarbamate (1.0 g, 4.1 mmol, J&W pharm. Lab, LLC) in NMP (10 mL) was stirred and heated at 100° C. for 1 h, and at 120° C. for 20 min in a Discover™ microwave reactor. The reaction mixture was diluted with water (20 mL) and extracted with EtOAc (5×40 mL). The combined organic extract was washed with NaHCO₃ and brine, dried over Na₂SO₄, and filtered. The filtrate was evaporated in vacuo and purified by silica gel column chromatography (gradient: 2% to 10% 2 M NH₃.MeOH in CH₂Cl₂) to provide the title compound as pale yellow solid. MS (ESI, pos. ion) m/z: 471 [M+1]. 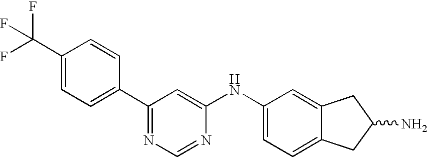
(b) N⁵-(6-(4-(Trifluoromethyl)phenyl)pyrimidin-4-yl)-2,3-dihydro-1H-indene-2,5-diamine.

tert-Butyl 5-(6-(4-(trifluoromethyl)phenyl)pyrimidin-4-ylamino)-2,3-dihydro-1H-inden-2-ylcarbamate from step (a) above (200 mg, 4.2 mmol) was treated with trifluoroacetic acid under the conditions of Example 60(a) to give the title compound as an yellow solid. MS (ESI, pos. ion) m/z: 371 [M+1].

### EXAMPLE 89

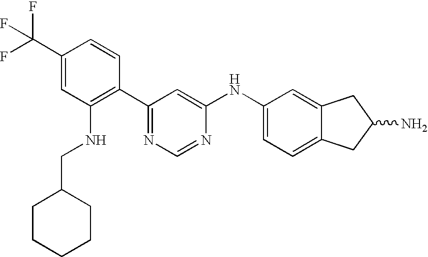

### N⁵-(6-(2-(Cyclohexylmethylamino)-4-(trifluoromethyl)phenyl)pyrimidin-4-yl)-2,3-dihydro-1H-indene-2,5-diamine

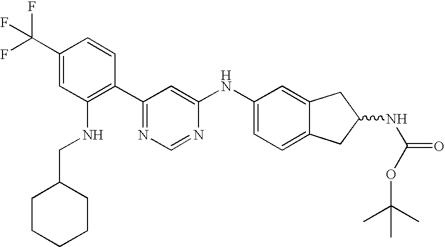 (a) tert-Butyl 5-(6-(2-(cyclohexylmethylamino)-4-(trifluoromethyl)phenyl)pyrimidin-4-ylamino)-2,3-dihydro-1H-inden-2-ylcarbamate.

2-(6-Chloropyrimidin-4-yl)-N-(cyclohexylmethyl)-5-(trifluoromethyl)benzenamine (0.16 g, 0.4 mmol, Example 60(b)) was reacted with tert-butyl 5-amino-2,3-dihydro-1H-inden-2-ylcarbamate (0.2 g, 0.9 mmol, J&W pharm Lab, LLC) under the conditions of Example 60(c) to give the title compound as a yellow amorphous solid. MS (ESI, pos. ion) m/z: 582 [M+1]. 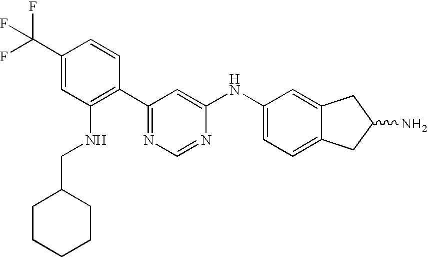
(b) N⁵-(6-(2-(Cyclohexylmethylamino)-4-(trifluoromethyl)phenyl)pyrimidin-4-yl)-2,3-dihydro-1H-indene-2,5-diamine.

A mixture of tert-butyl 5-(6-(2-(cyclohexylmethylamino)-4-(trifluoromethyl)phenyl)pyrimidin-4-ylamino)-2,3-dihydro-1H-inden-2-ylcarbamate from step (a) above (50 mg, 86 μmol) and 4 N HCl in ether (20 mL) was stirred at room temperature for 2 h. The reaction mixture was diluted with satd NaHCO₃ (20 mL) and extracted with EtOAc (3×20 mL). The combined organic extract was washed with NaHCO₃ and brine, dried over Na₂SO₄, and filtered. The filtrate was evaporated in vacuo to give the title compound as an yellow oil. MS (ESI, pos. ion) m/z: 482 [M+1].

### EXAMPLE 90

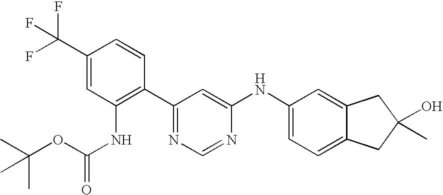

### tert-Butyl 2-(6-(2-hydroxy-2-methyl-2,3-dihydro-1H-inden-5-ylamino)-pyrimidin-4-yl)-5-(trifluoromethyl)phenylcarbamate

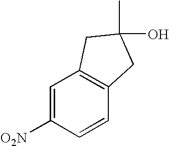 (a) 2-Methyl-5-nitro-2,3-dihydro-1H-inden-2-ol.

The title compound was formed as a second product of the nitration of 2-methyl-2,3-dihydro-1H-inden-2-ol (2.96 g, 2.0 mmol, Example 67(a)) described in Example 67(b), and was isolated as amorphous solid. MS (ESI, pos. ion) m/z: 194 [M+1]. 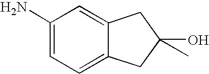
(b) 5-Amino-2-methyl-2,3-dihydro-1H-inden-2-ol.

2-Methyl-5-nitro-2,3-dihydro-1H-inden-2-ol from step (a) above (0.3 g, 2 mmol) was hydrogenated under the conditions of Example 67(c) to provide the title compound as off-white solid. MS (ESI, pos. ion) m/z: 164 [M+1]. 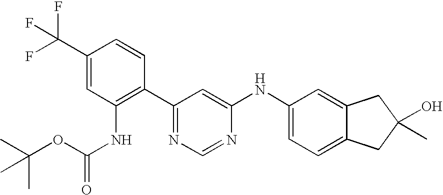
(c) tert-Butyl 2-(6-(2-hydroxy-2-methyl-2,3-dihydro-1H-inden-5-ylamino)pyrimidin-4-yl)-5-(trifluoromethyl)phenylcarbamate.

tert-Butyl 2-(6-chloropyrimidin-4-yl)-5-(trifluoromethyl)phenylcarbamate (0.2 g, 0.535 mmol, prepared according to WO 2004/014871) was reacted with 5-amino-2-methyl-2,3-dihydro-1H-inden-2-ol from step (b) above (0.131 g, 0.803 mmol) under the conditions of Example 1(b) to give the title compound as a yellow solid. MS (ESI, pos. ion) m/z: 501 [M+1].

### EXAMPLE 91

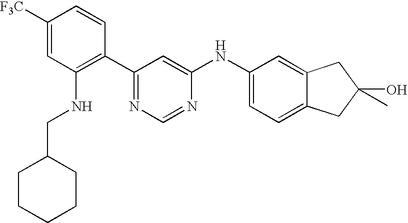

### 5-(6-(2-(Cyclohexylmethylamino)-4-(trifluoromethyl)phenyl)pyrimidin-4-ylamino)-2-methyl-2,3-dihydro-1H-inden-2-ol

2-(6-Chloropyrimidin-4-yl)-N-(cyclohexylmethyl)-5-(trifluoromethyl)benzenamine (0.1 g, 0.270 mmol, Example 60(b)) was reacted with 5-amino-2-methyl-2,3-dihydro-1H-inden-2-ol (0.066 g, 0.406 mmol, Example 90(b)) under the conditions of Example 60(c) to give the title compounds as a yellow solid. MS (ESI, pos. ion) m/z: 497 [M+1].

### EXAMPLE 92

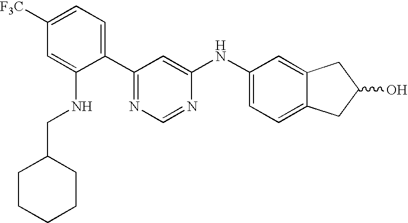

### 5-(6-(2-(Cyclohexylmethylamino)-4-(trifluoromethyl)phenyl)pyrimidin-4-ylamino)-2,3-dihydro-1H-inden-2-ol

2-(6-Chloropyrimidin-4-yl)-N-(cyclohexylmethyl)-5-(trifluoromethyl)benzenamine (0.203 g, 0.548 mmol, Example 60(b)) was reacted with 5-amino-2,3-dihydro-1H-inden-2-ol (0.087 g, 0.586 mmol, Example 85(a)) under the conditions of Example 1(b) to afford the title compound as a yellow solid. MS (ESI, pos. ion) m/z: 483 [M+1].

### EXAMPLE 93 AND EXAMPLE 94

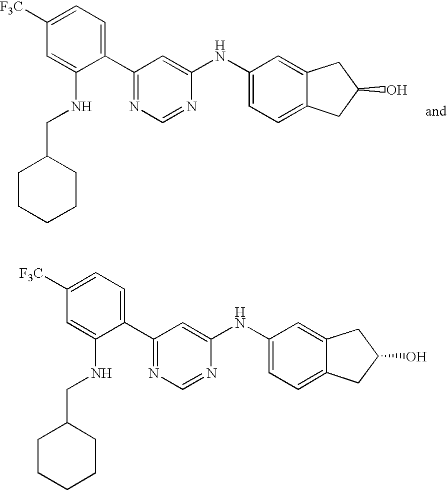

### (S)-5-(6-(2-(Cyclohexylmethylamino)-4-(trifluoromethyl)phenyl)pyrimidin-4-ylamino)-2,3-dihydro-1H-inden-2-ol and (R)-5-(6-(2-(Cyclohexylmethyl-amino)-4-(trifluoromethyl)phenyl)pyrimidin-4-ylamino)-2,3-dihydro-1H-inden-2-ol

Chiral separation of the racemic mixture 5-(6-(2-(cyclohexylmethylamino)-4-(trifluoromethyl)phenyl)pyrimidin-4-ylamino)-2,3-dihydro-1H-inden-2-ol (Example 92(d) under the conditions of Examples 71 and Example 72 afforded the title compounds as yellow amorphous solids. MS (ESI, pos. ion) m/z: 483 [M+1]. The configuration (S) and (R) was assigned to each separated enantiomer at random.

### EXAMPLE 95

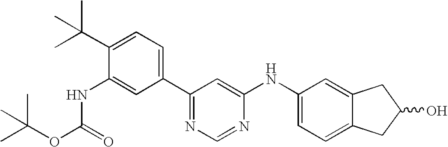

### tert-Butyl 2-tert-butyl-5-(6-(2-hydroxy-2,3-dihydro-1H-inden-5-ylamino)-pyrimidin-4-yl)phenylcarbamate

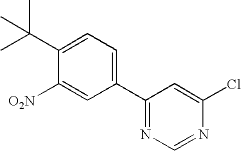 (a) 4-(4-tert-Butyl-3-nitrophenyl)-6-chloropyrimidine.

To a stirred solution of 4-(4-tert-butylphenyl)-6-chloropyrimidine (2.15 g, 8.70 mmol, prepared as described in WO 2004/014871) in concd H₂SO₄ (20 mL) was added KNO₃ (1.77 g, 17.6 mmol) in small potions at 0° C. After the addition, the reaction mixture was stirred at 0° C. for 2 h and was poured onto crushed ice. The resulting precipitate was extracted with EtOAc. The aqueous phase was neutralized by adding 6 N NaOH and solid NaHCO₃ then extracted with EtOAc. The combined EtOAc extracts were washed with water (100 mL) and brine (100 mL), dried over sodium sulfate, and filtered. The filtrate was evaporated in vacuo, and the residue was purified by silica gel column chromatography (gradient: 0-20% EtOAc in hexanes) to afford the title compound as pale-yellow crystalline solid. MS (ESI, pos. ion) m/z: 292 [M+1] 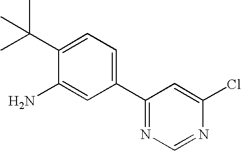
(b) 2-tert-Butyl-5-(6-chloropyrimidin-4-yl)benzenamine.

A mixture of 4-(4-tert-butyl-3-nitrophenyl)-6-chloropyrimidine from step (a) above (0.692 g, 2.37 mmol) and iron powder (0.829 g, 14.2 mmol, Aldrich) in EtOH (12.5 mL) and AcOH (7.5 mL) was refluxed for 3 h with vigorous stirring. The reaction mixture was left to reach room temperature and was filtered through a pad of Celite®. The filter cake was washed with MeOH. The filtrate was evaporated in vacuo and the residue was partitioned between 10% Na₂CO₃ (50 mL) and EtOAc (50 mL). The aqueous phase was extracted with EtOAc (2×30 mL). The combined EtOAc extracts were washed with water (2×75 mL) and brine (100 mL), dried over sodium sulfate, and filtered. The filtrate was evaporated in vacuo and the residue was purified by silica gel column chromatography (gradient: 0-30% EtOAc in hexanes) to afford the title compound as light-brown solid. MS (ESI, pos. ion) m/z: 262 [M+1]. 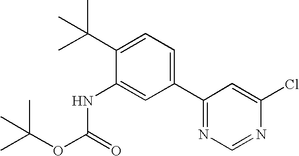
(c) tert-Butyl 2-tert-butyl-5-(6-chloropyrimidin-4-yl)phenylcarbamate.

A mixture of 2-tert-butyl-5-(6-chloropyrimidin-4-yl)benzenamine from step (b) above (0.096 g, 0.36 mmol) and di-tert-butyl dicarbonate (0.096 mL, 0.41 mmol, Aldrich) in toluene (5 mL) was refluxed for 3 days. The reaction mixture was left to reach room temperature and was evaporated in vacuo. The residue was purified by silica gel column chromatography (gradient: 0-30% EtOAc in hexanes) to afford the title compound as clear viscous oil. MS (ESI, pos. ion) m/z: 362 [M+1]. 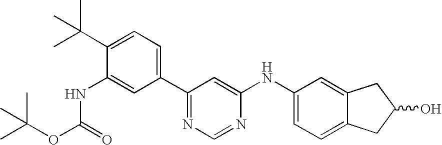
(d) tert-Butyl 2-tert-butyl-5-(6-(2-hydroxy-2,3-dihydro-1H-inden-5-ylamino)pyrimidin-4-yl)phenylcarbamate.

A mixture of tert-butyl 2-tert-butyl-5-(6-chloropyrimidin-4-yl)phenylcarbamate from step (c) above (0.088 g, 0.242 mmol) and 5-amino-2,3-dihydro-1H-inden-2-ol (0.048 g, 0.324 mmol, Example 85(a)) in DMSO (2 mL) was heated in a microwave synthesizer at 105° C. for 30 min, at 110° C. for 1.5 h, and at 110° C. for 1 h. The reaction mixture was partitioned between satd aqueous solution of NaHCO₃ (30 mL) and EtOAc (20 mL). The aqueous phase was extracted with EtOAc (2×20 mL). The EtOAc filtrate and extracts were combined, washed with water (2×30 mL) and brine (40 mL), dried over sodium sulfate, and filtered. The filtrate was evaporated in vacuo and the residue was purified by silica gel column chromatography (gradient: 50-100% EtOAc in hexanes) to afford of the title compound as off-white solid. MS (ESI, pos. ion) m/z: 475 [M+1].

### EXAMPLE 96

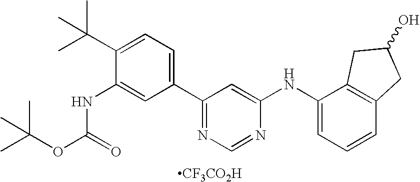

### tert-Butyl 2-tert-butyl-5-(6-(2-hydroxy-2,3-dihydro-1H-inden-4-ylamino)-pyrimidin-4-yl)phenylcarbamate, trifluoroacetic acid salt

A mixture of tert-butyl 2-tert-butyl-5-(6-chloropyrimidin-4-yl)phenylcarbamate (0.105 g, 0.289 mmol, Example 95(c)) and 4-amino-2,3-dihydro-1H-inden-2-ol (0.051 g, 0.344 mmol, Example 27(d)) in DMSO (2 mL) was heated in a microwave synthesizer at 110° C. for 1 h, and then at 120° C. for 1 h. The reaction mixture was partitioned between satd aqueous NaHCO₃ (40 mL) and EtOAc (20 mL). The aqueous phase was extracted with EtOAc (2×20 mL). The EtOAc filtrate and extracts were combined, washed with water (2×30 mL) and brine (30 mL), dried over sodium sulfate, and filtered. The filtrate was evaporated in vacuo and the residue was purified by preparative HPLC (gradient: 30%-80% CH₃CN (0.1% TFA)/H₂O (0.1% TFA) to give the title compound as a pale-yellow powder. MS (ESI, pos. ion) m/z: 475 [M+1].

### EXAMPLE 97

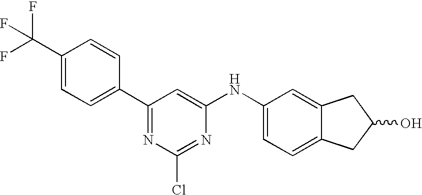

### 5-(2-Chloro-6-(4-(trifluoromethyl)phenyl)pyrimidin-4-ylamino)-2,3-dihydro-1H-inden-2-ol

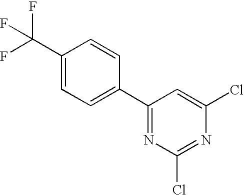 (a) 2,4-Dichloro-6-(4-(trifluoromethyl)phenyl)pyrimidine.

To a 20-mL, microwave vial was added 4-(trifluoromethyl)phenylboronic acid (9.0 g, 47.390 mmol, Aldrich), 2,4,6-trichloropyrimidine (5.45 mL, 47.4 mmol, Lancaster), palladium(II) acetate (0.532 g, 2.369 mmol, Strem), triphenylphosphine (1.243 g, 4.739 mmol, Aldrich), sodium carbonate (15.07 g, 142.2 mmol), and DME (6.0 mL). The vial was sealed and the reaction mixture was heated in the microwave synthesiser at 140° C. for 30 min. The reaction mixture was diluted with EtOAc and filtered through a pad of Celite®. The filter cake was washed with EtOAc. The combined filtrates were evaporated in vacuo, and the residue purified by silica gel column chromatography (gradient: 5-20% DCM/hexane) to give the title compound as a white solid. MS (ESI, pos. ion) m/z: 293 [M+1]. 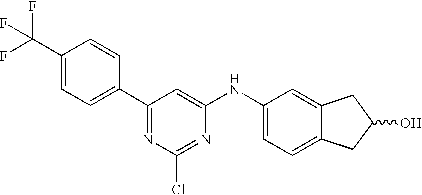
(b) 5-(2-Chloro-6-(4-(trifluoromethyl)phenyl)pyrimidin-4-ylamino)-2,3-dihydro-1H-inden-2-ol.

A mixture of 2,4-dichloro-6-(4-(trifluoromethyl)-phenyl)pyrimidine from step (a) above (0.150 g, 0.51 mmol) and 5-amino-2,3-dihydro-1H-inden-2-ol (0.076 g, 0.51 mmol, Example 85(a)) in DMSO (2 mL) was heated at 50° C. with stirring for 17 h. The reaction was allowed to reach room temperature, diluted with EtOAc (20 mL) and washed with water (2×20 mL). The EtOAc layer was washed with saturated NaCl (30 mL), dried over Na₂SO₄, and filtered. The filtrate was evaporated in vacuo and the residue was purified by silica gel column chromatography (gradient: 10-50% EtOAc/hexane) to give the title compound as a light-yellow solid. MS (ESI, pos. ion) m/z: 405 [M+1].

### EXAMPLE 98

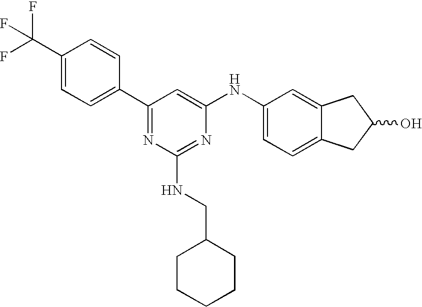

### 5-(2-(Cyclohexylmethylamino)-6-(4-(trifluoromethyl)phenyl)pyrimidin-4-ylamino)-2,3-dihydro-1H-inden-2-ol

A mixture of 5-(2-chloro-6-(4-(trifluoromethyl)phenyl)pyrimidin-4-ylamino)-2,3-dihydro-1H-inden-2-ol (0.080 g, 0.20 mmol, Example 97(b)) and aminomethylcyclohexane (0.067 g, 0.59 mmol, Aldrich) in DMSO (2 mL) was heated in a microwave synthesizer at 100° C. for 3 h. The reaction mixture was allowed to reach room temperature, and was diluted with EtOAc (20 mL), washed with water (2×30 mL) and brine (30 mL), dried over Na₂SO₄, and filtered. The filtrate was evaporated in vacuo and the residue was purified by silica gel column chromatography (gradient: 5-50% EtOAc/hexanes) to give the title compound as a pale-yellow solid. MS (ESI, pos. ion) m/z: 482 [M+1].

### EXAMPLE 99

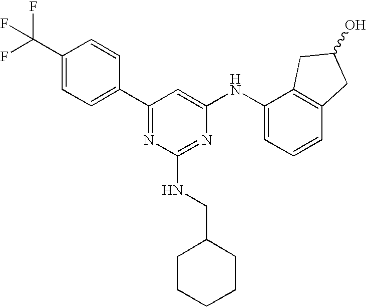

### 4-(2-(Cyclohexylmethylamino)-6-(4-(trifluoromethyl)phenyl)pyrimidin-4-ylamino)-2,3-dihydro-1H-inden-2-ol

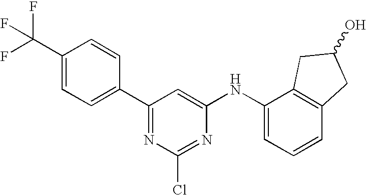 (a) 4-(2-Chloro-6-(4-(trifluoromethyl)phenyl)pyrimidin-4-ylamino)-2,3-dihydro-1H-inden-2-ol.

2,4-Dichloro-6-(4-(trifluoromethyl)phenyl)pyrimidine (0.16 g, 0.55 mmol, Example 97(a)) was reacted with 4-amino-2,3-dihydro-1H-inden-2-ol (0.082 g, 0.55 mmol, Example 27(c)) under the conditions of Example 97(b) to give the title compound as an off-white solid. MS (ESI, pos. ion) m/z: 405 [M+1]. 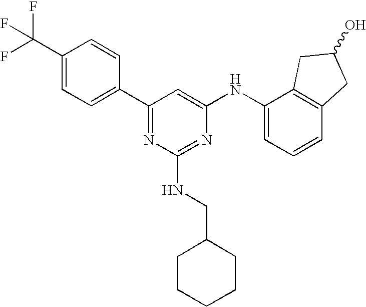
(b) 4-(2-(Cyclohexylmethylamino)-6-(4-(trifluoromethyl)phenyl)pyrimidin-4-ylamino)-2,3-dihydro-1H-inden-2-ol.

A mixture of 4-(2-chloro-6-(4-(trifluoromethyl)phenyl)pyrimidin-4-ylamino)-2,3-dihydro-1H-inden-2-ol from step (a) above (0.040 g, 0.099 mmol) and cyclohexanemethylamine (0.038 mL, 0.30 mmol, Aldrich) in DMSO (2 mL) was heated at 100° C. for 17 h. The reaction mixture was allowed to reach room temperature, and was diluted with EtOAc (20 mL), washed with water (2×30 mL) and brine (30 mL), dried over Na₂SO₄, and filtered. The filtrate was evaporated in vacuo and the residue was purified by silica gel column chromatography (gradient: 10-50% EtOAc/hexane) to give the title compound as a white solid. MS (ESI, pos. ion) m/z: 482 [M+1].

### EXAMPLE 100

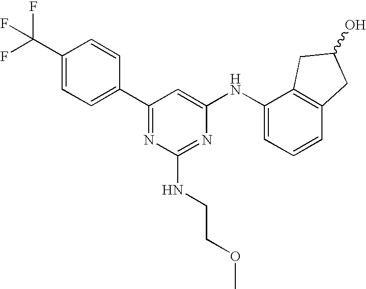

### 4-(2-(2-Methoxyethylamino)-6-(4-(trifluoromethyl)phenyl)pyrimidin-4-ylamino)-2,3-dihydro-1H-inden-2-ol

4-(2-Chloro-6-(4-(trifluoromethyl)-phenyl)pyrimidin-4-ylamino)-2,3-dihydro-1H-inden-2-ol (0.040 g, 0.099 mmol, Example 99(a)) was reacted with 2-methyoxyethylamine (0.026 mL, 0.30 mmol, Fluka) under the conditions of Example 99(b) to give the title compound as a white solid. MS (ESI, pos. ion) m/z: 444 [M+1].

Capsaicin-Induced Ca2+ Influx in Primary Dorsal Root Ganglion Neurons

Embryonic 19 day old (E19) dorsal root ganglia (DRG) were dissected from timed-pregnant, terminally anesthetized Sprague-Dawley rats (Charles River, Wilmington, Mass.) and collected in ice-cold L-15 media (Life Technologies, Grand Island, N.Y.) containing 5% heat inactivated horse serum (Life Technologies). The DRG were then dissociated into single cell suspension using a papain dissociation system (Worthington Biochemical Corp., Freehold, N.J.). The dissociated cells were pelleted at 200×g for 5 min and re-suspended in EBSS containing 1 mg/ml ovomucoid inhibitor, 1 mg/ml ovalbumin and 0.005% DNase. Cell suspension was centrifuged through a gradient solution containing 10 mg/ml ovomucoid inhibitor, 10 mg/ml ovalbumin at 200×g for 6 min to remove cell debris; and filtered through a 88-μm nylon mesh (Fisher Scientific, Pittsburgh, Pa.) to remove any clumps. Cell number was determined with a hemocytometer and cells were seeded into poly-ornithine 100 μg/ml (Sigma) and mouse laminin 1 μg/ml (Life Technologies)-coated 96-well plates at 10×10³ cells/well in complete medium. The complete medium consists of minimal essential medium (MEM) and Ham's F12, 1:1, penicillin (100 U/ml), and streptomycin (100 μg/ml), and nerve growth factor (10 ng/ml), 10% heat inactivated horse serum (Life Technologies). The cultures were kept at 37° C., 5% CO₂ and 100% humidity. For controlling the growth of non-neuronal cells, 5-fluoro-2′-deoxyuridine (75 μM) and uridine (180 μM) were included in the medium. Activation of VR1 is achieved in these cellular assays using either a capsaicin stimulus (ranging from 0.01-10 μM) or by an acid stimulus (addition of 30 mM Hepes/Mes buffered at pH 4.1). Compounds are also tested in an assay format to evaluate their agonist properties at VR1.

Capsaicin Antagonist Assay:

E-19 DRG cells at 5 days in culture are incubated with serial concentrations of VR1 antagonists, in HBSS (Hanks buffered saline solution supplemented with BSA 0.1 mg/ml and 1 mM Hepes at pH 7.4) for 15 min, 37° C. Cells are then challenged with a VR1 agonist, capsaicin 200 nM, in activation buffer containing 0.1 mg/ml BSA, 15 mM Hepes, pH 7.4, and 10 μCi/Ml⁴⁵Ca²⁺ (Amersham) in Ham's F12 for 2 min at 37° C.

The following compounds exhibit IC50 values of less than 5 mM in the Human VR1

Capsaicin Antagonist Assay:

(2R)-5-chloro-8-({6-[4-(trifluoromethyl)phenyl]pyrimidin-4-yl}amino)-1,2,3,4-tetrahydronaphthalen-2-ol;
(2R)-8-({6-[2-{[(1R)-1-(4-fluorophenyl)ethyl]amino}-6-(trifluoromethyl)pyridin-3-yl]pyrimidin-4-yl}amino)-1,2,3,4-tetrahydronaphthalen-2-ol;
(2R)-8-({6-[2-{[(1S)-1-(4-fluorophenyl)ethyl]amino}-6-(trifluoromethyl)pyridin-3-yl]pyrimidin-4-yl}amino)-1,2,3,4-tetrahydronaphthalen-2-ol;
(2R)-8-({6-[2-{[(1S)-2-(isopropylamino)-1-methylethyl]amino}-6-(trifluoromethyl)pyridin-3-yl]pyrimidin-4-yl}amino)-1,2,3,4-tetrahydronaphthalen-2-ol;
(2R)-8-({6-[2-{[(1S)-2-amino-1-methylethyl]amino}-6-(trifluoromethyl)pyridin-3-yl]pyrimidin-4-yl}amino)-1,2,3,4-tetrahydronaphthalen-2-ol;
(2R)-8-({6-[4-(trifluoromethyl)phenyl]pyrimidin-4-yl}amino)-1,2,3,4-tetrahydronaphthalen-2-ol;
(2R)-8-[(6-{4-[(1S)-1-(4-fluorophenyl)ethyl]piperazin-1-yl}pyrimidin-4-yl)-amino]-1,2,3,4-tetrahydronaphthalen-2-ol;
(2R,S)-4-[(6-{4-[(1R,S)-1-(4-fluorophenyl)ethyl]piperazin-1-yl}pyrimidin-4-yl)-amino]indan-2-ol;
(2R,S)-8-({6-[(2R,S)-2-(2-chlorophenyl)morpholin-4-yl]pyrimidin-4-yl}amino)-1,2,3,4-tetrahydronaphthalen-2-ol;
(2R,S)-8-({6-[2-{[(1R,S)-1-(4-fluorophenyl)ethyl]oxy}-4-(trifluoromethyl)-phenyl]pyrimidin-4-yl}amino)-1,2,3,4-tetrahydronaphthalen-2-ol;
(2R,S)-8-({6-[2-{[(2R,S)-tetrahydrofuran-2-ylmethyl]amino}-6-(trifluoromethyl)-pyridin-3-yl]pyrimidin-4-yl}amino)-1,2,3,4-tetrahydronaphthalen-2-ol;
(2S)-5-bromo-8-({6-[4-(trifluoromethyl)phenyl]pyrimidin-4-yl}amino)-1,2,3,4-tetrahydronaphthalen-2-ol;
(2S)-5-chloro-8-({5-chloro-6-[4-(trifluoromethyl)phenyl]pyrimidin-4-yl}amino)-1,2,3,4-tetrahydronaphthalen-2-ol;
(2S)-5-chloro-8-({6-[4-(trifluoromethyl)phenyl]pyrimidin-4-yl}amino)-1,2,3,4-tetrahydronaphthalen-2-ol;
(2S)-8-({6-[2-{[(1R)-1-(4-fluorophenyl)ethyl]amino}-6-(trifluoromethyl)pyridin-3-yl]pyrimidin-4-yl}amino)-1,2,3,4-tetrahydronaphthalen-2-ol;
(2S)-8-({6-[2-{[(1S)-1-(4-fluorophenyl)ethyl]amino}-6-(trifluoromethyl)pyridin-3-yl]pyrimidin-4-yl}amino)-1,2,3,4-tetrahydronaphthalen-2-ol;
(2S)-8-({6-[2-{[(1S)-2-(isopropylamino)-1-methylethyl]amino}-6-(trifluoromethyl)pyridin-3-yl]pyrimidin-4-yl}amino)-1,2,3,4-tetrahydronaphthalen-2-ol;
(2S)-8-({6-[2-{[(1S)-2-amino-1-methylethyl]amino}-6-(trifluoromethyl)pyridin-3-yl]pyrimidin-4-yl}amino)-1,2,3,4-tetrahydronaphthalen-2-ol;
(2S)-8-({6-[4-(trifluoromethyl)phenyl]pyrimidin-4-yl}amino)-1,2,3,4-tetrahydronaphthalen-2-ol;
(2S)-8-[(6-{4-[(1S)-1-(4-fluorophenyl)ethyl]piperazin-1-yl}pyrimidin-4-yl)-amino]-1,2,3,4-tetrahydronaphthalen-2-ol;
4-({6-[4-(trifluoromethyl)phenyl]pyrimidin-4-yl}amino)indan-2-ol;
5-({6-[2-amino-4-(trifluoromethyl)phenyl]pyrimidin-4-yl}amino)-1,2,3,4-tetrahydronaphthalene-2,3-cis-diol;
5-({6-[4-(trifluoromethyl)phenyl]pyrimidin-4-yl}amino)-1,2,3,4-tetrahydronaphthalene-2,3-cis-diol;
5-({6-[4-(trifluoromethyl)phenyl]pyrimidin-4-yl}amino)-1,2,3,4-tetrahydronaphthalene-2,3-trans-diol;
5-chloro-8-({6-[4-(trifluoromethyl)phenyl]pyrimidin-4-yl}amino)-1,2,3,4-tetrahydronaphthalen-2-ol;
6-fluoro-8-({6-[4-(trifluoromethyl)phenyl]pyrimidin-4-yl}amino)-1,2,3,4-tetrahydronaphthalen-2-ol;
7-chloro-8-({6-[4-(trifluoromethyl)phenyl]pyrimidin-4-yl}amino)-1,2,3,4-tetrahydronaphthalen-2-ol;
8-({2-chloro-6-[4-(trifluoromethyl)phenyl]pyrimidin-4-yl}amino)-1,2,3,4-tetrahydronaphthalen-2-ol;
8-({4-[4-(trifluoromethyl)phenyl]pyridin-2-yl}amino)-1,2,3,4-tetrahydro-naphthalen-2-ol;
8-({6-[2-(isobutylamino)-6-(trifluoromethyl)pyridin-3-yl]pyrimidin-4-yl}amino)-1,2,3,4-tetrahydronaphthalen-2-ol;
8-({6-[2-(isopropylamino)-6-(trifluoromethyl)pyridin-3-yl]pyrimidin-4-yl}amino)-1,2,3,4-tetrahydronaphthalen-2-ol;
8-({6-[2-(methylamino)-6-(trifluoromethyl)pyridin-3-yl]pyrimidin-4-yl}amino)-1,2,3,4-tetrahydronaphthalen-2-ol;
8-({6-[2-(piperidin-1-ylmethyl)-4-(trifluoromethyl)phenyl]pyrimidin-4-yl}amino)-1,2,3,4-tetrahydronaphthalen-2-ol;
8-({6-[2-[(1,1-dimethyl-2-morpholin-4-ylethyl)amino]-6-(trifluoromethyl)pyridin-3-yl]pyrimidin-4-yl}amino)-1,2,3,4-tetrahydronaphthalen-2-ol;
8-({6-[2-[(1-adamantylmethyl)amino]-6-(trifluoromethyl)pyridin-3-yl]pyrimidin-4-yl}amino)-1,2,3,4-tetrahydronaphthalen-2-ol;
8-({6-[2-[(2-methoxyethyl)amino]-6-(trifluoromethyl)pyridin-3-yl]pyrimidin-4-yl}amino)-1,2,3,4-tetrahydronaphthalen-2-ol;
8-({6-[2-[(cyclohexylmethyl)amino]-6-(trifluoromethyl)pyridin-3-yl]pyrimidin-4-yl}amino)-1,2,3,4-tetrahydronaphthalen-2-ol;
8-({6-[2-{[(1-hydroxycyclohexyl)methyl]amino}-6-(trifluoromethyl)pyridin-3-yl]pyrimidin-4-yl}amino)-1,2,3,4-tetrahydronaphthalen-2-ol;
8-({6-[2-amino-4-(trifluoromethyl)phenyl]pyrimidin-4-yl}amino)-1,2,3,4-tetrahydronaphthalen-2-ol;
8-({6-[2-amino-4-(trifluoromethyl)phenyl]pyrimidin-4-yl}amino)-5-chloro-1,2,3,4-tetrahydronaphthalen-2-ol;
8-({6-[2-amino-6-(trifluoromethyl)pyridin-3-yl]pyrimidin-4-yl}amino)-1,2,3,4-tetrahydronaphthalen-2-ol;
8-({6-[2-methoxy-6-(trifluoromethyl)pyridin-3-yl]pyrimidin-4-yl}amino)-1,2,3,4-tetrahydronaphthalen-2-ol;
8-({6-[2-piperidin-1-yl-6-(trifluoromethyl)pyridin-3-yl]pyrimidin-4-yl}amino)-1,2,3,4-tetrahydronaphthalen-2-ol;
8-({6-[2-pyrrolidin-1-yl-6-(trifluoromethyl)pyridin-3-yl]pyrimidin-4-yl}amino)-1,2,3,4-tetrahydronaphthalen-2-ol;
8-({6-[4-(trifluoromethyl)phenyl]pyrimidin-4-yl}amino)-1,2,3,4-tetrahydro-naphthalen-2-ol;
8-({6-[4-(trifluoromethyl)phenyl]pyrimidin-4-yl}amino)-2-naphthol;
8-[(6-{4-[(1R,S)-)-1-(4-fluorophenyl)ethyl]piperazin-1-yl}pyrimidin-4-yl)amino]-2-naphthol;
ethyl N-[3-(6-{[(7R)-7-hydroxy-5,6,7,8-tetrahydronaphthalen-1-yl]amino}pyrimidin-4-yl)-6-(trifluoromethyl)pyridin-2-yl]-L-phenylalaninate;
ethyl N-[3-(6-{[(7S)-7-hydroxy-5,6,7,8-tetrahydronaphthalen-1-yl]amino}pyrimidin-4-yl)-6-(trifluoromethyl)pyridin-2-yl]-L-phenylalaninate;
N-(5,6,7,8-tetrahydronaphthalen-1-yl)-6-[4-(trifluoromethyl)phenyl]pyrimidin-4-amine;
N-[2-(6-{[(7R)-7-hydroxy-5,6,7,8-tetrahydronaphthalen-1-yl]amino}pyrimidin-4-yl)-5-(trifluoromethyl)phenyl]-L-prolinamide;
N-[2-(6-{[(7S)-7-hydroxy-5,6,7,8-tetrahydronaphthalen-1-yl]amino}pyrimidin-4-yl)-5-(trifluoromethyl)phenyl]-L-prolinamide;
N-[2-(6-{[4-chloro-7-hydroxy-5,6,7,8-tetrahydronaphthalen-1-yl]amino}pyrimidin-4-yl)-5-(trifluoromethyl)phenyl]-3,3-dimethylbutanamide;
N-[2-(6-{[7-hydroxy-5,6,7,8-tetrahydronaphthalen-1-yl]amino}pyrimidin-4-yl)-5-(trifluoromethyl)phenyl]-3,3-dimethylbutanamide;
N-[2-(6-{[7-hydroxy-5,6,7,8-tetrahydronaphthalen-1-yl]amino}pyrimidin-4-yl)-5-(trifluoromethyl)phenyl]acetamide;
N-[3-(6-{[(7R)-7-hydroxy-5,6,7,8-tetrahydronaphthalen-1-yl]amino}pyrimidin-4-yl)-6-(trifluoromethyl)pyridin-2-yl]-L-phenylalanine;
N-[3-(6-{[(7S)-7-hydroxy-5,6,7,8-tetrahydronaphthalen-1-yl]amino}pyrimidin-4-yl)-6-(trifluoromethyl)pyridin-2-yl]-L-phenylalanine;
tert-butyl 2-(6-{[4-chloro-7-hydroxy-5,6,7,8-tetrahydronaphthalen-1-yl]amino}-pyrimidin-4-yl)-5-(trifluoromethyl)phenylcarbamate;
tert-butyl 2-(6-{[-7-hydroxy-5,6,7,8-tetrahydronaphthalen-1-yl]amino}pyrimidin-4-yl)-5-(trifluoromethyl)phenylcarbamate;
trans-3-methoxy-5-({6-[4-(trifluoromethyl)phenyl]pyrimidin-4-yl}amino)-1,2,3,4-tetrahydronaphthalen-2-ol; and
trans-3-methoxy-8-({6-[4-(trifluoromethyl)phenyl]pyrimidin-4-yl}amino)-1,2,3,4-tetrahydronaphthalen-2-ol.
   Acid Antagonist Assay:

Compounds are pre-incubated with E-19 DRG cells for 2 minutes prior to addition of Calcium-45 in 30 mM Hepes/Mes buffer (Final Assay pH 5) and then left for an additional 2 minutes prior to compound washout. Final 45 Ca (Amersham CES3-2 mCi) at 10 μCi/mL.

Agonist Assay:

Compounds are incubated with E-19 DRG cells for 2 minutes in the presence of Calcium-45 prior to compound washout. Final ⁴⁵Ca²⁺ (Amersham CES3-2 mCi) at 10 μCi/mL.

Compound Washout and Analysis:

Assay plates are washed using an ELX405 plate washer (Bio-Tek Instruments Inc.) immediately after functional assay. Wash 3× with PBS Mg2+/Ca2+ free, 0.1 mg/mL BSA. Aspirate between washes. Read plates using a MicroBeta Jet (Wallac Inc.). Compound activity is then calculated using appropriate computational algorithms.

⁴⁵Calcium²⁺ Assay Protocol

Compounds may be assayed using Chinese Hamster Ovary cell lines stably expressing either human VR1 or rat VR1 under a CMV promoter. Cells can be cultured in Growth Medium, routinely passaged at 70% confluency using trypsin and plated in the assay plate 24 hours prior to compound evaluation.

Possible Growth Medium:
DMEM, high glucose (Gibco 11965-084).
10% Dialyzed serum (Hyclone SH30079.03).
1× Non-Essential Amino Acids (Gibco 11140-050).
1× Glutamine-Pen-Strep (Gibco 10378-016).
Geneticin, 450 μg/mL (Gibco 10131-035).
   Compounds can be diluted in 100% DMSO and tested for activity over several log units of concentration [40 μM-2 pM]. Compounds may be further diluted in HBSS buffer (pH 7.4) 0.1 mg/mL BSA, prior to evaluation. Final DMSO concentration in assay would be 0.5%. Each assay plate can be controlled with a buffer only and a known antagonist compound (either capsazepine or one of the described VR1 antagonists).

Activation of VR1 can be achieved in these cellular assays using either a capsaicin stimulus (ranging from 0.1-1 μM) or by an acid stimulus (addition of 30 mM Hepes/Mes buffered at pH 4.1). Compounds may also tested in an assay format to evaluate their agonist properties at VR1.

Capsaicin Antagonist Assay:

Compounds may be pre-incubated with cells (expressing either human or rat VR1) for 2 minutes prior to addition of Calcium-45 and Capsaicin and then left for an additional 2 minutes prior to compound washout. Capsaicin (0.5 nM) can be added in HAM's F12, 0.1 mg/mL BSA, 15 mM Hepes at pH 7.4. Final ⁴⁵Ca (Amersham CES3-2 mCi) at 10 μCi/mL.

Acid Antagonist Assay:

Compounds can be pre-incubated with cells (expressing either human or rat VR1) for 2 minutes prior to addition of Calcium-45 in 30 mM Hepes/Mes buffer (Final Assay pH 5) and then left for an additional 2 minutes prior to compound washout. Final ⁴⁵Ca (Amersham CES3-2 mCi) at 10 μCi/mL.

Agonist Assay:

Compounds can be incubated with cells (expressing either human or rat VR1) for 2 minutes in the presence of Calcium-45 prior to compound washout. Final ⁴⁵Ca (Amersham CES3-2 mCi) at 10 μCi/mL.

Compound Washout and Analysis:

Assay plates can be washed using an ELX405 plate washer (Bio-Tek Instruments Inc.) immediately after functional assay. One can wash 3× with PBS Mg2⁺/Ca²⁺ free, 0.1 mg/mL BSA, aspirating between washes. Plates may be read using a MicroBeta Jet (Wallac Inc.). Compound activity may then calculated using appropriate computational algorithms.

Useful nucleic acid sequences and proteins may be found in U.S. Pat. Nos. 6,335,180, 6,406,908 and 6,239,267, herein incorporated by reference in their entirety.

For the treatment of vanilloid-receptor-diseases, such as acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders, the compounds of the present invention may be administered orally, parentally, by inhalation spray, rectally, or topically in dosage unit formulations containing conventional pharmaceutically acceptable carriers, adjuvants, and vehicles. The term parenteral as used herein includes, subcutaneous, intravenous, intramuscular, intrasternal, infusion techniques or intraperitoneally.

Treatment of diseases and disorders herein is intended to also include the prophylactic administration of a compound of the invention, a pharmaceutical salt thereof, or a pharmaceutical composition of either to a subject (i.e., an animal, preferably a mammal, most preferably a human) believed to be in need of preventative treatment, such as, for example, pain, inflammation and the like.

The dosage regimen for treating vanilloid-receptor-mediated diseases, cancer, and/or hyperglycemia with the compounds of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, preferably from about 0.1 mg to 10 mg/kg, more preferably from about 0.25 mg to 1 mg/kg are useful for all methods of use disclosed herein.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a capsule, a tablet, a suspension, or liquid. The pharmaceutical composition is preferably made in the form of a dosage unit containing a given amount of the active ingredient. For example, these may contain an amount of active ingredient from about 1 to 2000 mg, preferably from about 1 to 500 mg, more preferably from about 5 to 150 mg. A suitable daily dose for a human or other mammal may vary widely depending on the condition of the patient and other factors, but, once again, can be determined using routine methods.

The active ingredient may also be administered by injection as a composition with suitable carriers including saline, dextrose, or water. The daily parenteral dosage regimen will be from about 0.1 to about 30 mg/kg of total body weight, preferably from about 0.1 to about 10 mg/kg, and more preferably from about 0.25 mg to 1 mg/kg.

Injectable preparations, such as sterile injectable aqueous or oleaginous suspensions, may be formulated according to the known are using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

Formulations suitable for topical administration include liquid or semi-liquid preparations suitable for penetration through the skin (e.g., liniments, lotions, ointments, creams, or pastes) and drops suitable for administration to the eye, ear, or nose.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

Compounds of the present invention can possess one or more asymmetric carbon atoms and are thus capable of existing in the form of optical isomers as well as in the form of racemic or non-racemic mixtures thereof. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using active starting materials. These isomers may be in the form of a free acid, a free base, an ester or a salt.

Likewise, the compounds of this invention may exist as isomers, that is compounds of the same molecular formula but in which the atoms, relative to one another, are arranged differently. In particular, the alkylene substituents of the compounds of this invention, are normally and preferably arranged and inserted into the molecules as indicated in the definitions for each of these groups, being read from left to right. However, in certain cases, one skilled in the art will appreciate that it is possible to prepare compounds of this invention in which these substituents are reversed in orientation relative to the other atoms in the molecule. That is, the substituent to be inserted may be the same as that noted above except that it is inserted into the molecule in the reverse orientation. One skilled in the art will appreciate that these isomeric forms of the compounds of this invention are to be construed as encompassed within the scope of the present invention.

The compounds of the present invention can be used in the form of salts derived from inorganic or organic acids. The salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methansulfonate, nicotinate, 2-naphthalenesulfonate, oxalate, palmoate, pectinate, persulfate, 2-phenylpropionate, picrate, pivalate, propionate, succinate, tartrate, thiocyanate, tosylate, mesylate, and undecanoate. Also, the basic nitrogen-containing groups can be quaternized with such agents as lower alkyl halides, such as methyl, ethyl, propyl, and butyl chloride, bromides and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids that may be employed to from pharmaceutically acceptable acid addition salts include such inorganic acids as hydrochloric acid, sulfuric acid and phosphoric acid and such organic acids as oxalic acid, maleic acid, succinic acid and citric acid. Other examples include salts with alkali metals or alkaline earth metals, such as sodium, potassium, calcium or magnesium or with organic bases.

Also encompassed in the scope of the present invention are pharmaceutically acceptable esters of a carboxylic acid or hydroxyl containing group, including a metabolically labile ester or a prodrug form of a compound of this invention. A metabolically labile ester is one which may produce, for example, an increase in blood levels and prolong the efficacy of the corresponding non-esterified form of the compound. A prodrug form is one which is not in an active form of the molecule as administered but which becomes therapeutically active after some in vivo activity or biotransformation, such as metabolism, for example, enzymatic or hydrolytic cleavage. For a general discussion of prodrugs involving esters see Svensson and Tunek Drug Metabolism Reviews 165 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 039,051 (Sloan and Little, Apr. 11, 1981) discloses Mannich-base hydroxamic acid prodrugs, their preparation and use. Esters of a compound of this invention, may include, for example, the methyl, ethyl, propyl, and butyl esters, as well as other suitable esters formed between an acidic moiety and a hydroxyl containing moiety. Metabolically labile esters, may include, for example, methoxymethyl, ethoxymethyl, iso-propoxymethyl, α-methoxyethyl, groups such as α-((C₁-C₄)-alkyloxy)ethyl, for example, methoxyethyl, ethoxyethyl, propoxyethyl, iso-propoxyethyl, etc.; 2-oxo-1,3-dioxolen-4-ylmethyl groups, such as 5-methyl-2-oxo-1,3,dioxolen-4-ylmethyl, etc.; C₁-C₃ alkylthiomethyl groups, for example, methylthiomethyl, ethylthiomethyl, isopropylthiomethyl, etc.; acyloxymethyl groups, for example, pivaloyloxymethyl, α-acetoxymethyl, etc.; ethoxycarbonyl-1-methyl; or α-acyloxy-α-substituted methyl groups, for example α-acetoxyethyl.

Further, the compounds of the invention may exist as crystalline solids which can be crystallized from common solvents such as ethanol, N,N-dimethyl-formamide, water, or the like. Thus, crystalline forms of the compounds of the invention may exist as polymorphs, solvates and/or hydrates of the parent compounds or their pharmaceutically acceptable salts. All of such forms likewise are to be construed as falling within the scope of the invention.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The foregoing is merely illustrative of the invention and is not intended to limit the invention to the disclosed compounds. Variations and changes which are obvious to one skilled in the art are intended to be within the scope and nature of the invention which are defined in the appended claims.

From the foregoing description, one skilled in the art can easily ascertain the essential characteristics of this invention, and without departing from the spirit and scope thereof, can make various changes and modifications of the invention to adapt it to various usages and conditions.

We Claim:

1. A compound having the structure: 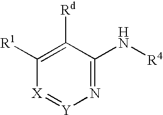
or any pharmaceutically-acceptable salt thereof, wherein:
X is N and Y is C(R³); or X is C(R²) and Y is N;
n is independently, at each instance, 0, 1 or 2;
R¹ is 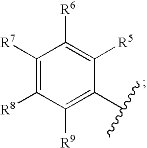
or R¹ is R^{b} substituted by 0, 1, 2 or 3 substituents selected from R^{e}, R^{h}, —OR^{f}, —OR^{h}, —OC₂₋₆alkylNR^{a}R^{f}, —OC₂₋₆alkylOR^{f}, —NR^{a}R^{f}, —NR^{a}R^{h}, —NR^{a}C₂₋₆alkylNR^{a}R^{f}, —NR^{a}C₂₋₆alkylOR^{f}, —CO₂R^{e}, —OC(═O)R^{e}, —C(═O)R^{e}, —C(═O)NR^{a}R^{f}, —C(═O)NR^{a}R^{h}, —NR^{a}C(═O)R^{e}, —NR^{a}C(═O)R^{h}, —NR^{a}C(═O)NR^{a}R^{f}, —NR^{a}CO₂R^{f}, —C₁₋₈alkylOR^{f}, —C₁₋₆alkylNR^{a}R^{f}, —S(═O)ₙR^{e}, —S(═O)₂NR^{a}R^{f}, —NR^{a}S(═O)₂R^{e}, —OS(═O)₂R^{e}, —OC(═O)NR^{a}R^{f}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —NR^{e}C(═O)R^{a}, —NR^{h}C(═O)R^{a}, —NR^{h}C(═O)NR^{a}R^{a}, −NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} and —OC(═O)NR^{a}R^{h};
R² is selected from H, halo, cyano, nitro, Rⁱ, R^{k}, —OH, —ORⁱ, —OR^{k}, —S(O)ₙRⁱ, —S(O)ₙR^{k}, —N(R^{a})S(O)ₙRⁱ, —N(R^{a})S(O)ₙR^{k}, —S(O)ₙN(R^{a})Rⁱ, —S(O)ₙN(R^{a})R^{k}, —NH₂, —NR^{a}Rⁱ and —NR^{a}R^{k};
R³ is selected from H, halo, —NH₂, —NHC₁₋₃alkyl, —N(C₁₋₃alkyl)C₁₋₃alkyl, or C₁₋₃alkyl;
R⁴ is 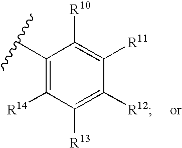
R⁴ is naphthyl substituted by OH, NH₂ or NHC₁₋₆alkyl, and additionally substituted by 0, 1, 2 or 3 substituents independently selected from R^{k}, C₁₋₄haloalkyl, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a}; and wherein the naphthyl is additionally substituted by 0 or 1 groups independently selected from Rⁱ and additionally substituted by 0, 1, 2, 3, 4 or 5 substituents independently selected from Br, Cl, F and I;
R⁵ is independently, at each instance, H, R^{k}, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a};
R⁶ is independently, at each instance, H, R^{k}, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a};
R⁷ is independently, at each instance, H, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h};
R⁸ is independently, at each instance, H, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h};
R⁹ is independently, at each instance, H, C₁₋₄haloalkyl, halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a}, R^{e}, R^{h}, —OR^{e}, —OR^{h}, —OC₂₋₆alkylNR^{a}R^{e}, —OC₂₋₆alkylOR^{e}, —NR^{a}R^{e}, —NR^{a}R^{h}, —NR^{a}C₂₋₆alkylNR^{a}R^{e}, —NR^{a}C₂₋₆alkylOR^{e}, —CO₂R^{e}, —OC(═O)R^{e}, —C(═O)R^{e}, —C(═O)NR^{a}R^{e}, —C(═O)NR^{a}R^{h}, —NR^{a}C(═O)R^{e}, —NR^{a}C(═O)R^{h}, —NR^{a}C(═O)NR^{a}R^{e}, —NR^{a}CO₂R^{e}, —C₁₋₈alkylOR^{e}, —C₁₋₆alkylNR^{a}R^{e}, —S(═O)ₙR^{e}, —S(═O)₂NR^{a}R^{e}, —NR^{a}S(═O)₂R^{e}, —OS(═O)₂R^{e}, —OC(═O)NR^{a}R^{e}, —OR^{h}, —OC₂₋₆alkylNR^{a}R^{h}, —OC₂₋₆alkylOR^{h}, —NR^{a}C₂₋₆alkylNR^{a}R^{h}, —NR^{h}C₂₋₆alkylNR^{a}R^{a}, —NR^{h}C₂₋₆alkylOR^{a}, —NR^{a}C₂₋₆alkylOR^{h}, —CO₂R^{h}, —OC(═O)R^{h}, —C(═O)R^{h}, —C(═O)NR^{a}R^{h}, —NR^{e}C(═O)R^{a}, —NR^{h}C(═O)R^{a}, —NR^{h}C(═O)NR^{a}R^{a}, —NR^{e}C(═O)NR^{a}R^{a}, —NR^{h}CO₂R^{a}, —NR^{e}CO₂R^{a}, —C₁₋₈alkylOR^{h}, —C₁₋₆alkylNR^{a}R^{h}, —S(═O)ₙR^{h}, —S(═O)₂NR^{a}R^{h}, —NR^{a}S(═O)₂R^{h}, —NR^{h}S(═O)₂R^{a}, —OS(═O)₂R^{h} or —OC(═O)NR^{a}R^{h}; and
A)
R¹⁰ and R¹¹ together are a saturated or partially unsaturated 3-, 4- or 5-carbon bridge, wherein the bridge is substituted by 0, 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; and
R¹² is independently, at each instance, selected from H, C₁₋₈alkyl, C₁₋₄haloalkyl, halo, cyano, nitro, —OH, —NH₂, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹² is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹² is C₁₋₄alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═C)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ, and additionally substituted by 0, 1 or 2 halo groups; or
B)
R¹⁰ is independently, at each instance, selected from H, halo, C₁₋₈alkyl, C₁₋₄haloalkyl, cyano, nitro, —C(═O)R^{k}, —C(═O)OR^{k}, —C(═O)NR^{a}R^{k}, —C(═NR^{a})NR^{a}R^{k}, —OH, —NH₂, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁰ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein there are no more than 2 N atoms, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁰ is C₁₋₄alkyl substituted by 0, 1, 2 or 3 groups selected from C₁₋₄haloalkyl, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; and
R¹¹ and R¹² together are a saturated or partially unsaturated 3-, 4- or 5-carbon bridge, wherein the bridge is substituted by 0, 1 or 2 substituents selected from oxo, thioxo, Rⁱ, R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ;
R¹³ is independently, at each instance, selected from H, halo, cyano, nitro, C₁₋₄haloalkyl, —OH, —NH₂, C₁₋₈alkyl, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹³ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)Rk, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹³ is C₁₋₄alkyl substituted by 0, 1, 2 or 3 groups selected from C₁₋₄haloalkyl, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ;
R¹⁴ is independently, at each instance, selected from H, C₁₋₈alkyl, C₁₋₄haloalkyl, halo, cyano, nitro, —OH, —NH₂, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁴ is a saturated, partially-saturated or unsaturated 5-, 6- or 7-membered monocyclic or 6-, 7-, 8-, 9-, 10- or 11-membered bicyclic ring containing 1, 2 or 3 atoms selected from N, O and S, wherein there are no more than 2 N atoms, wherein the ring is substituted by 0, 1 or 2 oxo or thioxo groups, wherein the ring is substituted by 0, 1, 2 or 3 groups selected from R^{k}, halo, cyano, nitro, —C(═O)R^{k}, —C(═O)OR^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ; or R¹⁴ is C₁₋₄alkyl substituted by 0, 1, 2 or 3 groups selected from C₁₋₄haloalkyl, halo, cyano, nitro, —C(═O)R^{k}, —C(═O)NR^{a}R^{k}, —C(═NR^{a})NR^{a}R^{k}, —OR^{k}, —OC(═O)R^{k}, —OC(═O)NR^{a}R^{k}, —OC(═O)N(R^{a})S(═O)₂R^{k}, —OC₂₋₆alkylNR^{a}R^{k}, —OC₂₋₆alkylOR^{k}, —SR^{k}, —S(═O)R^{k}, —S(═O)₂R^{k}, —S(═O)₂NR^{a}R^{k}, —S(═O)₂N(R^{a})C(═O)R^{k}, —S(═O)₂N(R^{a})C(═O)OR^{k}, —S(═O)₂N(R^{a})C(═O)NR^{a}R^{k}, —NR^{a}R^{k}, —N(R^{a})C(═O)R^{k}, —N(R^{a})C(═O)OR^{k}, —N(R^{a})C(═O)NR^{a}R^{k}, —N(R^{a})C(═NR^{a})NR^{a}R^{k}, —N(R^{a})S(═O)₂R^{k}, —N(R^{a})S(═O)₂NR^{a}R^{k}, —NR^{a}C₂₋₆alkylNR^{a}R^{k}, —NR^{a}C₂₋₆alkylOR^{k}, —C(═O)Rⁱ, —C(═O)ORⁱ, —C(═O)NR^{a}Rⁱ, —C(═NR^{a})NR^{a}Rⁱ, —ORⁱ, —OC(═O)Rⁱ, —OC(═O)NR^{a}Rⁱ, —OC(═O)N(R^{a})S(═O)₂Rⁱ, —OC(═O)N(Rⁱ)S(═O)₂R^{k}, —OC₂₋₆alkylNR^{a}Rⁱ, —OC₂₋₆alkylORⁱ, —SRⁱ, —S(═O)Rⁱ, —S(═O)₂Rⁱ, —S(═O)₂NR^{a}Rⁱ, —S(═O)₂N(Rⁱ)C(═O)R^{k}, —S(═O)₂N(R^{a})C(═O)Rⁱ, —S(═O)₂N(Rⁱ)C(═O)OR^{k}, —S(═O)₂N(R^{a})C(═O)ORⁱ, —S(═O)₂N(Rⁱ)C(═O)NR^{a}R^{k}, —S(═O)₂N(R^{a})C(═O)NR^{a}Rⁱ, —NR^{a}Rⁱ, —N(Rⁱ)C(═O)R^{k}, —N(R^{a})C(═O)Rⁱ, —N(Rⁱ)C(═O)OR^{k}, —N(R^{a})C(═O)ORⁱ, —N(Rⁱ)C(═O)NR^{a}R^{k}, —N(R^{a})C(═O)NR^{a}Rⁱ, —N(Rⁱ)C(═NR^{a})NR^{a}R^{k}, —N(R^{a})C(═NR^{a})NR^{a}Rⁱ, —N(Rⁱ)S(═O)₂R^{k}, —N(R^{a})S(═O)₂Rⁱ, —N(Rⁱ)S(═O)₂NR^{a}R^{k}, —N(R^{a})S(═O)₂NR^{a}Rⁱ, —NRⁱC₂₋₆alkylNR^{a}R^{k}, —NR^{a}C₂₋₆alkylNR^{a}Rⁱ, —NRⁱC₂₋₆alkylOR^{k} and —NR^{a}C₂₋₆alkylORⁱ;
R^{a} is independently, at each instance, H, phenyl, benzyl or C₁₋₆alkyl, the phenyl, benzyl and C₁₋₆alkyl being substituted by 0, 1, 2 or 3 substituents selected from halo, C₁₋₄alkyl, C₁₋₃haloalkyl, —OC₁₋₄alkyl, —NH₂, —NHC₁₋₄alkyl, —N(C₁₋₄alkyl)C₁₋₄alkyl;
R^{b} is a saturated or partially saturated 5-, 6- or 7-membered monocyclic or 6-, 7-, 8-, 9-, 10- or 11-membered bicyclic carbocyclic ring, or a saturated, partially saturated or unsaturated 5-, 6- or 7-membered monocyclic or 6-, 7-, 8-, 9-, 10- or 11-membered bicyclic ring containing 1, 2, 3 or 4 atoms selected from N, O and S, wherein the carbon atoms of any ring are substituted by 0, 1 or 2 oxo or thioxo groups, sulfur atoms of the ring are substituted by 0, 1 or 2 oxo groups, nitrogen atoms of the ring are substituted by 0 or 1 oxo groups;
R^{d} is independently in each instance hydrogen or —CH₃;
R^{e} is, independently, in each instance, C₁₋₉alkyl substituted by 0 or 1 groups independently selected from R^{h}; and wherein the C₁₋₉alkyl is additionally substituted by 0, 1, 2 or 3 substituents selected from halo, C₁₋₄haloalkyl, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a};
R^{f} is, independently, in each instance, H or R^{e};
R^{h} is, independently, in each instance, phenyl or a saturated, partially-saturated or unsaturated 5- or 6-membered monocyclic ring containing 1, 2 or 3 atoms selected from N, O and S, so long as the combination of O and S atoms is not greater than 2, wherein the ring is substituted by 0 or 1 oxo or thioxo groups, wherein the phenyl and monocycle are substituted by 0, 1 or 2 substituents selected from halo, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a};
Rⁱ is a saturated, partially saturated or unsaturated 5-, 6- or 7-membered monocyclic or 6-, 7-, 8-, 9-, 10- or 11-membered bicyclic ring containing 0, 1, 2, 3 or 4 atoms selected from N, O and S, wherein the available carbon atoms of the ring are substituted by 0, 1 or 2 oxo or thioxo groups, wherein the ring is substituted by 0, 1, 2 or 3 substituents independently selected from C₁₋₉alkyl, halo, cyano, nitro, C₁₋₄haloalkyl, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a}, —NR^{a}C₂₋₆alkylOR^{a}, and C₁₋₉alkyl substituted by 1, 2 or 3 substituents selected from halo, cyano, nitro, C₁₋₄haloalkyl, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a}; and
R^{k} is, independently, in each instance, C₁₋₉alkyl or C₁₋₄alkyl(phenyl) wherein either is substituted by 0, 1, 2, 3 or 4 substituents selected from halo, C₁₋₄haloalkyl, cyano, nitro, —C(═O)R^{a}, —C(═O)OR^{a}, —C(═O)NR^{a}R^{a}, —C(═NR^{a})NR^{a}R^{a}, —OR^{a}, —OC(═O)R^{a}, —OC(═O)NR^{a}R^{a}, —OC(═O)N(R^{a})S(═O)₂R^{a}, —OC₂₋₆alkylNR^{a}R^{a}, —OC₂₋₆alkylOR^{a}, —SR^{a}, —S(═O)R^{a}, —S(═O)₂R^{a}, —S(═O)₂NR^{a}R^{a}, —S(═O)₂N(R^{a})C(═O)R^{a}, —S(═O)₂N(R^{a})C(═O)OR^{a}, —S(═O)₂N(R^{a})C(═O)NR^{a}R^{a}, —NR^{a}R^{a}, —N(R^{a})C(═O)R^{a}, —N(R^{a})C(═O)OR^{a}, —N(R^{a})C(═O)NR^{a}R^{a}, —N(R^{a})C(═NR^{a})NR^{a}R^{a}, —N(R^{a})S(═O)₂R^{a}, —N(R^{a})S(═O)₂NR^{a}R^{a}, —NR^{a}C₂₋₆alkylNR^{a}R^{a} and —NR^{a}C₂₋₆alkylOR^{a}; and wherein the C₁₋₉alkyl is additionally substituted by 0 or 1 groups independently selected from Rⁱ and additionally substituted by 0, 1, 2, 3, 4 or 5 substituents independently selected from Br, Cl, F and I.

2. A compound according to claim 1 that is selected from the group of:
(2R)-5-chloro-8-({6-[4-(trifluoromethyl)phenyl]pyrimidin-4-yl}amino)-1,2,3,4-tetrahydronaphthalen-2-ol;
(2R)-8-({6-[2-{[(1R)-1-(4-fluorophenyl)ethyl]amino}-6-(trifluoromethyl)pyridin-3-yl]pyrimidin-4-yl}amino)-1,2,3,4-tetrahydronaphthalen-2-ol;
(2R)-8-({6-[2-{[(1S)-1-(4-fluorophenyl)ethyl]amino}-6-(trifluoromethyl)pyridin-3-yl]pyrimidin-4-yl}amino)-1,2,3,4-tetrahydronaphthalen-2-ol;
(2R)-8-({6-[2-{[(1S)-2-(isopropylamino)-1-methylethyl]amino}-6-(trifluoromethyl)pyridin-3-yl]pyrimidin-4-yl}amino)-1,2,3,4-tetrahydro-naphthalen-2-ol;
(2R)-8-({6-[2-{[(1S)-2-amino-1-methylethyl]amino}-6-(trifluoromethyl)pyridin-3-yl]pyrimidin-4-yl}amino)-1,2,3,4-tetrahydronaphthalen-2-ol;
(2R)-8-({6-[4-(trifluoromethyl)phenyl]pyrimidin-4-yl}amino)-1,2,3,4-tetrahydro-naphthalen-2-ol;
(2R)-8-[(6-{4-[(1S)-1-(4-fluorophenyl)ethyl]piperazin-1-yl}pyrimidin-4-yl)-amino]-1,2,3,4-tetrahydronaphthalen-2-ol;
(2R,S)-4-[(6-{4-[(1R,S)-1-(4-fluorophenyl)ethyl]piperazin-1-yl}pyrimidin-4-yl)-amino]indan-2-ol;
(2R,S)-8-({6-[(2R,S)-2-(2-chlorophenyl)morpholin-4-yl]pyrimidin-4-yl}amino)-1,2,3,4-tetrahydronaphthalen-2-ol;
(2R,S)-8-({6-[2-{[(1R,S)-1-(4-fluorophenyl)ethyl]oxy}-4-(trifluoromethyl)-phenyl]pyrimidin-4-yl}amino)-1,2,3,4-tetrahydronaphthalen-2-ol;
(2R,S)-8-({6-[2-{[(2R,S)-tetrahydrofuran-2-ylmethyl]amino}-6-(trifluoromethyl)-pyridin-3-yl]pyrimidin-4-yl}amino)-1,2,3,4-tetrahydronaphthalen-2-ol;
(2S)-5-bromo-8-({6-[4-(trifluoromethyl)phenyl]pyrimidin-4-yl}amino)-1,2,3,4-tetrahydronaphthalen-2-ol;
(2S)-5-chloro-8-({5-chloro-6-[4-(trifluoromethyl)phenyl]pyrimidin-4-yl}amino)-1,2,3,4-tetrahydronaphthalen-2-ol;
(2S)-5-chloro-8-({6-[4-(trifluoromethyl)phenyl]pyrimidin-4-yl}amino)-1,2,3,4-tetrahydronaphthalen-2-ol;
(2S)-8-({6-[2-{[(1R)-1-(4-fluorophenyl)ethyl]amino}-6-(trifluoromethyl)pyridin-3-yl]pyrimidin-4-yl}amino)-1,2,3,4-tetrahydronaphthalen-2-ol;
(2S)-8-({6-[2-{[(1S)-1-(4-fluorophenyl)ethyl]amino}-6-(trifluoromethyl)pyridin-3-yl]pyrimidin-4-yl}amino)-1,2,3,4-tetrahydronaphthalen-2-ol;
(2S)-8-({6-[2-{[(1S)-2-(isopropylamino)-1-methylethyl]amino}-6-(trifluoromethyl)pyridin-3-yl]pyrimidin-4-yl}amino)-1,2,3,4-tetrahydro-naphthalen-2-ol;
(2S)-8-({6-[2-{[(1S)-2-amino-1-methylethyl]amino}-6-(trifluoromethyl)pyridin-3-yl]pyrimidin-4-yl}amino)-1,2,3,4-tetrahydronaphthalen-2-ol;
(2S)-8-({6-[4-(trifluoromethyl)phenyl]pyrimidin-4-yl}amino)-1,2,3,4-tetrahydro-naphthalen-2-ol;
(2S)-8-[(6-{4-[(1S)-1-(4-fluorophenyl)ethyl]piperazin-1-yl}pyrimidin-4-yl)-amino]-1,2,3,4-tetrahydronaphthalen-2-ol;
4-({6-[4-(trifluoromethyl)phenyl]pyrimidin-4-yl}amino)indan-2-ol;
5-({6-[2-amino-4-(trifluoromethyl)phenyl]pyrimidin-4-yl}amino)-1,2,3,4-tetrahydronaphthalene-2,3-cis-diol;
5-({6-[4-(trifluoromethyl)phenyl]pyrimidin-4-yl}amino)-1,2,3,4-tetrahydro-naphthalene-2,3-cis-diol;
5-({6-[4-(trifluoromethyl)phenyl]pyrimidin-4-yl}amino)-1,2,3,4-tetrahydro-naphthalene-2,3-trans-diol;
5-chloro-8-({6-[4-(trifluoromethyl)phenyl]pyrimidin-4-yl}amino)-1,2,3,4-tetrahydronaphthalen-2-ol;
6-fluoro-8-({6-[4-(trifluoromethyl)phenyl]pyrimidin-4-yl}amino)-1,2,3,4-tetrahydronaphthalen-2-ol;
7-chloro-8-({6-[4-(trifluoromethyl)phenyl]pyrimidin-4-yl}amino)-1,2,3,4-tetrahydronaphthalen-2-ol;
8-({2-chloro-6-[4-(trifluoromethyl)phenyl]pyrimidin-4-yl}amino)-1,2,3,4-tetrahydronaphthalen-2-ol;
8-({4-[4-(trifluoromethyl)phenyl]pyridin-2-yl}amino)-1,2,3,4-tetrahydro-naphthalen-2-ol;
8-({6-[2-(isobutylamino)-6-(trifluoromethyl)pyridin-3-yl]pyrimidin-4-yl}amino)-1,2,3,4-tetrahydronaphthalen-2-ol;
8-({6-[2-(isopropylamino)-6-(trifluoromethyl)pyridin-3-yl]pyrimidin-4-yl}amino)-1,2,3,4-tetrahydronaphthalen-2-ol;
8-({6-[2-(methylamino)-6-(trifluoromethyl)pyridin-3-yl]pyrimidin-4-yl}amino)-1,2,3,4-tetrahydronaphthalen-2-ol;
8-({6-[2-(piperidin-1-ylmethyl)-4-(trifluoromethyl)phenyl]pyrimidin-4-yl}amino)-1,2,3,4-tetrahydronaphthalen-2-ol;
8-({6-[2-[(1,1-dimethyl-2-morpholin-4-ylethyl)amino]-6-(trifluoromethyl)pyridin-3-yl]pyrimidin-4-yl}amino)-1,2,3,4-tetrahydronaphthalen-2-ol;
8-({6-[2-[(1-adamantylmethyl)amino]-6-(trifluoromethyl)pyridin-3-yl]pyrimidin-4-yl}amino)-1,2,3,4-tetrahydronaphthalen-2-ol;
8-({6-[2-[(2-methoxyethyl)amino]-6-(trifluoromethyl)pyridin-3-yl]pyrimidin-4-yl}amino)-1,2,3,4-tetrahydronaphthalen-2-ol;
8-({6-[2-[(cyclohexylmethyl)amino]-6-(trifluoromethyl)pyridin-3-yl]pyrimidin-4-yl}amino)-1,2,3,4-tetrahydronaphthalen-2-ol;
8-({6-[2-{[(1-hydroxycyclohexyl)methyl]amino}-6-(trifluoromethyl)pyridin-3-yl]pyrimidin-4-yl}amino)-1,2,3,4-tetrahydronaphthalen-2-ol;
8-({6-[2-amino-4-(trifluoromethyl)phenyl]pyrimidin-4-yl}amino)-1,2,3,4-tetrahydronaphthalen-2-ol;
8-({6-[2-amino-4-(trifluoromethyl)phenyl]pyrimidin-4-yl}amino)-5-chloro-1,2,3,4-tetrahydronaphthalen-2-ol;
8-({6-[2-amino-6-(trifluoromethyl)pyridin-3-yl]pyrimidin-4-yl}amino)-1,2,3,4-tetrahydronaphthalen-2-ol;
8-({6-[2-methoxy-6-(trifluoromethyl)pyridin-3-yl]pyrimidin-4-yl}amino)-1,2,3,4-tetrahydronaphthalen-2-ol;
8-({6-[2-piperidin-1-yl-6-(trifluoromethyl)pyridin-3-yl]pyrimidin-4-yl}amino)-1,2,3,4-tetrahydronaphthalen-2-ol;
8-({6-[2-pyrrolidin-1-yl-6-(trifluoromethyl)pyridin-3-yl]pyrimidin-4-yl}amino)-1,2,3,4-tetrahydronaphthalen-2-ol;
8-({6-[4-(trifluoromethyl)phenyl]pyrimidin-4-yl}amino)-1,2,3,4-tetrahydro-naphthalen-2-ol;
8-({6-[4-(trifluoromethyl)phenyl]pyrimidin-4-yl}amino)-2-naphthol;
8-[(6-{4-[(1R,S)-)-1-(4-fluorophenyl)ethyl]piperazin-1-yl}pyrimidin-4-yl)amino]-2-naphthol;
ethyl N-[3-(6-{[(7R)-7-hydroxy-5,6,7,8-tetrahydronaphthalen-1-yl]amino}pyrimidin-4-yl)-6-(trifluoromethyl)pyridin-2-yl]-L-phenylalaninate;
ethyl N-[3-(6-{[(7S)-7-hydroxy-5,6,7,8-tetrahydronaphthalen-1-yl]amino}pyrimidin-4-yl)-6-(trifluoromethyl)pyridin-2-yl]-L-phenylalaninate;
N-(5,6,7,8-tetrahydronaphthalen-1-yl)-6-[4-(trifluoromethyl)phenyl]pyrimidin-4-amine;
N-[2-(6-{[(7R)-7-hydroxy-5,6,7,8-tetrahydronaphthalen-1-yl]amino}pyrimidin-4-yl)-5-(trifluoromethyl)phenyl]-L-prolinamide;
N-[2-(6-{[(7S)-7-hydroxy-5,6,7,8-tetrahydronaphthalen-1-yl]amino}pyrimidin-4-yl)-5-(trifluoromethyl)phenyl]-L-prolinamide;
N-[2-(6-{[4-chloro-7-hydroxy-5,6,7,8-tetrahydronaphthalen-1-yl]amino}pyrimidin-4-yl)-5-(trifluoromethyl)phenyl]-3,3-dimethylbutanamide;
N-[2-(6-{[7-hydroxy-5,6,7,8-tetrahydronaphthalen-1-yl]amino}pyrimidin-4-yl)-5-(trifluoromethyl)phenyl]-3,3-dimethylbutanamide;
N-[2-(6-{[7-hydroxy-5,6,7,8-tetrahydronaphthalen-1-yl]amino}pyrimidin-4-yl)-5-(trifluoromethyl)phenyl]acetamide;
N-[3-(6-{[(7R)-7-hydroxy-5,6,7,8-tetrahydronaphthalen-1-yl]amino}pyrimidin-4-yl)-6-(trifluoromethyl)pyridin-2-yl]-L-phenylalanine;
N-[3-(6-{[(7S)-7-hydroxy-5,6,7,8-tetrahydronaphthalen-1-yl]amino}pyrimidin-4-yl)-6-(trifluoromethyl)pyridin-2-yl]-L-phenylalanine;
tert-butyl 2-(6-{[4-chloro-7-hydroxy-5,6,7,8-tetrahydronaphthalen-1-yl]amino}-pyrimidin-4-yl)-5-(trifluoromethyl)phenylcarbamate;
tert-butyl 2-(6-{[-7-hydroxy-5,6,7,8-tetrahydronaphthalen-1-yl]amino}pyrimidin-4-yl)-5-(trifluoromethyl)phenylcarbamate;
trans-3-methoxy-5-({6-[4-(trifluoromethyl)phenyl]pyrimidin-4-yl}amino)-1,2,3,4-tetrahydronaphthalen-2-ol; and
trans-3-methoxy-8-({6-[4-(trifluoromethyl)phenyl]pyrimidin-4-yl}amino)-1,2,3,4-tetrahydronaphthalen-2-ol;
or any pharmaceutically-acceptable salts or hydrates thereof.

3. A method of treating acute, inflammatory and neuropathic pain, dental pain, general headache, migraine, cluster headache, mixed-vascular and non-vascular syndromes, tension headache, general inflammation, arthritis, rheumatic diseases, osteoarthritis, inflammatory bowel disorders, depression, anxiety, inflammatory eye disorders, inflammatory or unstable bladder disorders, psoriasis, skin complaints with inflammatory components, chronic inflammatory conditions, inflammatory pain and associated hyperalgesia and allodynia, neuropathic pain and associated hyperalgesia and allodynia, diabetic neuropathy pain, causalgia, sympathetically maintained pain, deafferentation syndromes, asthma, epithelial tissue damage or dysfunction, herpes simplex, disturbances of visceral motility at respiratory, genitourinary, gastrointestinal or vascular regions, wounds, burns, allergic skin reactions, pruritus, vitiligo, general gastrointestinal disorders, gastric ulceration, duodenal ulcers, diarrhea, gastric lesions induced by necrotising agents, hair growth, vasomotor or allergic rhinitis, bronchial disorders or bladder disorders, comprising the step of administering a compound according to claim 1.

4. A pharmaceutical composition comprising a compound according to claim 1 and a pharmaceutically-acceptable diluent or carrier.
